(19) United States
(12) Patent Application Publication
(10) Pub. No.: US 2025/0075594 A1
(43) Pub. Date: Mar. 6, 2025

(54) DEEP SEA SUB-SEDIMENT SLURRY INJECTION FOR CARBON REMOVAL

(71) Applicant: Sinkco Labs PBC, Lewes, DE (US)

(72) Inventors: Victor Adrian Daniel Choquet, Paris (FR); Viji Donato Thomas, Paris (FR)

(21) Appl. No.: 18/825,531

(22) Filed: Sep. 5, 2024

Related U.S. Application Data
(60) Provisional application No. 63/580,585, filed on Sep. 5, 2023.
(60) Provisional application No. 63/664,958, filed on Jun. 27, 2024.

(51) Int. Cl. E21B 41/00

(52) U.S. Cl. CPC E21B 41/0064

(57) ABSTRACT

The present invention discloses a comprehensive system for the marine sequestration of carbon by injecting a slurry of diverse residual biomasses into deep-sea sediments. Central to the innovation is a meticulously designed flexible pipe system, which efficiently transports the slurry from storage units to the seafloor, significantly reducing the energy required for the overall injection process. The system also includes an adapted bulk carrier vessel, uniquely modified for seamless biomass transport, handling, and quality maintenance. The vessel is equipped with state-of-the-art energy generation systems, emphasizing renewable sources, and incorporates environmental considerations to minimize ecological impact. The injection process is optimized through a series of specialized equipment and detailed operational parameters, ensuring precise control and adaptability to various sediment types, depths, and environmental conditions. Additionally, the invention offers several unique embodiments catering to different needs, providing a flexible, cost-efficient, and environmentally responsible solution to ocean-based carbon storage. The system leverages the vastness of the oceans and the unique properties of various biomasses, offering a cutting-edge response to the global challenge of carbon sequestration.

### CROSS-REFERENCE TO RELATED APPLICATIONS

This application claims the benefit of priority and priority to U.S. Provisional Application No. 63/580,585, filed Sep. 5, 2023, and U.S. Provisional Application No. 63/664,958, filed Jun. 27, 2024, each of which is herein incorporated by reference in its entirety.

### FIELD OF THE INVENTION

The invention encompasses systems and processes for the permanent storage of carbon or a carbon source in the ocean using a carbon rich slurry that is injected into the ocean bed substrate via an injection pump. In various embodiments, the invention encompasses methods for the design and characteristics of the injection pumps, system design, and delivery system for carbon storage. The injection process as well as the overall system comprises several ranges of dimensions relative to the composition of the carbon rich filling including terrestrial, marine, and waste liquid substances that are rich in carbon.

### BACKGROUND OF THE INVENTION

In recent years, the alarming rise of greenhouse gases, primarily carbon dioxide CO₂, in our atmosphere has been recognized as a significant global health and environmental concern. As stated by the World Health Organization (WHO), climate change, driven predominantly by human activities since the 1800 s, stands as the paramount global health threat of our era. By April 2022, human actions have propelled CO₂ concentrations to concerning levels, peaking at 420.23 ppm according to NOAA data. This surge has unleashed a cascade of environmental repercussions, from melting ice caps to elevated sea levels and more frequent and severe weather events.

Responding to this mounting crisis demands a comprehensive approach, and Carbon Dioxide Removal (CDR) technologies are increasingly seen as pivotal. The vast expanse of the ocean, with its remarkable ability to sequester carbon, is emerging as a potent solution in this endeavor. In this context, the biomass sinking and burial technique, which centers on injecting and burying organic matter, primarily terrestrial residual biomass, deep within the ocean, offers a promising avenue for long-term carbon sequestration.

The essence of this invention is the injection and deep-sea burial of organic matter. Once this biomass is injected deep into the ocean sediments, it is effectively shielded from most biological interactions, ensuring its prolonged sequestration. The deep-sea environment, characterized by extreme cold and high pressures, further inhibits bacterial degradation of the buried biomass. This combination of depth, pressure, and cold conditions effectively ensures that the carbon content of the biomass remains securely sequestered for extended durations.

A notable feature of this method is the trapping of any CO₂ that might emerge from potential biomass degradation within clathrates. This ensures that the CO₂ doesn't re-enter the water column, providing an additional safeguard against carbon release. By strategically burying the organic carbon about 10 cm beneath the seabed, the method ensures that the carbon remains isolated from the oxygen zone and benthic organisms, thus amplifying its sequestration potential and duration.

However, the path to widespread adoption of oceanic CDR methodologies, such as biomass sinking and burial, isn't devoid of challenges. Economic, legislative, and logistical barriers, as well as uncertainties surrounding the method's efficacy and potential environmental implications, have acted as deterrents. It's imperative to undertake rigorous scientific research to validate the feasibility, safety, and long-term benefits of biomass sinking and burial.

In summary, the deep, uncharted realms of our oceans hold a profound potential to aid in mitigating the climate crisis. Through the innovative approach of injecting and burying terrestrial residual biomass deep within the ocean, this invention offers a sustainable, effective, and promising solution to the pressing challenges of climate change.

The invention presented herein introduces a groundbreaking method in the realm of marine Carbon Dioxide Removal (mCDR)—the deep-sea injection and burial of terrestrial residual biomass. Capitalizing on the vastness and isolation of the deep ocean environment, this method ensures long-term carbon sequestration, significantly mitigating the risks of carbon re-emergence into the atmosphere, while at the same time offering a relatively well-balanced efficiency, and energy retirement (˜1000 MJ/tCO₂), and a very low capex cost to allow a fast scale up and operation to mitigate climate change. By injecting organic matter deep beneath the ocean floor, the innovation not only leverages the natural protective barriers of the seabed but also harnesses the extreme conditions of the deep sea to deter biomass degradation. As a pioneering approach in mCDR, this deep-sea injection technique promises unparalleled efficacy in carbon storage, paving the way for a sustainable solution to the escalating challenges of climate change.

### SUMMARY OF THE INVENTION

The invention is versatile in its ability to utilize a broad spectrum of biomasses for carbon sequestration purposes. It encompasses biomass from both terrestrial and marine origins. Significantly, residual biomass, encompassing crop production leftovers and industrial byproducts (approximately 4B MT annually), finds utility in this process. Animal manures, particularly from poultry (around 460M MT annually), cows (around 1.4B MT annually), and other livestock like horses (cumulatively about 200M MT annually) are also incorporated.

In certain embodiments, a deeper exploration of crop residual biomass reveals the method's adaptability. It efficiently employs corn stover (around 170M MT annually), wheat straw (approximately 730M MT annually), rice husks (close to 150M MT annually), sugarcane bagasse (about 270M MT annually), and even residues like soybean remnants (nearly 130M MT annually) and cotton linters (around 5M MT annually).

In certain embodiments, the system transforms manure, other slurry, into a valuable resource. The unique properties of each type of manure enhance the overall sequestration efficiency.

In certain embodiments, the commitment to environmental conservation is evident in the method's choice of non-harmful, carbon-rich chemicals. Byproducts like molasses from sugar production and wood residuals from industrial processes are also harnessed.

In certain embodiments, agricultural hubs globally, such as the US's Midwest “Corn Belt”, the “Granary of India” in the Punjab region, Mato Grosso in Brazil, the North China Plain, and the Nile Delta in Egypt, produce vast quantities of organic biomass annually, ensuring a steady supply for the inventive slurry injection technique.

In certain embodiments, the carbon content in these materials allows for their sequestration capability. It is preferable to use biomass with higher carbon content. Biochar molasses, for example, can contain up to 70% carbon by weight, equivalent to 2.57 tons of CO₂ per ton. Lignin holds 40-50% carbon, translating to 1.47-1.83 tons of CO₂ per ton. These materials, when sequestered, can offset billions of tons of CO₂ emissions annually.

In certain embodiments, tropical agricultural lands offer an advantage with consistent year-round waste production, ensuring uninterrupted operation of the methods of the invention.

Biomass Carbon Content & Properties: The biomass's carbon content influences its efficacy in this method. Biomasses like lignin are preferred due to their resistance to marine degradation. Residual organic or bio-based oils, with their high carbon content, add versatility to the process. For efficient pump operations, biomasses with high porosity and carbon content are ideal, while those absorbing large amounts of water pose challenges.

In certain embodiments, the biomass's water absorption impacts the compression process. High absorption leads to more water in the biomass, reducing compressibility and making the process less efficient.

In preferred embodiments, the biomass showcases low water absorption and high porosity, low permeability, ensuring efficient compression and a denser biomass concentration in the slurry, leading to optimal carbon sequestration.

The invention recognizes the environmental impact of using traditional diesel fuel and promotes the use of greener alternatives. This includes the utilization of biodiesel for trucks and ships. Additionally, the increase in electrified rail systems is highlighted as a sustainable means for rail transportation. Hybrid vehicles, which combine conventional fuels with electric power, are also noted as a growing preference for short-distance transport.

In certain embodiments, efficient marine transportation of biomass preferably utilizes an advanced marine transport infrastructure, which includes:
ports and docking areas capable of managing large biomass volumes;
automated systems for seamless loading and unloading of biomass onto ships; and
nearby storage facilities for short-term storage prior to shipping.

The invention suggests utilizing various vessels tailored to specific types of biomass. This approach ensures that each kind of biomass is handled and transported in the most suitable and efficient way. Different vessels can accommodate the unique characteristics and needs of each biomass type including, but not limited to, tankers, tailored for liquid biomasses like molasses, bulk carriers, best suited for vast quantities of solid biomasses such as wood residuals, or container ships, designed for pre-packaged biomasses ensuring quality retention during shipping.

In certain embodiments, the specific details of vessels include factors to consider for example,
storage capacity, ranging between 5,000 to 400,000 metric tons.
onboard equipment aiding in biomass loading and unloading.
systems to maintain biomass quality during transit, like temperature-controlled compartments for certain sensitive biomasses.

In certain embodiments, to cater to diverse biomass needs, ships have been uniquely modified. One major modification is compartmentalized storage areas, ensuring each biomass component is stored separately. The main focus is on bulk carriers due to their suitability for transporting large quantities of terrestrial crops, as they offer ample storage and robustness.

In embodiments, effective route planning includes global operations. Factors include, but are not limited to:
shortest and most fuel-efficient routes;
avoiding sensitive marine ecosystems; and
monitoring maritime conditions for safe and prompt deliveries.

In certain embodiments, the invention's environmental focus also considers fuel choice. Cleaner alternatives are preferred over traditional diesel, including:
biofuels from sustainable sources;
liquid natural gas (LNG) with lower pollution; and
electric propulsion for short-distance journeys.

In certain embodiments, the efficiency of marine transportation relies on:
vessel speed, usually between 10 to 30 knots;
shipment frequency, ranging from weekly to monthly based on demand and production;
optimized turnaround times at ports; and
compliance with global maritime regulations is also utilized.

The invention ensures:
adherence to the International Maritime Organization (IMO) guidelines;
regular vessel checks and maintenance; and
training crew members on safety protocols and handling different biomasses.

The invention's core is environmental conservation and involves:
preventing accidental biomass spillage;
established procedures for waste disposal during transit; and
systems to monitor carbon emissions for each journey, in line with the invention's aim of carbon reduction.

In certain embodiments, the journey between the port and the oceanic injection site varies, ranging from 500 meters to 3,000 kilometers. However, for operational efficiency, the ideal transport distance is between 500 meters and 60 kilometers. This strategic decision ensures cost-effective, sustainable transportation, in alignment with carbon sequestration goals.

In certain embodiments, the injection operation is centered around a sophisticated sea vessel carbon storage delivery system, designed for the marine sequestration of carbon by injecting a slurry of residual biomass into deep-sea sediments. This innovative system is meticulously crafted with a series of equipment, each serving a specific function, ensuring the seamless and efficient delivery of the biomass slurry into the oceanic depths.

### Equipment Overview

In certain embodiments, the equipment prevents accidental environmental contamination by utilizing, for example:
Airtight units securely store vast biomass quantities.
Transport Seaways: Designated marine pathways for vessel navigation.
Crane Systems: Used for loading/unloading heavy equipment.
Injection Needle: Designed for deep-sea floor penetration during slurry injection.
Flexible Pipe Systems: Transport the slurry from the storage units to the sea floor.
Winch Systems: Aid in deployment and retraction of pipes and the needle.
Pumping Systems: Ensure efficient movement of the slurry.
Slurry Mixer: Mixes the biomass into a consistent slurry.
Bulk Carrier Vessel: The primary vessel that houses all the aforementioned equipment.
Energy Considerations:
Energy Generation Onboard: Powers all operations with state-of-the-art generators.
Alternative Energy Sources: For near-shore operations, external electricity sources can be tapped.
Vessel's Energy Efficiency: Designed to be cost-efficient with an emphasis on renewable energy sources.

### Slurry Composition

Biomass-specific Slurry: Tailored according to the type of biomass.
Water Absorption Capacity: Dictates the slurry mix with saltwater.
Intrinsic Water Content: Influences slurry composition.
Description of Operation:
Corer Needle Deployment:
Integration of Flexible Tubing and Corer Needle: Ensures a leak-proof connection.
Deployment Mechanism: Ensures the needle's precise descent.
Ship Positioning and Stabilization: Ensures steady vessel positioning during injection.
Depth of Needle Deployment: Adjustable based on sediment type and sequestration depth.
Sediment Coring Weight Mechanism: Aids in needle's descent.
Activation of the Slurry Mixer and Pump: Ensures a consistent slurry mixture.
Zone of Injection:
Suitable Seabed Sediments: Calcareous, siliceous, and other sediments ideal for the operation.
Sediment Column Volume: Must be vast enough to accommodate the slurry.
Detailed Sediment Characteristics: Include turbidites, marly oozes, and carbonate content.
Grain Size and Permeability: Varies across sediment layers and influences slurry retention.
Abyssal Plain and continental shelf: Unique sediment characteristics beneficial for injection.
Arrival and Positioning of the Ship: Utilizes advanced navigation systems for precise positioning.

### Injection Process:

Flow Rate of Injection: Dictated by pump capacity and slurry consistency.
Duration of Injection: Influenced by pump characteristics, sediment nature, and vessel size.
Corer Needle Penetration and Lifting Time: Ensures efficient operation.
Scientific Basis for Carbon Sequestration:

### Geological Storage

Deep-sea Sediment Sequestration: Places carbon-rich material away from biological interactions.
Benefits of Biomass Encapsulation: Shields biomass from potential grazers and bacterial degradation.
Carbon Trapping Mechanisms: CO₂ gets trapped within clathrates.

Advantages over Other CDR Methods: Ensures prolonged sequestration periods, extending to millions of years.

### Carbon Storage Capacity

It should be noted that the methods can vary based on several factors including pump characteristics, slurry composition, injection depth, and biomass characteristics.

Adaptations of Injection Needles: Various embodiments demonstrate adaptations to the injection needle for optimal penetration, ranging in length from 10-250 meters and in diameter from 5-50 cm. These include vibro piston powered, and gravity mechanisms, catering to different depths and sediment properties.

Monitoring, Reporting, and Value Verifications (MRV): The embodiment of MRV includes the use of probes, laboratory testing, standardized tracking, and ethical standards. Visual clues through video equipment and deep-sea torches fixed on the needle corer are also incorporated to study resuspension and validate the injection process.

Injection Location and Equipment Variation: The slurry will be injected into deep-sea sediment at depths between 100-5000 meters and at least 6 km from the shore. The equipment and processes can vary in size, number, and composition as detailed herein.

Rail System and Onshore/Onboard Preparations: Embodiments include a rail system for continuous injection, onshore slurry preparation, onboard biomass generation, dry biomass injection methods, and a submersible mixer for slurry preparation.

Subsea Pump Integration and Airtight Mixer Chamber: Other embodiments involve the integration of subsea pumps, a multi-pump system for friction reduction, and an airtight mixer chamber for gravity injection.

Adapted Weight Hammer Systems and Unfoldable Grip on Needle: Additional embodiments highlight adapted weight hammer systems, unfoldable grips on the needle, corer needle adaptation with a drill, and a needle with outside spacing for liquid flow.

Flexible Pipe Systems and Bulk Biomass Transport Methods: Unique embodiments include the principal embodiment of the flexible pipe system, bulk biomass transport via conveyor piping with hydrostatic pressure adaptation, and spiral piping for biomass transportation.

Biomass Concentration Adjustment Chambers and Multi-Line Needle Packages: These embodiments focus on biomass concentration adjustment chambers and multi-line needle packages on ships.

Telescopic Reels, Rail Systems, and Increased Simultaneous Injection Capacity: Further embodiments extend to telescopic reels and rail systems, increased simultaneous injection capacity, and mobile platforms for sea operation.

Integration with Algae Bloom Recovery and Near Shore Operation: Additional embodiments cover the integration with algae bloom recovery and the potential for nearshore operation.

Additional Integration of Other CDR Approaches: This includes integration with technologies like SeaBound, UK and the introduction of a circular multi-micro injection system for simultaneous injections.

Zone of Injection and Injection Process: The zone of injection details suitable seabed sediments, sediment column volume, detailed sediment characteristics, grain size, and permeability. The injection process includes aspects such as the flow rate of injection, duration, corer needle penetration, and lifting time.

Scientific Basis for Carbon Sequestration: This covers geological storage, deep-sea sediment sequestration, benefits of biomass encapsulation, carbon trapping mechanisms, and advantages over other CDR methods.

The operation offers a cutting-edge solution to carbon sequestration, utilizing the vastness of the oceans, ensuring minimal environmental impact, and optimizing the carbon emissions-to-removal ratio. Different embodiments cater to various sediment types, depths, and environmental conditions, providing a flexible and comprehensive approach to ocean-based carbon storage.

This operation offers a cutting-edge solution to the global challenge of carbon sequestration, using the vastness of the oceans as a natural storage site, while ensuring minimal environmental impact and optimizing the carbon emissions to removal ratio.

### BRIEF DESCRIPTION OF THE FIGURES

FIG. 1 illustrates an exemplary three-step process of the organic slurry injection method. It includes examples of several organic biomass types that can serve as feedstock for the process.

FIG. 2 illustrates a exemplary bore injection hole situated in the deep-sea sediment.

FIG. 3 illustrates an aerial view of the sea vessel used for transporting the biomass. This vessel is specially equipped for the purpose of the inventive process.

FIG. 4 illustrates an aerial perspective of the ship as it carries out its injection operations. The figure elaborates on all the corer necessary equipment, showcased as an example.

FIG. 5 illustrates an additional exemplary three-step process of the organic slurry injection method using a H platform instead of a sea ship in FIG. 2, as an alternative performant method to carbon storage.

FIG. 6 illustrates the Redox cascade visually representing the chemical reactions occurring in descending depth in sediments, which relies on the breakdown of complex organic matter and availability of electron acceptors.

FIG. 7 illustrates the natural ocean carbon cycle. In the natural ocean carbon cycle, only<1% of sinking carbon is stored for >1000 years in marine sediments. In a modeled worst-case carbon storage scenario of the present invention, 99% of injected carbon is stored for >1000 years. This carbon storage scenario was determined by first-order rate kinetics modeling using empirical data and modeled rate constants and stoichiometric calculation using relevant biogeochemical processes. Modeled carbon storage scenario will need to be confirmed by pilot testing and in situ monitoring of pilot sites for 1 year to ensure aligned with maximum rates predicted by the model. However, rates of degradation should be lower than worst-case scenario model results because preliminary scientific testing demonstrates that the herein technology prevents degradation of lignocellulosic biomass by 1) bypassing the redox cascade and 2) compaction, reducing diffusive transport.

FIG. 8 illustrates the dominant biogeochemical processes in sulfate reduction zone of sediments. A) Schematic detailing the steps required for degradation of complex biomass under sulfate reducing conditions. B) Chemical reactions present in sulfate reduction zone involved in degradation of organic carbon.

### DETAILED DESCRIPTION OF THE INVENTION

The invention encompasses a system and method for the long-term storage and sequestration of carbon, derived primarily from biomass sources. This system involves a meticulous process of preparing, converting, and injecting the biomass into specific geological formations to achieve safe and effective carbon storage. The process is divided into distinct stages, each contributing to the overall efficiency and efficacy of the carbon sequestration method.

Biomass Collection, Transportation, and Preparation: The invention commences with the systematic collection of biomass from various terrestrial and marine sources. Special machinery and vehicles are employed for this purpose, ensuring that the biomass is collected in its most pristine form. Once collected, the biomass is transported to a dedicated facility where it undergoes pretreatment processes, including size uniformity adjustments through shredding and crushing to a bulk size between 10 cm to 1 cm, preferably 6 to 4 cm depending on the slurry needle inner tubing diameter.

Biomass Processing and Conversion: In certain embodiment, within the facility, the crude biomass undergoes further processing using advanced centrifugal machines that segregate the biomass components based on their densities.

### Wet Biomass Residual is:

Either dried using a sludged solar dryer, until moisture content falls below 20-10%. The biomass can then be transported with less contrast trained with bulk carrier.

In certain embodiments, wet residual biomass is directly transported via ship under appropriate conditions reducing the risk of its degradation. In this embodiment is best operated in a cold environment, as they significantly increase duration of the transport.

Integration of Molasses and Carbon-Rich Compounds: In certain embodiments, integral to the invention's process is the inclusion of molasses and other carbon-rich compounds. If operations and scale require Storage tanks designed specifically for these components can be present within the facility. In certain embodiment, Sophisticated pumping systems ensure that precise quantities of molasses and carbon-rich compounds are combined with the processed biomass. Automated machinery ensures a homogenous mixture, critical for the injection process.

Injection Preparation: The invention houses an intricate injection apparatus. This comprises high-pressure pumps, tailored to handle the unique consistency of the biomass mixture. These pumps are connected to state-of-the-art injection nozzles, ensuring a controlled and consistent injection process. The apparatus is equipped with sensors that monitor various parameters in real-time Inclinometer or Clinometer, Gyroscope or Accelerometer, Theodolite or Transit, Software and Simulation Tools, guaranteeing the precision of the injection.

Geological Assessment and Injection: Prior to the actual injection, a comprehensive geological assessment of the designated injection area is conducted. This ensures the area's suitability for long-term carbon storage. Upon confirmation, the prepared biomass mixture is injected into the geological formation using the aforementioned injection apparatus. The injection process is carefully monitored to ensure that the biomass mixture settles effectively within the geological structure.

Injection process: the injection process involves either a single injector needle or a plurality of needles operating simultaneously. These needles penetrate the seabed via various mechanisms, including vibrocoring, gravity coring, piston coring, and others detailed in subsequent sections. The penetration depth of these needles withing the sediment can range from 3 to 60 meters, or from 60 to 900 meters, tailored to specific operational requirements. The slurry is injected into the sediment using specialized injection pumps, capable of handling multiple injector needles simultaneously through manifold systems. These pumps facilitate the injection of varying slurry volumes ranging from 1 to 30,000 cubic meters per injection point, depending on the system configuration elaborated on in subsequent sections.

Post-Injection Monitoring and Maintenance: Once the injection is completed, the invention mandates periodic monitoring of the injection site. Advanced sensors and monitoring equipment are deployed to ensure that the biomass remains undisturbed and that the carbon sequestration process is ongoing. Any deviations or anomalies are immediately addressed, ensuring the integrity of the carbon storage.

Environmental Considerations and Safety Protocols: The invention places a significant emphasis on environmental safety. Throughout the process, from collection to injection, environmental safety protocols are adhered to. The method ensures minimal environmental disruption, with the corer needle injection method carefully selected to minimize resuspension (detailed below in 3.3), and the materials used, including the biomass, molasses, and carbon-rich compounds, are selected for their environmentally benign nature.

In embodiments, the biomass is transported from a ship to a desired depth using a long tubular net with a diameter ranging from 0.4 m to 10 m. The particle size of the net can range between about 0.5 mm and about 250 mm, with a preferable range of about 1 to about 3 mm for optimal performance. The net material can be made of Polyethylene (PE), Polypropylene (PP), Nylon, Stainless Steel Wire Mesh, Copper Mesh, Kevlar, PTFE (Teflon), Titanium Mesh, or Hastelloy and Inconel Meshes. In this embodiment, for the first 200 m of the water column during descent, the tube allows the action of a pump to create a flow that brings the biomass down. Beyond 200 m, the biomass will not float back up due to hydrostatic pressure and will begin to fall, as supported by various research studies on the physics of the embodiments. Gravity forces push the biomass downward at an acceleration rate ranging from about 3 m to about 40 m per minute to reach a depth of >1000 m. The net allows hydrostatic pressure to equalize in the water column. The net also helps to prevent biomass from scattering, guiding it to the desired depth where it can be fed into the Biomass Concentration Adjustment Chambers or similar systems. A funnel-like device is used to collect the biomass reaching the desired depth and feed it down to a Biomass Concentration Adjustment Chambers. In this embodiment, the net is mounted on hard tubing at one end and on the Biomass Concentration Adjustment Chambers at the other. It is deployed along with the corer needle to initiate operations.

‘In certain embodiments, the biomass is transported to the desired depth using sinking bales, either through gravity alone or assisted by additional mechanisms. These bales are collected by a submerged mixer that is attached to the corer line. Once collected, the ground biomass is then directed to the Biomass Concentration Adjustment Chambers, where it is mixed to form the slurry that will be fed into the injection pump’.

### Exemplary Embodiments of the Biomass of the Invention

Central to the invention is its adaptability to a diverse range of biomasses suitable for carbon sequestration. The methodology is designed to harness biomass derived from both terrestrial and marine sources. Beyond these primary sources, the invention focuses on the utilization of residual biomass. This includes, but is not limited to, the remnants of crop production and industrial byproducts that often go to waste (estimated at 4 billion metric tons annually [4B MT]). Furthermore, manures from various livestock sources, such as poultry (estimated at 460 million metric tons annually [460M MT]), cows (estimated at 1.4 billion metric tons annually [1.4B MT]), and horses, among others (combined estimated at 200 million metric tons annually [200M MT]), are also integrated into the process. An essential facet of the invention lies in its emphasis on environmentally benign carbon-rich compounds, ensuring that the sequestration process remains as eco-friendly as possible.

When we delve deeper into the specifics of crop residual biomass, the versatility of the invention becomes evident. For instance, it proficiently utilizes corn stover (estimated at 170 million metric tons annually [170M MT]), wheat straw (estimated at 730 million metric tons annually 2023 [730M MT]), rice husks (estimated at 150 million metric tons annually [150M MT]), sugarcane bagasse (estimated at 270 million metric tons annually [270M MT]), groundnut shell, cotton stalks (more preferred biomass due to high porosity, low water diffusion), bamboo residues, sawdust (from wood), olive pomace, coconut husk, and even residues from soybean harvests (estimated at 130 million metric tons annually [130M MT]) and the short fibers, known as cotton linters (estimated at 5 million metric tons annually [5M MT]), remaining after cotton ginning, a plurality of such, but not limited to. The inclusion of these additional biomass types further expands the applicability and efficiency of this innovative approach.

Manure, often seen as a waste product, is transformed into a resource in this invention. Each of these manures, with their distinct properties, contributes to the overall efficacy of the methodology.

The invention's commitment to environmental responsibility is further underscored by its integration of non-harmful carbon-rich chemicals.

Molasses, often a byproduct in sugar production, is another significant component in the invention's arsenal.

Industrial processes often yield wood residuals, and this invention ensures they don't go to waste.

Major agricultural hubs around the world include the Midwest in the United States, often referred to as the “Corn Belt”, which primarily produces corn and soybeans. The Punjab region between India and Pakistan is another significant hub known as the “Granary of India”, producing a large share of India's wheat and rice. Brazil's Mato Grosso state is a leading producer of soybeans. The North China Plain produces wheat, corn, and cotton, while the Nile Delta in Egypt is a major rice and cotton producer. Europe's agricultural heartland extends from France's wheat fields, through the dairy farms of the Netherlands, to the Danube Valley's diverse crops.

The carbon content in the described materials (DMS) plays a crucial role in determining their efficacy as carbon sequestration agents. For instance, biochar can contain up to 70% carbon by weight, and can be turned into a biochar molasses translating to about 2.57 tons of CO₂ equivalent per ton of biochar. Charcoal, too. Lignin, on the other hand, comprises about 40-50% carbon, equating to 1.47 to 1.83 tons of CO₂ equivalent per ton. The CO₂ equivalence for other materials varies, but collectively, when these materials are sequestered, they can potentially offset billions of tons of CO₂ emissions annually, making them invaluable in the fight against climate change.

As the agricultural seasons progress, these major hubs transition into bustling centers of activity, especially during the harvest periods. By the advent of springtime, these regions collectively generate vast quantities of organic biomass. This annual ritual of agricultural production ensures a consistent supply of materials essential for the operation of the inventive organic slurry injection method. As the harvest concludes, boats (preferred method) and transport mechanisms (rails and finally roadways, default of the latter) become pivotal, transporting these vast quantities of biomass to processing centers or directly to sequestration sites.

It's worth highlighting the unique advantage presented by tropical climates in the context of this invention. Agricultural lands situated in these tropical regions offer a distinct benefit: the production of crops is not bound by the conventional seasonal constraints experienced in temperate zones. This means that the inflow of agricultural waste in these areas is consistent throughout the year, devoid of the peaks and troughs associated with seasonality. Such a constant supply stream is invaluable, ensuring that the operations of the method can continue uninterrupted, maximizing carbon sequestration efficiency.

Biomass Carbon Content: The effectiveness of the biomass in the described method is significantly determined by its inherent carbon content. Biomasses boasting a high carbon content are naturally preferred, given their enhanced capability to sequester carbon. For instance, lignin, a complex organic polymer found in the cell walls of many plants, is especially desired due to its inherent increased resistance to breakdown by deep-sea marine microbial. This resilience ensures that once sequestered, the carbon remains trapped for extended durations, enhancing the overall efficacy of the sequestration process.

Integration of Residual Oils: The invention also identifies the potential of residual organic or bio-based oils as potent carbon sequestration agents. These oils could originate from various sources such as leftover cooking oils, waste vegetable oil, animal fats, and byproducts from the biodiesel production process. Given their high carbon content and liquidity, these oils can be seamlessly integrated into the slurry injection method, providing another layer of versatility to the process.

Biomass Properties for Pump Compatibility: For the mechanical operation of the injection system, certain biomass properties are highly preferred. Biomasses exhibiting high porosity and carbon content are desirable as they exhibit greater compressibility, making them highly compatible with pump operations. On the contrary, biomasses with high water absorption capacities pose challenges. Such materials tend to fill with water during the compression phase within the pump piping. This influx of water reduces their compressibility, necessitating greater amounts of water for slurry liquefaction. The cascading effect of this is a larger slurry volume, leading to an uptick in the costs associated with the mCDR method described. Moreover, this increased slurry volume amplifies the associated emissions, potentially compromising the environmental integrity of the process.

Impact of Water Absorption on Compression: The water absorption capacity of biomass plays a pivotal role in the sequestration process. Biomasses with high water retention tendencies absorb substantial amounts of water, which impedes their compression in the pump. Water, being incompressible, resists the forces applied by the pump, making the entire process less efficient. The molecules of such biomasses get filled with water, effectively diluting their inherent properties. As these water-saturated molecules traverse through the system, they become harder to process, given that their filled porosities counteract the compressive forces of the pump. This undesirable scenario means that for a given volume, the ratio of biomass to water skews heavily towards water. Biomasses with high water retention capacities could potentially dilute the slurry, leading to increased volumes and subsequently, higher operational costs and emissions, diluting the efficacy of the sequestration process.

Desirable Biomass Characteristics for Optimal Compression: In stark contrast to the above, the ideal biomass for the method possesses low water absorption capabilities and high porosity. Such a configuration ensures that under the pump's pressure, the biomass compresses significantly, reducing its overall volume. This compression means that for any given volume, there's a denser concentration of biomass and a reduced amount of water. The high porosity facilitates the compression, allowing the biomass to act more fluidly within the system. Thus, the desired outcome is achieved: a maximized biomass-to-water ratio, ensuring the most efficient carbon sequestration process.

Among the various types of biomass suitable for carbon sequestration, cotton stalk and Groundnut shell stands out as our preferred choices. With their unique composition, including an estimated 35-50% carbon content and favorable porosity range, cotton stalks and Groundnut shell provide a balance between environmental sustainability and sequestration efficacy. Its availability and cost-effectiveness further enhance its appeal as a primary material in our innovative approach.

Description of the Slurry Composition: The slurry composition is crucial to the success of the injection process. It consists principally of a mixture of biomass and water. The typical slurry may contain a range of 25-95% water content, could in contain 10-100% with preferable range 50-80% biomass content, and the remaining portion being biomass.

In other embodiments, bio-oil (see description above) can substitute entirely or a part of the water, with the latter serving as a lubricant for the pump pipe. The oil's high carbon content makes it an effective liquid medium that, unlike water, contains carbon. It's preferable to use cheap waste oil as much as possible, leveraging its attributes without incurring high costs. The typical slurry may contain a range of 25-95% water content, could contain 10-100% with preferable range 10-30% bio oil, and the remaining portion being biomass. Carbon-containing bio oil participates in carbon storage, and also increases pump efficiency by increasing viscosity. In the case of slurry descent through mechanical system (see the embodiment section) the water content can range from 20% to 95% water.

Solubility of Biomass: Biomass utilized in the process can be either soluble or insoluble. However, insoluble biomass is preferred as it increases its potential compression power under the effect of hydrostatic pressure in the pipe. This quality enhances the overall efficiency of the process, by once more reducing the overall slurry volume, energy need, and emission making it a desirable characteristic in the selection of biomass materials.

Information on Bioaccumulation: In all embodiments, the injected device is composed of organic and/or inorganic carbon initially originated from an organic form, or a combination thereof, resulting in no harm to the ecosystem. This approach ensures that there is no ecotoxicity for the food chain through bioaccumulation of harmful compounds. The invention expands on the process involved in the green and eco-friendly processes of crude marine biomass for the extraction of a targeted valuable compound all of while generating a residual biomass free of bio contaminants, which is described in US Publication No. 2023/0132519 A1, which is incorporated herein by reference.

Grinding of the Biomass: The grinding of the biomass is a critical consideration in the process. While it can be rough or even preferably none, depending on the size of the biomass used, the preferable option for larger biomass this to minimize or eliminate grinding to reduce a size equivalent to the compressibility of the biomass, starting form the largest functional size of the biomass bulk bits in the slurry. These are established in the laboratory for each biomass. the energy requirement of the system overall. Care is taken to ensure that this approach does not limit the flow of the pump, maintaining operational efficiency without compromising environmental sustainability.

Slurry: Adding More Biomasses, Including Oils, Carbon Dense Bio Based Slurry: In our pursuit of a carbon-dense bio-based slurry, additional biomasses and oils may be included in the composition. This approach allows for flexibility and adaptability, accommodating various types of biomass with differing properties. The inclusion of oils, particularly those with high carbon content, contributes to a slurry that is both effective in sequestration and aligned with our environmental commitments. The resulting slurry is a robust mixture, tailored to the specific needs of the method, and optimized for maximum carbon sequestration.

Customization of Slurry Composition and System Parameters: Central to the adaptability and efficiency of the method is the individualized approach to each biomass used in the carbon sequestration process. Recognizing that each biomass possesses unique characteristics such as porosity, permeability, compressibility, viscosity, density, and water absorption capacity, the method is designed to be flexible and responsive. Specific slurry compositions are formulated to match the individual properties of each biomass, ensuring an optimal balance between solid and liquid components. Flow rates are calibrated to accommodate the varying viscosities and densities, ensuring smooth transport through the system. Moreover, the selection of flexible piping (see section 3.3) is tailored to the biomass, considering factors like compressibility and water adoption. This customized approach ensures that the physical properties of the biomass are harnessed effectively, leading to an optimized carbon sequestration process. By thoughtfully aligning the slurry composition, flow rate, and piping with the inherent characteristics of the biomass, the method maximizes both environmental sustainability and operational efficiency.

TABLE 1
examples of Terrestrial biomass and properties.

| | | | Global | | | Price |
|---|---|---|---|---|---|---|
| Crop | | Estimated | Production | | Carbon | (USD/ton, |
| Residual | Estimated | Pressure for Max | (MT/year, 2021 | | Content | 2021 |
| Biomass | Porosity (%) | Compression (bar) | approx.) | | (%) | approx.) |
| Rice husk | 25-35 | 100-150 | 150 | million | 35-40 | $20-$50 |
| Wheat straw | 45-55 | 80-130 | 730 | million | 40-50 | $30-$60 |
| Corn stover | 35-50 | 90-140 | 600 | million | 35-45 | $25-$55 |
| Sugarcane | 40-60 | 75-125 | 400 | million | 30-40 | $15-$40 |
| bagasse | | | | | | |
| Groundnut | 30-45 | 110-160 | 8 | million | 40-45 | $25-$60 |
| shell | | | | | | |
| Cotton stalks | 35-50 | 90-140 | 40 | million | 35-50 | $30-$70 |
| Bamboo | 25-35 | 100-150 | Data not | | 35-45 | Price |
| residues | | | readily | | | varies widely |
| | | | available | | | |
| Sawdust | 40-65 | 70-120 | Data not | | 40-55 | $20-$50 |
| (from wood) | | | readily | | | (Varies with |
| | | | available | | | wood type) |
| Olive pomace | 45-55 | 80-130 | 2 | million | 20-30 | $40-$90 |
| Coconut husk | 50-65 | 65-115 | 25 | million | 30-40 | $15-$45 |

In certain embodiments of the herein described technology, the density of the slurry is specifically formulated to be either different or equal from that of the sediment-either substantially higher or lower. Such ratio understands ratio of the slurry density to sediment density of: 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, and from 2.0 to 3.0 in certain extreme cases. This deliberate variation in density is engineered to enhance the propagation characteristics, including shape, dimension, and behavior, of the slurry within the sediment. Such adjustments are critical for maximizing the effectiveness and success of the injection process, ensuring optimal dispersion and integration of the slurry into the sedimentary environment.

In certain embodiments, the slurry composition, predominantly consisting of agricultural residue particles and seawater, is further enhanced by incorporating binders such as paper pulp, cardboard pulp, and specifically, recycled paper pulp that is free from contaminants and bio contaminant subject to EPA biocontamination assays. This pulp acts as a binding agent, introduced in varying concentrations ranging from 1% to 10% in standard applications, escalating to 10-20%, 20-30%, and up to 30-70% in specialized scenarios. The primary function of this binder is to augment the homogeneity of the slurry mixture, particularly beneficial when hydrophilic molecules are present that might otherwise increase slurry viscosity or heighten clogging risks. The list of binders mentioned above is not restricted to those mentioned and serves to educate the reader on the technology discussed.

The addition of this binder significantly enhances lubrication throughout the piping and tubing systems, facilitating smoother transit of the slurry through the pumps and reducing both friction and the potential for clogging. The cellulose content in the pulp imparts a slimy quality to the mixture, which effectively prevents the aggregation of particles that could lead to blockages within the pipeline. This feature proves especially advantageous in pipeline sections where the tubing is narrower and the flow direction varies. Such strategic incorporation of binders not only optimizes the fluid dynamics of the slurry but also ensures a more consistent and efficient injection process, crucial for maintaining operational integrity in complex sedimentary environments.

In certain embodiments, alongside paper pulp, various other biodegradable and sustainable biomass materials can be used as binders within the slurry compositions and can be utilized depending on their availability in the geography of the injection operation. These include cellulose fiber from cotton, hemp pulp, and bamboo pulp, which are known for their robust binding properties. Additionally, agricultural residues such as wheat straw pulp, sugarcane bagasse, and rice straw pulp offer economical and eco-friendly alternatives. Algae-based pulp and corn stalk pulp also provide viscous properties that enhance slurry homogeneity, akin to paper pulp. Recycled materials like textile fibers and cardboard pulp further contribute to sustainability goals while maintaining the functional efficacy of the slurry. These materials not only improve the lubrication and flow of the slurry through injection systems but also help in reducing the risk of clogging, mirroring the benefits provided by paper pulp. Incorporating such diverse biomass materials can lead to more efficient and environmentally friendly injection processes, aligning with broader sustainability practices within the industry.

The herein invention describes a high-performance marine CDR system utilizing a wide variety of biomass, specifically designed to integrate with the carbon-negative compounds production system described by Choquet et al., in US patent application publication no. US 2023/013251 A1, incorporated herein by reference. This patent details the production of carbon-negative products through green extraction and eco-friendly processes directly integrated with a CDR method. This invention fully meets the requirements for the carbon-negative product system as described for example in section [0355], claims 1 and 3 of US patent application publication No. US 2023/013251 A1, incorporated herein by reference.

The biomass types suitable for this integrated system include, but are not limited to: Marine Biomass: Seaweed, algae, kelp, and marine phytoplankton.

Terrestrial Biomass: Agricultural residues such as corn stover, wheat straw, rice husks, sugarcane bagasse, soybean remnants, and cotton linters.

Residual Biomass: Crop production leftovers, industrial byproducts, animal manures (poultry, cows, horses), lignin, and biochar.

This enhanced integration enables the production of highly carbon-negative products, surpassing results from the original patent No. [US 2023/013251 A1]. The new CDR system described herein offers a more efficient and high-performance method for carbon sequestration, primarily due to:
a more efficient Emission to Removal Ratio (ERR) value (˜2%),
a lower cost per ton of CO₂.

Additionally, the new Carbon Removals Certification Framework (CRCF) in Europe mandates that companies can only claim their products as carbon-negative if the carbon sequestration step is directly integrated into the product manufacturing process. This legislation prevents the combination of separate processes for carbon credits and product manufacturing to claim carbon negativity. Thus, the invention described herein, when used in conjunction with the extraction and manufacturing processes from US patent application publication no. US 2023/013251 A1, incorporated herein by reference, meets this regulatory requirement, allowing companies to legitimately market their products as carbon-negative.

Similarly, legislative frameworks being established in the United States and other parts of the world highlight the necessity of integrating carbon sequestration directly with product manufacturing. By using the same biomass types and leveraging the high-performance CDR system described in this patent, the herein invention presents a competitive combined approach to manufacture highly carbon-negative compounds that comply with emerging global regulations, offering a robust and sustainable solution for reducing atmospheric carbon, while introducing, as co-benefits, a broad opening for decarbonization of industries. Please refer to the background of the invention section of US patent application publication no. US 2023/013251 A1], incorporated herein by reference for more information.

Several case examples illustrate the potential of combining this novel CDR system with the processes described in US patent application publication no. US 2023/013251 A1:

Carbon Negative Cotton Production: Cotton production typically yields a 1 to 6 ratio of cotton to residual biomass. By utilizing the residual biomass from cotton production in the novel CDR system described herein, highly carbon-negative cotton commodities and clothing can be produced. This approach leverages both the extraction processes and the advanced carbon sequestration capabilities described in the patents.

Bioethanol and Biofuel Production: Bioethanol, particularly E10 ethanol, and other biofuels like Sustainable Aviation Fuel (SAF), can be produced using agricultural residues such as sugarcane bagasse and beetroot waste. The integration of the CDR system with the biofuel production processes ensures that the residual biomass is sequestered efficiently, resulting in carbon-negative biofuels. This not only enhances the sustainability of biofuel production but also complies with emerging legislative frameworks for carbon negativity.

Integrated Production of Sustainable Aviation Fuel (SAF) with Marine Carbon Dioxide Removal (mCDR) System. In specific embodiments, the present system facilitates the production of Sustainable Aviation Fuel (SAF) as an integrated component within the marine carbon dioxide removal (mCDR) system described in the herein invention, thereby generating a high-performance, carbon-negative SAF. This integration capitalizes on, but is not limited to, the utilization of diverse biomass sources, including sugarcane bagasse, which serves as a feedstock for the mCDR system as detailed herein. Additional biomass sources applicable to this process include, without limitation, cellulosic feedstock, wood residues, beet, rice, and other biomass types commonly used in ethanol production, as referenced in US Patent Application No. 2023/013251 A1, which is fully incorporated herein by reference.

This integrated SAF production method is designed to utilize eco-friendly and sustainable processes while being directly linked to the mCDR system. The process includes, but is not restricted to, thermal conversion, biochemical conversion, and hybrid methodologies. These processes include Fischer-Tropsch (FT), Catalytic Hydrothermolysis (CH), Ethanol-to-Jet (ETJ), and Alcohol-to-Jet (ATJ) processes, but are not restricted to them, thereby enabling the production of carbon-negative SAF. The emissions reductions achievable through this process range, on a preliminary assessment basis, to approximately −1.4 kg of CO₂ per liter of SAF produced, depending on the embodiment used. The integrated system described herein produces a highly sustainable SAF, meeting or exceeding the stringent carbon negativity requirements outlined in emerging global legislative frameworks. This system, while described in specific embodiments, is not limited thereto and is adaptable to any SAF production method that can be integrated with the mCDR process.

The above exemplary products demonstrate the versatility and high performance of the combined technologies, offering a comprehensive solution for producing carbon-negative products across various industries. The ability to integrate the advanced CDR system with established manufacturing processes ensures that the inventions remain at the forefront of sustainable and eco-friendly technology.

This section explores the potential and suitability of various products that can be produced in a carbon-negative manner using terrestrial and marine biomass. By integrating the novel CDR system described in this patent, a wide range of valuable compounds can be derived from terrestrial biomass. These compounds have versatile applications across various industries such as pharmaceuticals, cosmetics, agriculture, and biofuel production, among others. The compounds listed in Table 2 below are representative examples but are not limited to those specifically mentioned.

TABLE 2
Terrestrial Biomass Compounds, Their Properties, and Applications

| Chemical Group | Compounds | Properties | Industry Application | Products |
|---|---|---|---|---|
| Polysaccharides | Cellulose | Thickeners, | Fermentation, biomaterials, | Facial cream, hair gel, biodiesel, |
| | | emulsifiers | food, agriculture, | biomethane, acetone, bioethanol, |
| | | | pharmaceuticals, cosmetics, | SAF, perfume, fragrances, ready- |
| | | | cosmeceuticals, functional | to-make beverage, hand |
| | | | food, biofuel production | sanitizer, premium vodka gin, |
| | | | | paper |
| | Hemicellulose | Anti-viral, anti- | | Food, animal feed, cosmetics, |
| | | tumor | | anti-aging cream |
| | Starch | Anti-inflammatory, | | Food, feed, pharmaceuticals, |
| | | anti-oxidative, | | cosmetics |
| | | anti-bacterial | | |
| | Pectin | Anti-bacterial | | Food, animal feed, |
| | | | | pharmaceuticals, cosmetics, |
| | | | | biomaterials |
| | Inulin | Anti-fungal | | Food, feed, pharmaceuticals, |
| | | | | cosmetics, biomaterials |
| Proteins and Amino | Glutamine | Anti-viral, anti- | Pharmaceuticals, | Pills, supplements, vegetarian |
| Acids | | inflammatory | nutraceuticals | burger, fish feed, mollusk feed |
| | Histidine | Anti-oxidative | Pharmaceuticals, | Pills, supplements |
| | | | nutraceuticals | |
| Lipids and Fatty | Linoleic Acid | Anti-cholesterol, | Pharmaceuticals, | Supplement pills, biofuel |
| Acids | | anti-inflammatory | functional food | |
| | Oleic Acid | Anti-oxidative | Pharmaceuticals, | Supplement pills, biofuel |
| | | | functional food | |
| | Palmitic Acid | Anti-fungal | Pharmaceuticals, | Supplement pills, biofuel |
| | | | functional food | |
| Minerals and | K, Ca, Mg, Na, Zn, Co, | Fertilizers | Agriculture | Fertilizers |
| Micronutrients | Cu, I, B | | | |
| | Vitamins B12, K, C, E, | Anti-oxidative | Pharmaceuticals, | Pills, supplements |
| | A, D | | functional food | |
| Polyphenols | Catechin | Anti-viral, anti- | Pharmaceuticals, | Food, cosmetics, facial cream, |
| | | tumor, anti- | nutraceuticals | anti-aging cream |
| | | inflammatory | | |
| | Flavonoids | Anti-fungal, anti- | Pharmaceuticals, | Food, cosmetics, facial cream, |
| | | bacterial | nutraceuticals | anti-aging cream |
| | Phenolic Acid | Anti-viral, anti- | Pharmaceuticals, | Food, cosmetics, facial cream, |
| | | inflammatory | nutraceuticals | anti-aging cream |
| | Tannins | Anti-oxidative | Pharmaceuticals, | Food, cosmetics, facial cream, |
| | | | nutraceuticals | anti-aging cream |
| Sterols | Ergosterol | Anti-fungal, anti- | Pharmaceuticals, | Supplement pills |
| | | bacterial | nutraceuticals | |
| | 24- | Anti-fungal | Pharmaceuticals, | Supplement pills |
| | Methylenecholesterol | | nutraceuticals | |
| Diterpenes | Dolabellones | Anti-viral, anti- | Pharmaceuticals, | Food, cosmetics, facial cream, |
| | | tumor, anti- | nutraceuticals | anti-aging cream |
| | | inflammatory | | |
| | Hydrazulenoids | Anti-oxidative | Pharmaceuticals, | Food, cosmetics, facial cream, |
| | | | nutraceuticals | anti-aging cream |
| Extended | Xenicanes | Anti-fungal, anti- | Pharmaceuticals, | Food, cosmetics, facial cream, |
| Sesquiterpenoids | | bacterial | nutraceuticals | anti-aging cream |
| Common Crops | Corn Maize | Fermentation, | Food, beverages, | Beer, ethanol, vodka, |
| | | distillation | biofuel production | bioethanol, SAF |
| | Wheat residues | Fermentation, | Food, beverages, | Beer, ethanol, vodka, SAF |
| | | distillation | biofuel production | |
| | Cotton | Textile fibers | Textiles, apparel | Clothes |
| Agricultural | Bagasse | Biofuel production | Energy | Bioethanol, biofuel, SAF |
| Residues | Olive & Olive Oil | Food production, | Food, pharmaceuticals, | Carbon-negative olives, olive |
| | | waste recovery | cosmetics | oil |
| | Corn Residues | Biofuel production | Agriculture, energy | Carbon-negative corn, maize |
| | Sugarcane Residues | Biofuel production | Agriculture, energy | Carbon-negative sugarcane, |
| | | | | bagasse, |
| | Rice Husks | Biofuel production | Agriculture, energy | Carbon-negative rice husks, |
| | | | | biofuel |

### Transport of the Biomass To Ships

The invention encompasses a structured framework for transporting biomass from its origin to the designated ports or processing units. This framework employs distinct transportation avenues, each tailored to the distinct nature of the biomass, the geographical terrains, and the prevailing infrastructure.

Roadways emerge as a foundational mode of biomass transportation, but nevertheless, for the purpose of this invention, large biomass hubs located in proximity to fluvial access (1) or a railroad are preferable to lower energy requirements and limit the emissions linked to transportation. In the absence of (1) or (2), it would be envisioned to deploy large trucks or tractor-trailers specifically for residual crop biomass, such as corn stover or wheat straw. These vehicles are designed for maximum storage capacities, ensuring efficient biomass transport. For marine-derived biomasses or those requiring controlled environments, specialized vehicles are provisioned, guaranteeing that the material retains its quality during transit.

Additionally, rail systems are integrated into the invention's blueprint. Trains, optimized for hauling extensive quantities of biomass like wood residuals or industrial byproducts, are favored especially when biomass sources are in proximity to railway lines. This strategic positioning minimizes the need for intermediary road transport.

Notably, waterways, especially river systems like the Mississippi, are recognized as the invention's most preferred transport mechanism. Utilizing barges and specific vessels, the invention harnesses these water routes to transport biomass directly from fields to ports. This method is not only cost-effective but also the least CO₂ emissive,

Powering these transportation modes are selected fuels. While diesel and gasoline are conventional, there's a pronounced tilt towards cleaner alternatives. The invention promotes the use of biofuels and underscores the potential of electric and hybrid vehicles, aligning with its overarching environmental ethos.

Geographical considerations dictate that while some biomass sources might be remotely positioned from ports, the invention ideally operates within a 200-400-kilometer radius. This strategic placement ensures transport efficiency and limits environmental impacts.

Emphasizing the Mississippi River system's role, the invention identifies it as a prime route for biomass transportation. Leveraging this vast river network ensures minimal reliance on road and rail systems, reduces infrastructure costs, and minimizes carbon leakage due to biomass decomposition.

In summation, the invention articulates a multifaceted transportation model for biomass to ships. While it encompasses diverse methods, the emphasis is clear: prioritize waterways, followed by rail systems, and then roadways, optimizing for both efficiency and sustainability, and the durable establishment of hubs in key locations.

Fuel Types for Biomass Transportation: While diesel remains a standard fuel for many transport systems, the invention recognizes the ecological implications of its use. As such, there is an emphasis on transitioning to more sustainable fuel sources. Biodiesel, derived from organic sources, is a cleaner alternative for trucks and ships. Moreover, electrified rail systems are gaining traction, further reducing the carbon footprint of rail transport. Hydrogen Fuel cells. Additionally, the potential of hybrid vehicles, especially for short-haul trucks, is being explored, marrying the benefits of both conventional fuels and electric power.

Marine Transportation Infrastructure: Central to the effective transportation of biomass to the sea is the use of a well-established marine transportation infrastructure. This encompasses: a) Ports and docking facilities equipped to handle the vast quantities of biomass. b) Mechanized systems for the loading and unloading of biomass onto vessels. c) Storage facilities adjacent to ports for temporary storage before shipping.

Vessel Types: Given the diverse nature of biomasses, the invention envisions using a range of vessels optimized for the specific type of biomass. These include: a) Tankers, specifically designed to transport liquid biomasses like molasses. b) Bulk carriers (all size), ideal for transporting large quantities of solid or granulated biomasses such as wood residuals. c) Container ships, suitable for pre-packaged biomasses, ensuring quality preservation during transit.

Vessel Specifications: The specifics of the vessels are pivotal. Factors considered include:

The storage capacity, which might range from 5,000 metric tons to 400,000 metric tons.

Onboard equipment to facilitate the loading and unloading of biomass.

Systems to maintain biomass quality during transit-including temperature-controlled holds for certain sensitive biomasses.

Ship Modifications for Optimized Biomass Transport: To cater to the specific needs of transporting diverse biomasses, certain ships under this invention have been ingeniously modified. One of the prominent modifications is the compartmentalization of storage areas. This design ensures that each distinct element of the slurry mix can be stored separately, preserving its unique properties and preventing any unwanted interactions during transit. This segmentation not only optimizes space but also facilitates streamlined loading and unloading processes. Furthermore, while various vessel types can be employed, the invention predominantly favors bulk carriers. This preference stems from the realization that the primary biomass for sequestration will be terrestrial crops, which are optimally transported in the spacious holds of bulk carriers. The structural robustness of these carriers, coupled with their significant storage capacities, makes them the preferred choice for the efficient and safe transportation of large quantities of biomass.

Route Planning: With the global nature of the operation, route planning is essential. Considerations include:

The shortest and most fuel-efficient routes to reduce the carbon footprint.

Avoiding ecologically sensitive marine areas to minimize environmental impact.

Monitoring real-time maritime conditions to ensure safe and timely delivery.

Fuel for Vessels: Recognizing the environmental ethos of the invention, the choice of fuel for marine transport is paramount. While traditional fuels like diesel are an option, there's a significant inclination towards cleaner alternatives. This includes:

Biofuels derived from renewable sources.

Liquid natural gas (LNG) which emits lesser pollutants compared to traditional marine fuels.

Electric propulsion systems, where feasible, for short-distance transits.

Operational Parameters: The efficiency of marine transportation hinges on various operational parameters. These include:

The speed of the vessel, which typically ranges from 10 to 30 knots, depending on vessel size and biomass type.

The frequency of shipments, which might range from weekly to monthly consignments based on demand and production rates.

Turnaround times at ports, optimized to ensure that vessels spend minimal idle time.

Safety and Compliance: Adhering to international maritime regulations is non-negotiable. The invention ensures:

Compliance with the International Maritime Organization (IMO) guidelines.

Regular vessel maintenance and inspections.

Training for crew members on safety protocols and the specific handling requirements of the various biomasses.

Environmental Considerations: Integral to the invention is its commitment to environmental stewardship. Aspects to note include:

Measures to prevent any accidental spillage of biomass into the sea.

Protocols in place for the disposal of any waste generated during transit.

Monitoring systems to track the carbon footprint of each voyage, ensuring alignment with the invention's goal of reducing global carbon emissions.

Distance Considerations for Sea Transport: The journey from the port to the designated injection area in the ocean can span a broad range of distances, depending on several logistical and environmental factors. The transport distances can vary from as short as 500 meters (m) to as extensive as 3,000,000 meters (3,000 kilometers). However, for optimal operational efficiency and to ensure the viability of the method, the preferred transport range lies between 500 meters and 60,000 meters (60 kilometers). This strategic distance consideration ensures that the transportation remains cost-effective, environmentally sustainable, and aligns with the overarching objectives of the carbon sequestration process.

In certain embodiments of the marine carbon dioxide removal system detailed herein, the integration of turbine technologies such as Energy Recovery Turbines (ERTs), Gas Turbines, and Combined Heat and Power (CHP) systems plays a pivotal role in enhancing energy efficiency and operational sustainability. ERTs are strategically employed to capture excess energy from the high-pressure fluids utilized in the injection processes, converting this surplus into mechanical or electrical energy that can be redirected to power injection pumps and other platform machinery (i.e. generator). This not only reduces the net energy consumption but also bolsters the system's energy autonomy.

Gas Turbines are utilized as primary power sources, providing a steady and reliable supply of energy essential for continuous injection operations. Their robust nature ensures that the energy demands of the platform are met without interruption, which is critical for maintaining consistent operation and achieving desired sequestration outcomes. Combined Heat and Power (CHP) Systems further enhance the system's efficiency by capturing and repurposing the waste heat generated by gas turbines. This heat is utilized for additional power generation or heating, maximizing the utility of the energy produced and minimizing waste.

The integration of these turbines supports a reduction in energy consumption, aligns with environmental sustainability goals by lowering greenhouse gas emissions, and reduces operational costs through enhanced fuel efficiency. Moreover, the reliability provided by these integrated systems ensures the precision and repeatability of the injection processes, which are crucial for effective carbon sequestration.

To ensure optimal performance and integration, these systems are equipped with advanced control technologies that synchronize turbine operation with the platform's needs. Regular maintenance and specialized personnel are essential to maintain system integrity and performance. By incorporating these turbine technologies, the marine carbon dioxide removal system described herein not only achieves significant improvements in energy efficiency but also sets new standards in environmental compliance and operational efficacy. These technologies, suitably adapted for offshore conditions, provide robust solutions that support the overarching goals of the invention, promoting sustainable and efficient marine environmental management.

### Injection Operation

### Exemplary Embodiments of the Equipment on the Sea Vessel for a Carbon Storage Delivery System

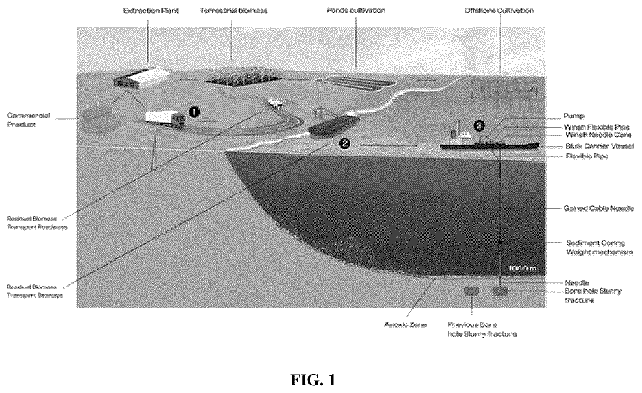

Residual Biomass Storage Units (Refer to FIG. 1): Central to the operation is the storage and management of the residual biomass onboard. These units are essentially large compartments or silos designed to hold vast quantities of biomass securely. They are constructed to be airtight, preventing moisture ingress, which might compromise the quality of the biomass. Potential alternatives include specialized containers, bulk storage bags, and modular storage tanks.

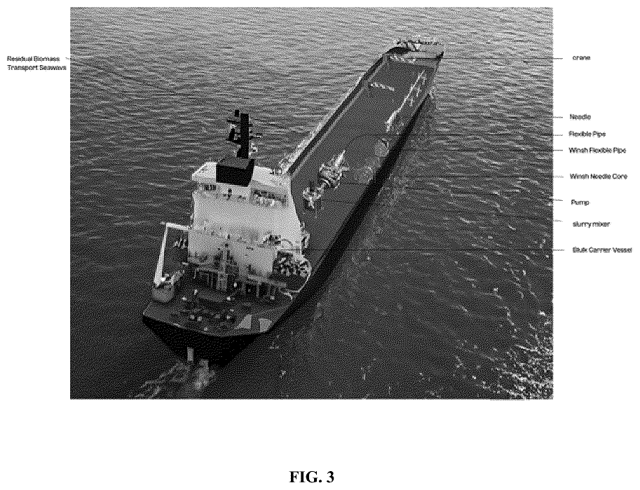

Transport Seaways (Refer to FIG. 3): These are the pathways or routes designated for the vessel's navigation from the port to the injection site. Preferably include pre-defined marine routes, GPS-guided pathways, or dynamically determined routes based on real-time data. They are mapped out considering marine safety, environmental impact, and efficiency.

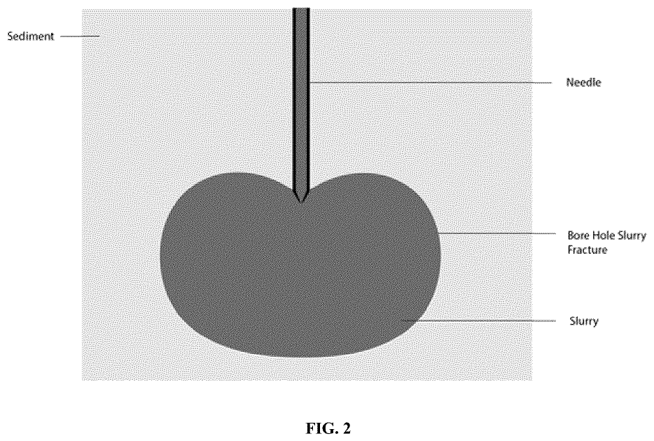

Bulk Carrier Vessel (Refer to FIG. 2): Serving as the primary vessel for the operation, the bulk carrier is equipped and modified to accommodate all the aforementioned equipment. Its design allows for the storage of vast quantities of biomass and facilitates the injection operation. The bigger the boat, the more efficient the system becomes, reaping the benefits of economies of scale. While the bulk carrier is the preferred vessel given its suitability for transporting terrestrial crops, other vessel types that could be adapted for this purpose include cargo ships, container vessels, oil tankers (especially in cases where the slurry is composed primarily of bio-oil, molasses, or other liquid organic biomass slurries as described in Chapter 1), and specialized marine operation ships.

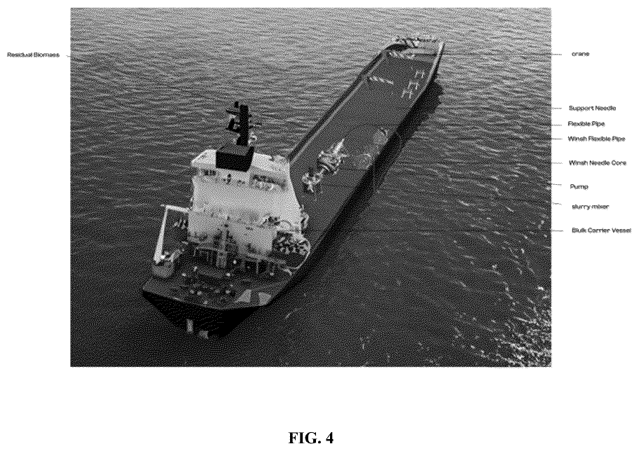

Slurry Mixer (Refer to FIG. 4): Before the injection, the biomass needs to be mixed into a consistent slurry using the mixer. This mixer is supplied with seawater, facilitated by a pump, ensuring that the slurry is mixed in the correct proportions to align with the pump capacities and optimal pipe flow rates. The mixer ensures a uniform mixture, optimizing the injection process. Potential alternatives and complements to this mixer encompass agitator systems, paddle mixers, and drum mixers.

Crane Systems (Refer to FIG. 3): Essential for the loading and unloading of equipment, especially the heavier machinery like the injection needle and pipes. The onboard crane system should have a robust lifting capacity and be designed for marine operations. Alternatives include pedestal cranes, deck cranes, and offshore marine cranes.

Injection Needle (Refer to FIG. 1,& 3): This is a specialized, elongated equipment piece designed to penetrate the deep-sea floor for the injection of the slurry. It ensures precise and deep insertion, optimizing the carbon sequestration process. Potential alternatives or complements to the primary needle include submersible probes, deep-sea augers, specialized marine drills, vibro penetration methods, piston-powered injection, and gravity-assisted devices, the injection needle can be adapted to various methodologies, such as vibro, which utilizes vibration to enhance penetration; piston powered, where a piston mechanism drives the needle into the sediment; and gravity, where the needle's weight aids in achieving the desired depth. The pointed needle would have a length ranging from 10-250 m long, preferably injecting 15-30 m3 of slurry for small injection at 30-50 m depth, 30-150 m3 of slurry for small injection at 50-150 m depth, and 150-250 m3 of slurry for injection at 150 m or deeper in the sediment. The needle's inner tube diameter can range from 5-50 cm, preferably in the range of 9-16 cm for small particle size slurry. The injection inner tube diameter would have a range from 1 cm to 20 cm, preferably in the range of 5-9 cm. These specific configurations, encompassing diverse technologies and dimensions, contribute to the adaptability and efficiency of the injection process, allowing for tailored solutions to various geological conditions and sequestration requirements.

Weight Placement on the Needle Cable: Placed on the cable of the injection needle, a weight ranging from 1 to 40 tons is utilized, with a preferable range of 4-10 tons. This weight, made of a dense material, preferably a strong alloy or metal, is designed with a hydrodynamic shape that reduces water displacement during the lifting up of the corer needle line. The hydrodynamic design serves a dual purpose. Firstly, it ensures that the needle is firmly settled in place during the injection process, anchoring it securely to facilitate precise and controlled penetration. Secondly, it counterbalances the potential effect of the needle popping out during the initial phase (TO) of injection. This thoughtful engineering enhances the reliability of the injection process, minimizing potential complications. The addition of the hydrodynamic shape emphasizes the efficiency and precision of the engineering design, considering both the functional and environmental aspects.

Winch Systems for Flexible Pipe (Refer to FIG. 3): This equipment aids in the deployment and retraction of the flexible pipes. Built for marine environments, they ensure smooth operations, especially during the injection process. Other systems that can serve the same purpose include marine-grade pulley systems, hydraulic reel systems, and drum winches.

Winch Systems for Needle Corer (Refer to FIG. 3): Specifically designed to handle the injection needle, ensuring its precise deployment and retraction. Given the needle's significance and weight, these winch systems are robust and finely calibrated. Alternatives include hoist systems, marine-capable elevators, and specialized retrieval systems.

Pumping Systems (Refer to FIG. 4): These are pivotal in ensuring the efficient movement of the slurry from the storage units, through the pipes, and into the deep-sea floor. They are built to handle the consistency of the slurry and the pressures of deep-sea operations. The pumping systems may include various types of pumps such as Centrifugal Pumps (Single-Stage, Multi-Stage, Submersible, Magnetic Drive), Positive Displacement Pumps (Reciprocating, Piston, Diaphragm, Plunger, Rotary, Gear, Screw, Lobe, Vane, Progressive

Cavity, Peristaltic), Specialized Slurry Pumps (Heavy-Duty, Hydraulic, Air-Operated Diaphragm, Pneumatic), Fractionation Pumps (Fractionating Column Feed, High-Pressure Injection, Chemical Injection), Hydraulic Pumps (Axial Piston, Radial Piston, Bent Axis), Vacuum Pumps (Rotary Vane, Liquid Ring, Diaphragm, for certain fractionation applications), Submersible Pumps (Electric, Hydraulic), Cryogenic Pumps (for low-temperature fractionation, Centrifugal, Reciprocating), Jet Pumps (Hydraulic, Steam), Mixed and Axial Flow Pumps (Mixed Flow, Axial Flow), Metering and Dosing Pumps (Electronic Dosing, Hydraulic Dosing), Turbine Pumps (Vertical Turbine, Deep Well Turbine), and others. These pumps' pressure power ranges from 200 psi to 20,000 PSI, preferably operating at ranges between 700-2500 psi for subsea pumps placed near the injection corer, and 1000 to 20,000 psi for pumps located on top of the ship. Submersible pumps, including Electric Submersible and Hydraulic Submersible Pumps, are the preferred methods for this innovation as they allow the combination with another delivery of the slurry with the appropriate local pressure, which significantly reduces the pressure requirement of the subsequent process to inject the slurry within the sediments without having to fight the hydrostatic pressure.

Subsea Pumping Systems: These are essential in orchestrating the transportation of the slurry from storage units to the deep-sea floor. Subsea pumps are engineered to manage the slurry's consistency and the intense pressures encountered in deep-sea environments. The array of subsea pumps available for this application includes Subsea Centrifugal Pumps (Single-Stage, Multi-Stage, Booster), Subsea Positive Displacement Pumps (Reciprocating, Piston, Diaphragm, Rotary, Screw, Progressive Cavity), Subsea Slurry Pumps (Heavy-Duty, Hydraulic), Subsea Injection Pumps (Chemical, Water, Mud), Subsea Hydraulic Pumps (Axial Piston, Radial Piston), Subsea Multiphase Pumps (Twin-Screw, Helico-Axial), Subsea Cryogenic Pumps (for specific applications, Centrifugal, Reciprocating), Subsea Electrical Submersible Pumps (Single-Stage, Multi-Stage), Subsea Jet Pumps (Hydraulic), Subsea Turbine Pumps (Vertical), Subsea Metering and Dosing Pumps (Electronic Dosing), Subsea Gas Lift Pumps (Gas Lift Injection), and Subsea Boosting Systems (Integrated, Stand-Alone).

These subsea pumps and systems are specifically engineered to withstand the challenging conditions found in deep-sea environments. They must deal with issues such as high pressure, varying temperatures, flow assurance, and the potential for hydrate or wax formation. Additionally, materials and sealing technologies must be carefully chosen to handle corrosive seawater and abrasive slurry materials. The specific type of pump selected for a subsea slurry injection or fractionation application would depend on various factors including the nature of the slurry, flow rate, pressure requirements, and depth of the subsea operation. This comprehensive range of subsea pumps ensures that the technology can be precisely tailored to the unique requirements of each project, optimizing efficiency, and effectiveness.

Flexible Pipe Systems (Refer to FIGS. 1 and 3, & 4): Connected to the injection needle, these pipes transport the biomass slurry from the vessel's pump units to the deep-sea floor. They are designed to be robust, enduring high pressures of the deep sea, elastic to regulate volume loss due to the hydrostatic compression of the biomass and resistive to corrosion. Alternatives encompass reinforced hoses, deep-sea conduits, and marine-grade tubing. In certain embodiment, the equipment parameters around the flexible tube could be pressure capacity displacement pump (on the ship) 10 bar, moisture content 70%, 30% insoluble wood (5 cm bulk bits max), flow rate displacement pump (on the ship)=3 m3/min, diameter tube flexible pipes 30 cm, depth outlet hole at 1000 m. Bulk density of the wood=600 kg.m3. These points are presented here so that readers have an understanding of one system, but are not limitative.

Dispension has illustrated all the machinery that is preferable for usage, as well as any other similar equipment thought to be suitable to achieve the purpose of this invention. As detailed in Section 7 (Embodiment), the list of equipment can greatly vary in numbers, size, and composition, depending on various process embodiment of the overall processes described to achieve carbon sequestration. Similarly, the order of processes is subject to variation and is reviewed in Section 7.

Energy Generation Onboard: A critical aspect of the organic slurry injection method is the energy source that powers all these operations. All the equipment, from the slurry mixers to the pump systems and winches, relies on the energy generated onboard the vessel. The vessel is equipped with state-of-the-art generators and energy management systems to ensure uninterrupted operations, even during prolonged injection processes. The onboard energy generation ensures self-sufficiency of the vessel, making it capable of performing extended operations without the need for external energy sources.

Vessel Fuel Options: The choice of fuel for the vessel is paramount, not only for its efficient operation but also in alignment with the invention's environmental ethos. As delineated in the Inventor Disclosure Form (IDF), the bulk carrier vessel can employ an array of fuel options. Traditional choices include marine diesel and heavy fuel oil (HFO). However, there's a marked tilt towards more sustainable alternatives:

Biofuels: Derived from organic materials, these present a sustainable option. Specific variations like bio-methane and bio-methanol are included.

Electro-fuels (e-fuels): These are synthesized fuels generated using renewable electricity. The list encompasses e-methane, e-methanol, and e-diesel.

Liquified Natural Gas (LNG): Favored for its cleaner burn and reduced emissions compared to conventional marine fuels.

Ammonia-based fuels: Blue ammonia, derived from natural gas, and e-ammonia, produced using renewable electricity, are both emerging contenders in marine propulsion due to their low carbon emissions.

Methanol: Both bio-methanol and e-methanol are considered, given methanol's property as a clean-burning marine fuel.

Bio-oils: These are oils derived from organic materials and can serve as a liquid fuel source for the vessel.

Hydrogen: Hydrogen can be produced from various sources, including water electrolysis using renewable energy (green hydrogen) or from natural gas (blue hydrogen). It can be stored either as compressed gas or in liquid form at low temperatures. While there are challenges related to storage, transportation, and infrastructure, ongoing research and development are addressing these hurdles, making hydrogen a promising option for decarbonizing the maritime industry.

Electricity: electricity is obtained by subsea cable in the case of large biomass hubs located nearby that generate millions of tons of biomass residuals each year.

For near-shore operations, the vessel is preferably powered using electricity, especially if sourced from renewable methods, to further minimize its carbon footprint. Despite the fact that we were not able to evaluate each of these energy sources, at this point we can still state that the preferred energy source would be one that would significantly reduce the emissions associated with this energy source production, as well as that transcribe to cost per ton of CO₂ below $70 dollars.

Alternative Energy Sources: While onboard energy generation is the primary source for most offshore operations, the invention also provides flexibility for near-shore operations. In scenarios where the vessel operates closer to the coast, it is preferably connected to external electricity sources via specialized cables. This not only ensures a constant power supply but can also tap into greener energy grids, further enhancing the eco-friendly ethos of the method. This approach can be particularly advantageous in regions with well-established renewable energy infrastructures, tropical weather generating a constant flow of biomass.

Vessel's Energy Efficiency and Carbon Performance: At the heart of this invention lies a system meticulously engineered for peak performance in terms of cost-efficiency and carbon storage capabilities. It's designed with a keen emphasis on minimizing capital expenditures, ensuring that the operation remains cost-competitive. However, it's worth noting that the system, while efficient, has a pronounced reliance on the vessel's energy sources, particularly to power its various onboard equipment. The pump, a critical component, stands out for its substantial energy demands.

Given the primary objective of this method-carbon sequestration—the choice of energy source becomes even more pivotal. It's not merely about powering the vessel and its machinery but doing so in a manner that optimizes the carbon emissions to removal ratio of the Marine Carbon Dioxide Removal (MCDR) method described herein. Renewable energy sources, such as wind, solar, or hydropower, are especially favored. When the system taps into these cleaner energy options, it elevates its environmental performance, ensuring that the carbon sequestration process is not just efficient but also has a minimal carbon footprint. In essence, while the system's energy demands are significant, by choosing renewable and sustainable energy sources, the invention ensures its environmental integrity is uncompromised.

In alternative embodiments, the aggregate pumping power of the injection pumps spans a range from 200 psi to 20,000 psi. Ideally, these pumps operate within the range of 300-2500 psi, particularly when engaged in a system of simultaneous clustered injection. It's important to note that the pressure requirements for a single injection typically vary between 2 to 140 bars, with the average falling within the range of 2 to 15 bars for mud sediment, and 5 to 40 bars in sand, and 2 to 40 bars in silt. However, these ranges are not exhaustive and may extend beyond these limits.

The injector needles and the systems previously described may be referred to by various names including Sinkcore Needle Injectors, Sinkcores Injectors, Injector Lances, Javelin Injection Needles, Multiple Injection Needles, Corer Needle Injectors, Injection Rods, and Injection Javelins, Injection nozzle, nozzle lance. However, these terminologies are not restricted to these terms and any equipment that matches the description and functionality for the purpose of this herein CDR invention are part of the herein invention. Each term encompasses the corer technological attributes and operational capacities essential to the innovative deployment of the injection mechanisms within sedimentary environments.

In the present invention, established technologies from the coring and grouting industries are synergistically integrated to forge a novel injection system specifically optimized for carbon sequestration. This system leverages the robust mechanics of gravity or other coring methods for precise sediment penetration combined with the advanced delivery mechanisms of grouting injection needles. This cross-industry innovation enhances the functional capabilities of each component while addressing unique challenges in subsurface carbon storage. Key improvements include optimizing slurry delivery through clustered, simultaneous injection that allows for rapid deployment and operation turnaround, as well as minimizing ecological disturbance. As this field evolves, further innovations specific to the nuanced demands of carbon sequestration are anticipated, marking the beginning of a specialized suite of technologies dedicated to environmental preservation. While the design of the injection needle described in the herein invention is novel, it is composed of several parts that have been developed in other industries such as oil and gas, grouting, scientific coring, civil engineering, and more. The design of injector needles employed in the herein described technology exhibits various configurations and essential features to enhance seabed penetration and slurry dispersal. Various needle configurations, such as those employed in standard grouting and coring operations, have been evaluated for compatibility with the novel injection system. The selected designs have been adapted in to meet the unique pressure, size, slurry biomass loading and compositional requirements, of marine sediment injection, ensuring minimal dispersion and maximal retention at targeted depths.

In certain embodiments of this invention, adaptations of previously designed injection lances, commonly utilized in the oil, gas, and grouting industries, are applicable to enhance the functionality of the novel carbon dioxide removal (CDR) method described herein. These adaptations would necessitate modifications in dimensions, materials, and specific structural components to align with the operational demands of subsea sediment injection. Relevant lance designs that can be adapted for this purpose include the Jet Grouting Lance, Compaction Grouting Lance, Fracture Grouting Lance, Permeation Grouting Lance, Cone Injection Lance, High-Pressure Lance, Adjustable Flow Lance, and Spiral Nozzle Lance but are not restricted to. Each of these designs, tailored for marine sediment conditions, offers distinct advantages in ensuring the effective distribution and long-term stability of injected biomass, thus contributing to the efficiency and reliability of the CDR process under the marine sedimentary environment.

In certain embodiments of the present invention, adaptations of specialized injection lances from the oil and gas industry are contemplated for enhanced functionality in subsea sediment injection for carbon sequestration. These adaptations would require modifications concerning size, material, and operational parameters to align with the unique challenges presented by marine sediment environments. Pertinent designs from the oil and gas sector that hold promise for adaptation include High-Pressure Fracturing Lances, Cementing Lances, and Directional Drilling Lances. Each of these lances offers distinct advantages such as high-pressure fluid handling, precise slurry deposition, and robust structural integrity, which are suitable for the successful implementation of the described CDR method. With tailored modifications, these technologies significantly contribute to the efficiency and effectiveness of the injection process, ensuring the long-term stability and reliability of carbon storage within subsea sediments.

Commonly, the needles feature a spiked head, optimizing initial penetration into the sediment. Alternatively, some needles incorporate a screw thread design, facilitating deeper penetration into seabed layers, as delineated in prior descriptions [V1 189].

Injector needles are typically equipped with lateral holes allowing horizontal directional injection, strategically positioned to facilitate slurry ejection into the surrounding sediment. This configuration is particularly advantageous over designs with bottom-open holes lance, where resistance from the sediment at peak injection times (TO) could otherwise lead to ejection of the injection needle corer out of the sediment. As previously described in subsequent paragraph, the addition of a weight atop the injector needle can counteract this resistance, stabilizing the needle during slurry discharge. sepistpiIn various embodiments of the present invention, the configuration of horizontal nozzles apertures also referred as ports, orifices, channels, outlets injectors on the injection shaft can be extensively customized to suit specific operational requirements. The number of nozzles apertures can range from 1 to 4 in initial designs, increasing to 4 to 6, and further to 6 to 20, and 20 to 500 in more advanced configurations. This variability is dependent on several factors including the diameter of the shaft, the consistency and size of the slurry, and the specific properties of the sediment into which injection is being carried out. In certain designs, the shaft may feature only two horizontal nozzles apertures strategically positioned to generate a directional horizontal flow that optimizes slurry dispersion. Alternatively, other designs may incorporate a single nozzle aperture to create a focused, mono-directional injection fracture, effectively channeling the slurry into targeted sediment layers with precision. These variations allow for tailored injection dynamics that can be adjusted to maximize efficiency and effectiveness in carbon sequestration operations.

In certain embodiments, the number of nozzles apertures within the injection lance are randomly placed and greatly vary in numbers and size and shape. In other embodiments, the number of nozzles apertures placed vertically on the shaft greatly varies between 1 to 1000, with different spacing from 1 cm to 50 m.

In terms of dispersion, the injector needles can feature multiple nozzles apertures arranged at varying heights along the shaft, allowing for simultaneous multi-level slurry fracture. Initial designs of the needles included four holes positioned near the base, adjacent to the spiked head. Subsequent advancements have introduced varied geometries for these nozzles apertures, including fissures, squares, or other shapes conducive to different flow velocity, direction, size and quality with purpose to reinforce the injection efficiency, reducing friction, avoid clogging, efficient slurry dispersion and controlled slurry propagation, therefore minimizing the travel of the slurry to large area, but instead increase its containment near or in specific area adjacent to the injection point.

In certain embodiments, the diameter of the nozzles apertures holes is meticulously tailored to correspond to the average particle size of the biomass slurry. This relationship is expressed through ratios ranging from 1 to 0.5:1 and from 0.5 to 0.1:1, representing the ratio between the length of the slurry particles and the length diameter of the nozzle apertures. In further embodiment, this ratio goes further from 0.1 to 0.001:1 and lower. This precise calibration ensures that the nozzles apertures dimensions are suitably matched to the particle size, optimizing the flow and distribution of the slurry during the injection process. In certain case, this ratio also understands nozzle apertures that are smaller than the average size of the particles presents in the slurry ranging from a ratio of 1 to 2:1 (length of the slurry particles: length diameter of the nozzles apertures).

The structural orientation of the needle also plays a critical role in the operational dynamics. For instance, in certain embodiments, a small metallic piece positioned above the spiked head within the needle's shaft facilitates direct slurry flow into the sediment. This design ensures the slurry maintains its course during injection, preventing any deviation that could lead to clogging or inefficient dispersion.

Furthermore, the transition of slurry from a vertical to a horizontal trajectory is seamlessly managed by a component that adjusts the directional angle of injection. This adjustment is crucial to prevent clogging at the transition point, a common issue when shifting injection directions. To combat sediment intrusion during penetration, which can lead to clogging, the needles may incorporate retractable traps or valves. These components dynamically open or close during different phases of the injection process, ensuring clear passage for the slurry while preventing sediment backflow into the needle. Overall, these innovations in injector needle design not only enhance the effectiveness of sediment penetration and slurry dispersion but also significantly improve the operational reliability and efficiency of the sub-sediment carbon storage system.

In certain embodiments, the injection lance is equipped with specialized caps on its apertures, designed to accumulate pressure to a predefined threshold before allowing the slurry to forcefully burst out. This mechanism, inspired by techniques used in the oil and gas industry, is adapted to meet the specific characteristics of seabed and consolidated sediment environments among others. The controlled sudden release of pressure enhances the directional precision and behavioral consistency of the slurry during the injection process, thereby optimizing the distribution and efficacy of the substrate within the sediment.

The corer needle injector, central to the marine CDR system outlined in this invention, represents a pivotal innovation in biomass injection technology. This injector harnesses unique attributes that distinguish it from conventional models, driving enhanced performance and efficiency in carbon removal. Its design innovation facilitates two key functions: swift deployment into sedimentary receivers and simultaneous, pressure-effective injection processes. These advancements are at the heart of the technological breakthroughs presented herein. What sets this technology apart is its seamless integration of the injection lance with an advanced penetration system, revolutionizing biomass injection efficiency beyond conventional industry standards. This integration not only elevates the functionality of standard oil and gas injection processes but also represents a significant leap in adapting and refining established industrial techniques for optimized carbon storage in sub-sediment environments.

In addition to the current innovations presented herein, the novel design of the injection needle anticipates the development of specific pieces of equipment tailored to enhance the efficiency and effectiveness of the carbon sequestration process. These future innovations, which will be detailed in subsequent patent filings, are expected to further refine the integration of coring and injection technologies, optimizing them for sub sediment applications. This planned expansion of the technology base underscores the invention's scalable and adaptable design, poised to evolve with the advancing needs of carbon sequestration efforts globally. The foundation laid by the current invention provides a versatile platform from which numerous specialized tools and methods can be developed, each potentially meriting individual patent protection as part of an expanding patent family. Future iterations of the injection needle may incorporate advanced materials and geometric modifications to further enhance performance, drawing upon evolving technologies in both the grouting and marine engineering sectors.

In certain embodiments, the diameter of the corer needle employed can further range from 20 cm to 30 cm, extending to 30 cm to 50 cm, and in some extreme cases, from 70 cm to 100 cm. This variance in diameter specifically accommodates the injection requirements for mud sediment but does not restrict it. Mud and other sediment presenting low permeability and porosity and transmissivity, consequently minimizing the diffusion, displacement, and diffusion of the water contained in the slurry, as well as the diffusion of borehole pressure created in these types of sediment. Utilizing larger diameter tubing facilitates injection at substantially reduced flow velocities. Such a low flow velocity is critical as it diminishes the likelihood of biomass migration toward the surface of the sediments and back into the overlying water column, while still maintaining an effective flow rate for overall performance of the CDR system described here.

In this embodiment, the pressure requirements escalate with increasing needle diameter due to the greater cross-sectional area needing displacement, thus encountering heightened sediment resistance. However, this configuration is particularly advantageous for mud medium where pressure requirements are typically minimal, often ranging between 2 bars to 6 bars for standard tubing size (1 to 4 inch). This setup proves even more beneficial in shallow mud settings at lower depths, where sediment is less dense and compacted, and the water column height is typically under 60 meters-though the system remains versatile across all described ranges of water column heights and sediment types.

Employing a larger pipe diameter not only facilitates a lower slurry flow velocity but also allows for the escape of water, potentially leading to various and numerous ramifications or veins each with various patterns and shape. These patterns can range from purely horizontal to vertical or any angle in between, from 1 degree to 90 degrees. The slow progression of these ramifications ensures that biomass is effectively encapsulated within the sediment matrix, while allowing the water to travel though the sediment. In this embodiment, the slower flow velocity of the slurry mixture through the constructed ramifications is directly influenced by the larger diameter of the injection lance. With increased lance diameter, the particles within the slurry mixture exhibit a propensity for clogging and tend to travel at significantly slower speeds compared to the water component. This distinct flow velocity differential facilitates a natural separation of the solid content from the liquid within the slurry, effectively constraining the solid particles closer to the injection point. Consequently, this method optimizes the spacing between each injection point, whether vertically or horizontally oriented, allowing for more effective simultaneous clustered injections. This optimization ensures that the integrity, containment and effectiveness of the slurry injection are maintained, enhancing the overall efficiency of the carbon sequestration process. This method is particularly effective with larger particle sizes, ranging from 1 cm to 8 cm in average but does not restrict to that size.

In certain embodiments, the size and configuration of the nozzles apertures on the injection lance are paramount. While the overall diameter of the injection lance lays in larger range of diameter 3.5 inch and above (eq to 9 cm ø and above), the dimensions of the nozzles apertures themselves play a crucial role in determining the flow velocity of the slurry. For instance, an injection lance with a diameter of 3.5 inches may feature two horizontally aligned tubes of the same diameter. In this setup, the flow velocity of the slurry within the lance is effectively halved, as the cross-sectional contact area with the sediment doubles relative to the internal cross-sectional area of the lance. Expanding this configuration to include four horizontal nozzles apertures, each 3 to 3.5 inches in diameter, further reduces the flow velocity by a factor of four. Concurrently, the pressure within the system increases due to the greater surface area of contact with the sediment that is displaced. This detailed attention to the nozzle's apertures configuration enhances the control over the injection process, ensuring optimal distribution and efficacy of the slurry deployment within the sediment matrix.

In certain embodiments, achieving a lower flow velocity and subsequent effective slurry deployment does not necessarily require large-diameter tubing. Medium range corer needle tubing, for example, measuring 3.5 inches in diameter, can incorporate multiple 3-inch injection aperture. These nozzle apertures, numbering up to 4 on the horizonal plane of the injection lance, are strategically positioned by set relatively close to one another on the vertical height of the corer needle shaft-ranging from 10 to 40 cm, 40 to 1 m, 1 m to 3 m apart or more —and can be directed in several orientations of the horizonal plane injection. In this configuration, several sets of four nozzle aperture present on the horizontal plane at different vertical heights totals a number of 4 to 40 or even more in certain embodiments. This configuration reduces the overall flow velocity of the injection commensurate with the collective cross-sectional surface area of the nozzle aperture. Consequently, the pressure requirements within the system increase to compensate for the enhanced surface area interfacing with the sediment, optimizing the injection process for efficiency and effectiveness in sediment penetration and slurry distribution.

In certain embodiments, clogging events can occur while the injection lance penetrates the sediment, particularly when different sediments accumulate and compact within the lance, potentially leading to premature clogging. To mitigate such clogging, several strategies are employed. Initially, the injection process may commence with a water, seawater, purge for the first seconds to minutes. This is facilitated by a tank positioned on the injection vessel, with manifolds arranged to introduce water into the sediment to initiate fracturing before the slurry is injected. This technique where water is used to start the fracture process is utilized in the oil and gas industry on land, particularly during the injection of biosolids and drill cuttings into Type V wells and is suitable in the current system of the herein invention but not always required, due to the minimal pressure requirement of the injection in sediment.

Additionally, preferred embodiments within this invention incorporate mechanical systems equipped with trapped metal devices designed to open and close the nozzle apertures. These devices are activated to open the nozzles at the onset of injection, ensuring clear passage for the slurry, and are closed during the retraction of the lance to prevent sediment ingress, thereby optimizing the integrity and functionality of the injection process.

In the realm of sub-sediment slurry injection for carbon sequestration, established methods from the grouting and oil and gas industries offer viable solutions to the challenge of clogging during lance penetration. These industries utilize various anti-clogging technologies such as mechanical shutters, backflow preventers, integrated filters, vibratory systems, chemical agents, pulsed injection techniques, hydraulic clearing, and rotational lances to maintain clear injection pathways. Such systems are applicable to the current invention, albeit requiring specific adaptations to accommodate the unique characteristics of marine sediments and the biomass slurry composition. Modifications include adjustments in mechanical design, material selection, and operational parameters to optimize performance under marine sediment conditions. These adaptations are described in subsequent patents, enhancing the core technology detailed in this invention while ensuring robust, efficient, and clog-free slurry injection into sub-sediment layers.

In certain embodiments, the methods outlined previously for preventing clogging may not be required as the penetration velocity of the injection lance through the sediments prevents sediment ingress into the lance. This phenomenon, however, is not universally applicable across all sediment types and is influenced by a variety of factors. These include the density and type of sediment, the penetration velocity of the lance, the mechanisms employed for penetration, the diameter of the injection tubing, and the specific properties of the sediments encountered at each injection layer. This variance underscores tailored approaches based on the geological characteristics and operational conditions specific to each injection site.

In certain embodiments, a specialized design includes a slight recess within the corer needle where the nozzle apertures are positioned. This design features larger corer needle walls to accommodate an internal viewing window, aiming to further limit sediment ingress into the injection lance, thereby reducing the potential for clogging. In other embodiments, the injection needle is kept filled with water during penetration, enhancing its ability to prevent sediment from entering and clogging the lance. Additionally, some embodiments incorporate an advanced pumping system, either integrated within the primary injection unit or as part of a supplementary pumping unit linked to the injection system. This system is capable of delivering high-pressure pulse water injections that are activated once the lance is withdrawn from the sediment. This functionality is designed to expel any accumulated sediments within the lance before it is re-inserted into a new injection area, ensuring smooth operation and preventing clogging during subsequent injections.

In reference to the depth of needle deployments within the sediments as outlined in paragraph [147, V1], the variable depth of injection can ranges based on the employed methods of needle insertion to the seabed. In certain embodiments, needle injections achieve depths varying from 1 to 3 meters, 3 to 20 meters, or 20 to 60 meters. Furthermore, other embodiments facilitate deeper penetrations ranging from 60 to 900 meters, depending on the penetration mechanisms employed, which are detailed in subsequent sections of this disclosure.

In certain embodiments, the additional pump models include shear centrifugal pump, colloid mills, high-shear mixers, emulsifying pumps, disperser pumps, homogenizer pumps, in-line mixers, ultrasonic mixers, static mixers, rotor-stator pumps, high-pressure homogenizers, turbulent flow mixers, agitator pumps, hydraulic shear pumps, progressive cavity mixers, and viscosity enhancers.

In specific embodiments, a tailored Johnson sieve, here also referred to as the Johnson Sieve Corer, Johnson Gravity Corer, Johnson Gravity Sinkcore, Johnson Injection Nozzle, Johnson Injection Rods, Johnson Injection Lance, Core Injection Lance, and any other terms referring to the Injection Lance that integrates a section of the Johnson sieve, is utilized to alleviate the pressure accumulation often encountered during injection processes, particularly in sediment types such as mud, which exhibit low permeability, porosity, and water transmissivity. This design proves essential in shallow injection scenarios where the water-saturated mud may cause the slurry to ascend through paths of least resistance due to the vertical fracture stresses being surpassed by horizontal stresses. To address potential inefficiencies that arise when slurry escapes to the surface and reenters the water column, the corer or modified Johnson sieve equipment described here directs the flow. Instead of allowing the excess water present in the slurry to displace mud and surface passively, the sieve provides an alternate path for the liquids, effectively handling the redirection of flow due to its construction.

In certain embodiments, the Sieve Johnson Sieve-Corer employs a penetration mechanism akin to that of the injector corer needle, which may include modified gravity coring, vibro-coring, piston coring, or other established methods. The Johnson sieve-corer is strategically positioned at pre-determined locations known to intersect with slurry propagation paths and fracture lines. The distances from the main injector corer to the Johnson-Corer can range broadly, including intervals such as 10 cm to 1 m, 5 m to 15 m, and extending up to 40 m to 50 m.

Standard configurations position the Johnson Sieve on the horizontal plane corresponding to the horizontal fracture, although adjustments are made based on the directional angle of injection. Exemplary angles may vary from a direct horizontal path (90 degrees) to downward angles between 90 to 125, and 125 to 140 degrees, and upward injections from 90 to as low as 20 degrees, depending on the specific requirements of the nozzle injector design, type of sediments and in certain case for the configuration of the clustered injection system, if any.

In certain embodiments, the Johnson Sieve Corer features a top section akin to the injection corer needle but diverges in design by integrating an open tube structure where water from the injected slurry can freely escape. The outlet section of the top is optimally positioned at distances ranging from 1 to 10 meters, ideally between 3 and 6 meters, to prevent vigorous water movement near the seabed that could create plumes and potentially disturb benthic environments with suspended organic particles. The sieve corer extends downward, featuring a variable-length sieve portion that could span 1 to 20 m, or more in certain case, designed to facilitate the outflow of water while adapting to the flow velocity, volume, and diameter of the injection corer needle. The design includes a spiked head similar to the injection needle for effective sediment penetration, though variations in shape are possible to accommodate different sediment types.

The sieve section of the Sieve Corer is precision-engineered to allow water passage while restricting most sediment particles, offering particle passage tolerances from 0 to 5%, 5% to 20%, 20% to 50%, 50% to 70% without risk of clogging. Advanced designs incorporate anti-clogging features such as retractable metal protectors that shield the sieve during penetration and reveal it during injection, or an internal corer that can slide or rotate to open and close the sieve, thereby preventing sediment blockage. This selective permeability ensures that all or the great majority of biomass particles are retained at the targeted depth within anoxic sediment layers, enhancing the injection process's precision and efficacy.

In configurations requiring handling of high-volume slurry injections at rapid flow rates, multiple Johnson sieve corers are deployed within a single injector setup to effectively manage water and pressure distribution across the permeable sediment. Each corer is aligned with individual fracture paths created by the injection lance, ensuring that each fracture is equipped with a corresponding Sieve Corer to manage the outflow dynamics effectively.

In certain embodiments, particularly in the initial design of this invention, the injection lance is equipped with four nozzle apertures situated above its spiked head. These apertures are arranged horizontally and are replicated at various heights along the lance shaft. Correspondingly, four corer sieve units, embedded within the lance, align with the horizontal paths of the slurry fractures generated by these nozzles. Each sieve unit is positioned at heights matching those of the nozzle sets on the injection lance, facilitating a stratified, multi-level injection process. This setup allows for multiple, clustered injections to be executed vertically, with each stage enabling four distinct fracture injections. This method employs the corer sieve design to successfully manage and direct the injection process, ensuring efficient and targeted slurry delivery into the sediment.

These sieve cores may be packed with highly porous materials like gravel or sand to trap incoming sediment, or alternative materials like charcoal may be used to facilitate water transfer back to the water column, optimizing the injection process. This system not only ensures the containment of the slurry at the intended sediment depth but also reduces the pressure requirements of the injection by providing an easier path for pressure dissipation, which would otherwise require displacing sediment to accommodate the extra water. This innovative sieve design supports a highly efficient injection strategy, accommodating complex sediment characteristics and allowing for effective carbon storage at varied depths and under different sedimentary conditions.

In certain embodiments, this design is specifically tailored for a system of inline injectors positioned on an injector platform (H-platform or sea bulk carrier vessel), typically arranged sequentially. The initial corer injector within this arrangement dispenses slurry horizontally in a unidirectional pattern, flagging the sediment for subsequent interaction. The resulting fracture and its radius are strategically designed to intersect with the trajectory of the following dual injector/sieve needle. This second injector, equipped with a sieve section opposite the corer and oriented towards the first injector, captures and dissipates water and pressure while managing the outflow from the initial fracture. This dual injector then initiates a slurry fracture directed towards a third dual injector, and so forth, in a linear configuration. In setups involving six injectors in line, the first and last injectors function as single, unidirectional injectors and a sieve, respectively, while the four central units operate as dual sieve/injector systems.

This design enables multi-level injection by employing a stratified mechanism where each injection occurs at different heights and depths within the sediment layer. The dual sieve/injector units, being slightly larger in diameter and more complex in their internal structure than standard injector nozzle or sieve corer previously described in the above paragraph, are inherently more costly but reduce the necessity for addition core penetration (sieve corer injector) which is the case of the design described in the previous exemplary embodiment into the sediment bed while allowing integration of the sieve corer design in the standard initial design without the necessity of additional core penetration the seabed, therefore reducing the risk of ecological disturbance of the benthic biota. As previously described for standard injector lances, the spacing between injectors is crucial to make sure slurry propagation does not intertwine together, and potentially limit successful containment of the slurry. The standard injector lances' distance, initially ranging from 1 to 60 meters with a preferred range of 10 to 30 meters, presents much reduced spacing capacity distances in the herein embodiment design, which reduces the spacing to a preferable range of 5 to 10 meters in preferred cases. This adjustment enhances the spacing between injectors, thus augmenting the number capacity on injector inline setup on the injection vessel, therefore increasing the overall performance of the invention.

This system proves especially advantageous in shallower waters and challenging sediment types like low porosity, low permeability muds. It supports improved multiple injections in line with no additional core penetration requirements, is suitable to carry vertical simultaneous injection and adaptable through gravity-coring or piston-coring methods. Additionally, it optimizes pressure requirements by reducing the need for sediment displacement, which correlates directly with the energy that would have been required to displace the volume of water here dissipated thought the sieve, within the mud. This setup not only prevents leak back, which could undermine the effectiveness of the invention, but also enhances the certainty of slurry containment and the permanence of carbon storage, ensuring environmental compliance and effectiveness over millennia.

### Slurry Composition

Biomass-specific Slurry: The method is tailored to cater to diverse biomass types, and consequently, the slurry composition is not a one-size-fits-all. For each biomass type, a specific slurry of predetermined composition is synthesized onboard the vessel. The composition of this slurry is intricately linked to the innate properties of the biomass in question, as detailed in the first chapter. Its interaction with water and other additives, such as liquid molasses or organic oils, is fundamental in determining the resultant slurry composition.

Water Absorption Capacity: As underscored earlier, the water absorption potential of a biomass is instrumental in dictating its ratio in the slurry mix with saltwater. A biomass with high water retention would necessitate adjustments in its slurry composition, ensuring the optimal performance of the sequestration process.

Intrinsic Water Content and Physical Properties of Sediment and Biomass: The inherent water content within the biomass is another critical factor influencing the slurry composition. Beyond just water content, other intrinsic properties like the biomass's elasticity, Young's modulus, and its behavior under compression play a vital role. The viscosity of the resultant mixture, especially when combined with water, is a property that cannot be overlooked, given its influence on the pumping and injection processes.

Sediment Characteristics for Preferred Injection Site (30 m deep within the sediment): Density, or weight per unit volume of the sediment, typically ranges from 1200-1700 kg/m3; for the purpose of this description, we could assume 1400 kg/m3. The porosity, the fraction of the sediment's volume that is void space, is ranging from 10 to 30%, 30% to 50% and 50 to 80%. The permeability, a measure of the sediment's ability to transmit fluids, is assumed to be 10-5, 10-5 cm/s. The mechanical properties are defined by a Young's modulus of 3 MPa (at 8 m depth), shear modulus of 100 MPa, and an average shear strength of 2 kN/m² or 2 kPa or 2000 Pa or 0.002 MPa, with literature reporting up to 10 kPa. The friction ranges between 0.46 and 0.27, while the Poisson's ratio is 0.470. Yield strength and tensile strength are not applicable in this context. These characteristics provide a comprehensive understanding of the preferred sediment for the injection process, aiding in the optimization of the methodology. It ensures compatibility with the desired sediment depth of 30 m, aligning with the sequestration objectives. It should be noted that the described conditions and characteristics are not limited to these specific values and ranges, and variations may be considered based on specific requirements and environmental considerations.

General Characteristics of the Slurry: While the slurry composition is biomass-specific, there are overarching characteristics that it invariably adheres to:

Fluidic Behavior: Paramount among these is the slurry's behavior as a fluid. Ensuring the slurry acts as a fluid is critical for the optimal operation of the pumping systems. This fluidic behavior is largely contingent on maintaining the right water-to-biomass ratio. It is imperative that the slurry behaves as a liquid, particularly under pressures ranging from 5-500 bar. For operations at varying depths, the preferable pressures are as follows: 2 to 50 bar for 1000 m depth, 50-300 bar for 2000 m depth, 100-400 bar for 3000 m depth, and 200-500 bar for 4000 m depth. These are indicative ranges and can be adjusted as required by specific operational needs. Overall, there's a clear correlation between the quantity of slurry that could be injected and the depth, guiding the method to be adaptable to diverse marine environments.

The location of the slurry injection will be within the deep-sea sediment, at a depth ranging from 100 meters to 5,000 meters, and at least 6 km from the shore. The selection of this specific location is determined by both the depth and the type of sediment, which must allow for the penetration of the needle. Additionally, the sediment properties must be suitable to facilitate the injection of the slurry at the required values, as described in previous paragraphs.

In the various embodiments of this invention, the operational pressures required by injection pumps are meticulously calibrated to accommodate diverse sedimentary and marine environments, influenced by a range of factors such as sediment depth, sediment type, water column height, tubing length, tubing diameter, and specific sediment characteristics like porosity and compaction and other sediment specific properties as described in subsequent paragraphs.

In the herein invention, sand sediment usually requires more pressure than mud and silt. Yet it should be noted that due to the relatively low depth of injection of the slurry or bio-paste within the sediment, and especially sediment sand, total pressure requirements are usually much lower than the one reported by land-based injection of slurry occurring in UIC wells or equivalent.

For instance, in shallow water environments with depths of 3 to 10, 10 to 50 meters and loose, granular sediment, injection pressures usually range from 2 to 60 bar in certain setting and 60 to 200 bars in other case. As sediment begins to compact in moderate depth environments of 50 to 200 meters, the necessary injection pressures might escalate to 200 to 500 bars especially in the case of clustered injection. In deeper-sea depth settings where depths exceed 200 meters and sediments are highly compacted or clay-rich, pressures could soar from 600 to 1200 bars or more.

The influence of water column height adds to the complexity, increasing pressure demands. A water column of 100 meters may necessitate pressures from 10 to 40 bars, while at 500 meters, required pressures could span a total injection requirement of 50 to 800 bars. At significant depths of 1000 meters, pressures might need to reach from 900 to 1500 bars to counteract the substantial hydrostatic pressure.

Tubing length and diameter critically influence the operational pressures within the injection system, particularly due to their impact on friction within the tubing. Longer tubing lengths, extending several hundred meters, inherently encounter increased friction, necessitating higher pressures, potentially ranging from 1 to over 60 bars in certain design, to maintain efficient slurry propulsion. Conversely, the diameter of the tubing plays a pivotal role in modulating this friction: narrower diameters increase friction, thus requiring higher pressures, whereas larger diameters decrease friction, enabling lower pressure requirements.

In this invention, larger tubing diameters are specifically favored, particularly when handling mud-based mediums. The use of larger tubes not only reduces the pressure requirements-often crucial for mud, which generally requires lower flow rate of injection and lower pressure—but also moderates the flow velocity. Controlling the flow velocity during the overall injection step is crucial and several flow velocities could be induced at several times during the injection process Injection time Inj(to) to Inj(tend) to maximize the injection and maximizing the chance of containment within the sediment matrix. This controlled flow velocity is critical, as it ensures the slurry is injected effectively without disrupting the sediment structure, thereby facilitating successful carbon storage. This approach optimizes both the energy efficiency of the system and the integrity of the carbon sequestration process, aligning with the operational goals of maximizing storage efficacy while minimizing environmental disturbance.

The configuration of the injection system, especially the number and arrangement of needles, critically affects pressure dynamics. A system managing 2-60 injector needles might operate effectively within 5 to 60 bar and 60 to 700 bars, whereas larger systems with 60 to 300 needles could demand a total of 800 to 1200 bars in certain embodiment to ensure uniform slurry distribution. These pressures can account either for the pressure requirements of a single needle injectors or as the overall pressure requirements of a needle bundle or needle cluster injector system.

It's important to differentiate between the maximum pressure capacity of the pump and the actual injection pressures used. While certain pumps may be capable of reaching up max pressures of 100 to 800 bars for single or low number of cluster injection; others, operating in large numbers of injection bundles or clustered injectors can be capable of reaching max pressure requirements from 800 bars to 1,400 bars of pressure. Operational pressures are typically set according to environmental conditions and specific injection requirements to ensure both efficacy and safety. This adaptability in pump design allows the technology to be efficiently applied across a broad range of geological and environmental conditions, optimizing injection efficacy while adhering to environmental compliance.

It is crucial to distinguish between the maximum pressure capacity of the pump at to referred to here as the initial sequence of time when the injection is launched and pressure peaks due to sediment rupture initiating the injection. Following this initial peak, there typically occurs a reduction in pressure to a stable baseline, which represents the ongoing injection pressure requirement.

Pumps utilized in this invention must be capable of delivering both the initial peak pressure at t0 and the sustained pressure necessary for the injection of multiple needles simultaneously. In certain embodiments, clustered injections are staged sequentially, such that peak pressures are achieved consecutively for each injector, while the cumulative injection pressure requirements for all active injectors establish a continuous baseline pressure throughout the duration of the injection. This design ensures efficient slurry propulsion and accurate sediment penetration, tailored to the specific geotechnical conditions of the injection site.

In a certain embodiment previously described in paragraph [V1 201], the location of the slurry injection will be within the deep-sea sediment near shore (5 to 1000 miles from the coast) at least 6 km from the shore, to generally meet international coastal injection standards. More precisely, operations are carried out at water column depth ranging from 1 to 50 m, 50 to 100 m, and 100 to 300 m, 300 to 1000 m. The selection of this specific location is determined by both the depth and the type of sediment, which must allow for the penetration of the needle. Additionally, the sediment properties must be suitable to facilitate the injection of the slurry at the required values, as described in previous paragraphs. The water column depth of 15 to 200 meters corresponds to the average range of depths for continental shelves worldwide and is optimal for the operation of this system. Reduced depths enhance the efficiency of the carbon removal methods described herein, as they lower the energy required by pumps to counteract hydrostatic pressure when these pumps are positioned on the injection vessel. This configuration is favored because it allows to achieve a lower emission-to-removal ratio scenario of this novel CDR method.

In various embodiments, the pressure demands of injection pumps vary significantly based on multiple factors. These factors include the depth at which the injection is taking place, the type of sediment, the height of the water column above the injection site, the length and diameter of the tubing, and specific characteristics of the sediment such as porosity or compaction. Additionally, the slurry composition also influences the required pressure.

The pressure required for effective injection can range from a minimal 5 bars in scenarios with shallow sediment depths and highly porous materials, escalating up to 1200 bars in conditions involving deeper sediment layers, denser or more compacted sediments, or higher water column pressures. The specific design of the injection system, including the number of needles and or injection apertures being utilized and their arrangement, further modifies the pressure needs. Each needle managed by a single pump might increase the cumulative pressure requirements, as the system must ensure consistent slurry delivery across all active injection points.

This variability necessitates a robust pump design capable of adapting to the dynamic pressure needs of the operation, ensuring efficient and safe slurry injection into marine sediments across a wide spectrum of environmental conditions.

### Description of Operation

Integration of Flexible Tubing and Corer Needle: The flexible tubing's connection to the corer needle is a meticulously designed assembly, ensuring a leak-proof and durable connection. The materials used for the tubing are carefully chosen to withstand the pressures and corrosive nature of the marine environment. This cohesive assembly ensures that there is no need for repeated disassembly, minimizing wear and tear. Only in specific cases, such as maintenance, repairs, or transitioning between vessels, would the assembly be disassembled, ensuring longevity and operational efficiency.

Deployment Mechanism (Refer to FIG. 4): The ship's onboard crane systems are fortified with robust mechanical structures, ensuring the corer needle's precise and controlled descent into the ocean. These systems are equipped with advanced sensors and feedback mechanisms that provide real-time data on the needle's position and orientation. In scenarios where cranes might not be ideal, alternative mechanisms like hoist systems or pulley arrangements can be used. These alternatives would be equipped with similar sensing and feedback systems to ensure accurate needle placement.

Ship Positioning and Stabilization: The ship's orientation and positioning are crucial during the injection operation. Advanced stabilizing systems, possibly including gyroscopic stabilizers, ensure the vessel remains steady, even in challenging marine conditions. The integration of advanced GPS tools or pingers provides real-time feedback on the ship's position, enabling quick corrections if any drift occurs. The ship's hull design and ballast systems also play a role in ensuring stability during these operations.

Depth of Needle Deployment: The corer needle's design allows for adjustable depth settings. The materials used in its construction are resistant to high pressures and corrosive marine environments. Its structure ensures optimal penetration, with specialized tips designed for varying sediment types. Sensors on the needle provide real-time data on its depth and orientation, ensuring accurate deployment.

Sediment Coring Weight Mechanism: The needle's design incorporates principles from sediment coring. The added weights, which are strategically placed, use gravitational force to aid in the needle's descent. These weights can be adjusted based on the sediment type and desired penetration depth. Side-mounted levelers, equipped with advanced sensors, guide the weights, ensuring optimal alignment and propulsion.

Depth of Needle Burial: The needle's penetration depth is adjustable, allowing for flexibility based on the sediment type and desired sequestration depth. Real-time feedback systems provide data on the needle's position, ensuring it reaches the desired depth accurately.

Activation of the Slurry Mixer and Pump: The onboard slurry mixer is equipped with advanced mixing blades and agitators, ensuring a consistent and homogenous slurry mixture. Sensors within the mixer provide data on the slurry's consistency, allowing for adjustments in real-time. The pumps are specifically designed to handle the slurry's viscosity and are equipped with pressure regulation systems to ensure optimal injection.

Pumping Pressure Considerations: The pump's design considers multiple factors to determine the optimal pressure. Advanced pressure regulation systems, combined with real-time feedback mechanisms, ensure the pump operates within the desired pressure range. The pump's materials are chosen to withstand high pressures and resist corrosion.

Fracture Formation in Sediments: The injection of the slurry results in fractures within the sediment. Advanced sensing systems provide real-time data on the fracture's shape and size, allowing for adjustments in the injection process if needed. The fracture's shape can vary based on sediment type, injection pressure, and slurry consistency. These fractures could take the form of an apple shape, extend horizontally, or manifest in an undefined shape. The water will diffuse through the sediment medium, while the organic bulk biomass will be retained and accumulate to form the fractured hole.

Formation Fracture Pressure: Advanced sensing systems provide data on the pressure required to initiate and propagate a hydraulic fracture. This data is used to adjust the pump's pressure in real-time, ensuring optimal penetration and spread of the slurry.

Post-Injection Maneuvers: After injection, advanced winch systems equipped with feedback mechanisms retrieve the corer needle. As the ship moves to the next injection point, advanced navigation systems ensure efficient and safe transit. The suspended needle's design ensures minimal drag, and its position can be adjusted based on real-time feedback systems. The ship's propulsion systems are optimized for these short transits, ensuring fuel efficiency and minimal environmental impact.

To further clarify and complete the ship positioning and stabilization systems previously described in paragraph [V1 146], a detailed list of the advanced positioning and stabilization mechanisms is provided to ensure optimal operational efficiency and precision during the injection process. Such systems include dynamic positioning, advanced GPS tools, pingers, gyroscopic stabilizers, and motion reference units. The dynamic positioning (DP) systems range from manual positioning without automated systems (DP Level 0) to fully redundant DP systems with three independent computer systems and backup power sources (DP Level 3). Key components of the DP system include position reference systems (PRS) utilizing advanced GPS tools and pingers, thrusters and propulsion units for fine-tuned control, gyroscopic stabilizers to reduce roll and pitch, wind sensors and motion reference units (MRU) for continuous monitoring, and a power management system (PMS) ensuring continuous power supply with redundancy. Operational protocols include pre-operation checks for comprehensive system calibration and monitoring. The scope of the above extends beyond the listed applications to include any future and novel innovations suitable for the purposes of this described invention.

The injection system described in paragraph [V1 171], [V1 196], [V1 197] and [V1 that using multiple needles operating further include advanced system of single pump to support multiple injection needles. This approach aims to optimize capital expenditure (CAPEX) and operational efficiency. The system described includes a central high-capacity pump, connected to a manifold system that distributes the slurry to multiple injection needles. The terminology encompassing this technology includes but is not limited to: centralized hydraulic power system, single-source hydraulic power unit, multi-well hydraulic control system, central pumping system, distributed hydraulic power network, single-pump manifold system, multiplex pump control system, multi-point injection system, centralized hydraulic distribution, shared hydraulic power system, multi-well injection manifold, single-point hydraulic drive, consolidated pump system, multi-station hydraulic network, centralized pumping station, multi-well hydraulic manifold, central hydraulic hub, single-source pump control, multi-injection point system, and centralized slurry injection system.

Operationally, these systems generally utilize an injection pump, such as a triplex displacement pump, as do all other pumps mentioned in this invention, where the number of pumps employed may range from one to ten, or more in certain specific injection vessel designs. The preferred implementation utilizes three to four pumps per injection vessel, each delivering biomass via three, four, or a plurality of needle injectors in a vertical, horizontal injection plane, or both orientations. Correspondingly, the number of needles associated with each pump is also variable, ranging from a minimum of two to a maximum of thirty needles per pump. Preferably, the system is designed to operate with three to fifteen needles per pump, although configurations may vary widely to include one up to 60 needles per pump as required by specific applications.

The integration of variable frequency drives (VFDs) within the system allows for dynamic control of pump operations, aligning with the real-time demands of the injection process. These VFDs are configured to match the varying combinations of needles and pumps, ranging from five to more than twenty-five, with an optimal range typically between twelve to twenty VFDs.

Additional components of the system include hydraulic hoses and fittings, which are engineered to withstand high pressures and the dynamic conditions of marine environments. Control and monitoring systems may be implemented in a singular or multiple setups depending on operational scale and complexity. Pressure regulators and flow meters are installed at each injection point to maintain precise control over pressure and to accurately measure flow rates, thereby ensuring efficiency and precision in system operations.

Using a single pump to handle multiple injector needles hinges on the precise coordination and substantial power output of the pump to manage the sequential injection through various needles. This system operates under a configuration where each injector needle, or each injector needle aperture, or vertical needle aperture set, as well as horizontal aperture set, is activated in stages, with intervals ranging from 1 to 3 seconds, 3 to 5 seconds, 5 to 15 seconds, and 1 second to 1 minute, depending on the time required for the displacement of the sediment at the to interval, as well as the sediment type and local condition. This staging allows for a controlled and gradual increase in operational demand on the pump.

Variable frequency drives (VFDs) are typically integrated to modulate the pump speed and output according to real-time data, ensuring each needle operates optimally within its designated time slot. This system not only maximizes the efficiency of the injection process but also helps in maintaining the structural integrity of the seabed by avoiding excessive pressure buildup which could lead to sediment disruption. By employing a single pump for multiple needles, the system benefits from reduced capital and operational expenditures while enhancing the ability to scale up or modify the injection parameters based on situational requirements. The strategic interval setting between needle activations is pivotal in managing the cumulative load on the pump, ensuring it delivers consistent performance throughout the injection cycle. In the present design, it is preferable to have interval times between each initialization of needle as small as possible as to increase the efficiency of the system of simultaneous injection described in the herein invention.

This enhanced multi-needle injection system capitalizes on advanced technologies to significantly reduce capital expenditures (CAPEX) and elevate operational efficiencies in offshore carbon injection applications. By adopting and modifying established methodologies from the oil and gas industry, this system provides a robust and economically viable solution for carbon sequestration efforts.

In certain embodiments, the quantity of slurry that can be injected at each injection point within the sediment, and the spacing between each needle injector, are definitively established using methods of computational fluid dynamics. CFD models are employed to accurately predict the height of the slurry injection propagation within the sediment. These metrics are established through preparative work and pre-assessment of the locations, potentials, types of sediments, and operation predictions. In other case these processes are integrated as part of the sensor automation step, described in other sections of this document, occurring just before the injection steps, after penetration, or as part of the ongoing automated MRV (Monitoring, Reporting, and Verification) processes during or after the operation.

The domain for CFD analysis is defined by an inner radius Rₒ, an outer radius R₁, and a height H. The model characterizes the porous medium using parameters such as Young's modulus E, Poisson's ratio v, fracture toughness Kⱼₑ, porosity ρ, Biot's coefficient α, Biot's modulus M, and hydraulic conductivity k. An incompressible Newtonian fluid with a viscosity f is injected at a constant rate Q₀ into the center of a fracture from a vertical wellbore. The model seeks to determine the fracture aperture w(r,t), net pressure p(r,t) (defined as the difference between the fracturing fluid pressure p_{f}(r,t), and the confining stress σ_{g}, and the fracture radius R(t). In this example, the fracture is modeled as horizontal, circle-shaped, and planar within a cylindrical domain, employing a radial or “Penny-Shaped” model.

For the simulation, injection occurs 20 meters withing the sediment. The model incorporates gravity to account for the gradient in soil density, thereby creating a stress distribution that reflects changes in soil density with depth. Notably, the relationship between density and depth in saturated seabed soil is not linear but tends to stabilize with increasing depth due to compaction and consolidation. Unlike conventional hydraulic fracking models in the oil industry, this model considers the gradient in density.

In this exemplary embodiment, the saturated soil properties are specified as follows: Young's modulus E=3 MPa, Poisson's ratio ν=0.4, Biot's modulus 0.687 MPa, hydraulic conductivity 9.8×10⁻⁹ m/s, porosity 0.8, and pore fluid specific weight 9.8 kPa/m. The fracture properties include cohesive energy 0.120 Pa m, cohesive strength 1.25 kPa, and cohesive stiffness 1.7×10³ kPa/m, with a Biot's Coefficient of 0.75. The slurry viscosity is 30 cP with an injection rate of 0.5 m3/min.

The fracture propagation resulting from this configuration is predominantly planar with a radius of 15 meters and a minimal thickness of 5.2 centimeters at a depth of 200 m. The peak pressure required for this injection rate is 5.5 MPa or 55 bar above ambient pressure. Adjustments in the injection flow rate to 1.5 m3/min result in a peak pressure of 7.1 MPa or 71 bar. At 1000 m depth, the total pressures required are approximately 170 bar, respectively, highlighting the significant pressures involved in deep-sea operations.

While the described propagation shape is planar and circular, it is imperative to note that sediment characteristics can influence the shape of fracture propagation. This section aims to provide comprehensive insights into the technology's operation but does not limit its application to the specific systems described. Other feasible fracture shapes include radial, elliptical, branched, and networked fractures, which may also be relevant depending on the sediment composition and operational conditions. These variations illustrate the technology's adaptability to diverse geological scenarios, ensuring that the scope of the invention is fully understood but not restricted by the examples provided.

### Zone of Injection

Suitable Seabed Sediments: The success of the injection process relies heavily on the type of seabed sediment available. Calcareous sediments, siliceous sediments, deep-sea clay, glacial sediment, terrigenous sediments, and ocean margin sediments are considered ideal for this operation. These sediment types offer the required consistency and compactness to retain the injected slurry, ensuring that it doesn't resurface.

Sediment Column Volume: The depth and volume of the sediment column are essential. It must have a capacity vast enough to accommodate the injected slurry without allowing it to resurface. If the slurry were to rise back into the water column, it would negate the carbon sequestration process, making the operation ineffective.

Detailed Sediment Characteristics: In specific zones, turbidites from the continental rise can be found in sediment layers. These turbidites can have a fine-grained texture at the top, with silt present at the bottom layers, often spanning up to +10 m. Marly oozes are another sediment type that commonly contains a blend of silt, carbonate, clay minerals, and a small percentage of organic carbon. In certain layers, especially those with turbidites, the carbonate content is higher, contributing to the sediment's overall density and compactness. These unique sediment characteristics play a crucial role in determining the success rate of the injection process.

Grain Size and Permeability: Grain size in these sediments can vary significantly, with some layers having grain sizes less than 4 μm, resulting in low permeabilities. The first 25 m below the seabed might have permeabilities ranging from 5 to 12 cm s\hat{ }−1. In contrast, below 25 m, the permeability can be as low as 10\hat{ }−10 cm s\hat{ }−1. However, in carbonate oozes, permeabilities have been observed to be around 10\hat{ }−6 cm s\hat{ }−1. This variation in permeability plays a pivotal role in determining the dispersion and retention of the injected slurry.

Bioturbation and Animal Disturbance: The uppermost layers of the sediment, typically spanning the first 10 m, are prone to bioturbation. This disturbance, caused by marine animals, can affect the sediment structure. Infilled burrows or mottling can be observed in over 50% of these layers due to this disturbance. Bioturbation can alter the sediment's consistency, which, in turn, can influence the injection process.

Abyssal Plain and Abyssal Hill Environments: These environments have unique sediment characteristics that can be leveraged for the injection operation. The abyssal plain, for instance, can have sediment types like sandy silt, silt, sand-silt-clay, clayey silt, silty clay, and clay, with densities ranging from 1.352 g/cm\hat{ }3 to 1.652 g/cm\hat{ }3 and porosities as high as 80%. The velocity ratios in these sediments can also vary, influencing the sediment's overall consistency and compactness.

Arrival and Positioning of the Ship: Once the ship reaches the designated injection zone, it utilizes advanced navigation systems to ensure precise positioning. Leveraging real-time data, the ship positions itself optimally to begin the injection process. This accurate positioning is essential to ensure the needle's correct deployment, optimizing the injection's success rate.

In other embodiments of the marine carbon dioxide removal system described herein, the types of seabed sediments where injection can occur are extensive and varied. These include clay, silty clay, clayey silt, silty sand, sandy silt, fine sand, medium sand, coarse sand, mixed sand and gravel, gravelly sand, fine gravel, medium gravel, coarse gravel, peaty mud, organic mud, calcarenite, bioclastic sand, oolitic sand, shell debris, coral fragments, volcanic ash, siliceous ooze, calcareous ooze, radiolarian ooze, diatomaceous ooze, chalk, marl, diatomite, pumice, glacial till, glaciomarine clay, dropstone-containing sediment, hydrothermal vent sediments, manganese nodule pavements, phosphorite nodules, methane hydrate-bearing sediments, cold seep communities, carbonate crusts, pelagic red clays, contourite deposits, turbidite sequences, hemipelagic muds, sapropel layers, hydrocarbon seep areas, and brine pool surroundings. This comprehensive array of sediment types emphasizes the adaptable nature of the injection technology to function effectively across a diverse range of geological and biological environments. The inclusion of such a wide spectrum of sediment types does not limit the scope of the invention; rather, it serves to elucidate the potential applications and versatility of the technology, ensuring that stakeholders and implementers appreciate the breadth of environments suitable for this carbon sequestration method.

### Injection Process

Flow Rate of Injection: The flow rate during the injection process is a crucial factor determining the efficiency and success of the operation. The pump's capacity, combined with the slurry's consistency, dictates the injection's flow rate. In some embodiments, the flow rate can range from as low as 0.01 m³ of slurry per minute to as high as 6 m³ per minute. In other instances, especially with more robust pumping systems, the flow rate can range from 6 to 20 m³ per minute, or even from 20 to 50 m³ per minute. In specific scenarios where impact injection is employed, the injection can be rapid, with quantities ranging from 0.01 m³ to 3 m³ per min, or in larger operations, from 3 m³ to 10 m³ per min and 10 m³ to 50 m³.

Duration of Injection: The total time taken for the injection process is influenced by several factors, including the pump's characteristics, the sediment's nature, the depth of injection, the resistance offered by the pipe, the pump's nominal power, the number of pumps in operation, and the size of the vessel. Generally, the injection process can range from a quick 1-5 minutes for smaller operations. For medium-sized operations, the time can range from 5 to 15 minutes. In larger operations, where vast quantities of slurry are injected, the process can take anywhere from 15 minutes to 60 minutes. In some of the most extensive operations, the injection time can extend from 60 minutes to 180 minutes.

Corer Needle Penetration and Lifting Time: The time taken for the corer needle to penetrate the seabed and subsequently be lifted post-injection is pivotal for the operation's efficiency. Several factors, such as the sediment's nature, depth of operation, and the weight mechanism of the corer needle, influence this duration. Typically, the entire process, which includes both penetration and lifting, can take anywhere from 1 to 3 minutes for smaller operations. For medium-sized operations, this duration can range from 6 to 15 minutes. For larger operations, especially those involving deep-sea sediments and substantial slurry quantities, the process can take 15 to 45 minutes. It's worth noting that this duration encompasses the pump's activation time, the time taken to close the pump system when it's not active, the duration for the winch system to lift and drop the corer needle, and the time taken by the weight mechanism to ensure optimal needle penetration.

The duration of the injection process further varies depending on the volume of slurry injected and the flow rate employed. Each injection point within the needle is capable of injecting between 1 to 30,000 cubic meters of slurry from one embodiment to another. The overall injection duration per injection vessel site and or per injection needle can further range from 180 minutes up to 30,000 minutes.

### Scientific Basis For Carbon Sequestration Geological Storage

Deep-sea Sediment Sequestration: One of the primary advantages of burying organic matter in deep-sea sediments is that it places the carbon-rich material away from biological interactions. This deep burial minimizes the risk of rapid remineralization into organic compounds. Additionally, it prevents the resuspension of the carbon in the water column, which could eventually lead it to return to the surface and subsequently, the atmosphere, driven by the thermohaline circulation. The deeper the burial, the more removed the organic matter is from such risks.

Benefits of Biomass Encapsulation: Encapsulating the biomass and burying it deep below the sediment in abyssal plains effectively ensures the permanent sequestration of carbon. This encapsulation technique provides a physical barrier, shielding the biomass from potential grazers such as macrofauna and meiofauna. The threat from these grazers is already relatively low due to the sparse total biomass found at such depths (Chih-Lin Wei et al., 2010). Moreover, the extreme conditions of the deep sea, characterized by cold temperatures and high pressures, significantly hinder bacterial enzymatic processes, leading to much slower degradation rates of the encapsulated biomass (Turley CM, 2000; de Jesus Mendes et al, 2007). This biogeomimetic approach, as delineated in this invention, offers added protection to the organic matter exuding from the biomass, safeguarding it from bacterial degradation (Hedges et al, 2001; H Cheng, et al, 2012).

Carbon Trapping Mechanisms: In the unlikely event that some CO2CO₂ forms as a result of biomass degradation, it doesn't pose a threat to the environment. Instead, this CO₂ CO₂ gets trapped within clathrates for example hydrates, preventing it from dissolving into the water column (Qureshi et al, 2022). The method described in this patent leverages a sustainable and environmentally-friendly approach. It enhances natural oceanic processes and exploits the inherent capability of algae to absorb CO2CO₂, all the while avoiding the introduction of foreign substances into the seabed. When the naturally occurring algae are buried below a depth of 1,000 m, the carbon within their biomass remains sequestered over geological timescales, owing to the slow remineralization of organic carbon to its inorganic form. Clatharate formation is also a process observed for methane.

Advantages over Other CDR Methods: Burying organic carbon approximately 10 cm below the seabed ensures that it remains isolated from the oxygen zone and benthic biota. This method represents a superior carbon dioxide removal (CDR) technique, bypassing the usual pitfalls like disturbing ecosystem dynamics or inadvertently releasing carbon back to the surface, which other CDR methods might encounter. The processes and advantages detailed in this patent promise remarkably prolonged sequestration periods, potentially extending to millions of years. This ensures that the carbon remains locked away, far removed from the atmosphere, contributing significantly to climate change mitigation.

Hydrate formation is a crucial aspect of deep-sea carbon sequestration. Clathrate hydrates, also known as gas hydrates, are crystalline ice-like substances formed when water molecules create a cage-like structure that traps gas molecules, including CO2CO₂ and methane. In deep-sea environments, where temperatures are low and pressures are high, hydrates can form at depths below 800 meters for CH4 and 300 m for CO₂. These conditions allow the CO₂ generated from potential biomass degradation to become trapped within hydrates, preventing it from dissolving into the water column or returning to the atmosphere. In our specific process, the burial of organic matter in deep-sea sediments enhances natural clathrate formation, capitalizing on the unique geological conditions found in the abyssal plains. This approach not only secures the sequestration of carbon but also aligns with sustainable and environmentally friendly practices, utilizing natural oceanic processes to combat climate change.

Understanding geological processes like diagenesis, catagenesis, and metagenesis is essential in the context of deep-sea carbon sequestration. Diagenesis refers to the physical and chemical changes that sediment undergoes after deposition, often leading to lithification. Catagenesis is the second stage of coalification and involves the thermal breakdown of organic matter into oil and gas. Metagenesis, the final stage, leads to the formation of graphite or even diamond in extreme conditions. In our specific process, diagenesis would facilitate the encapsulation of biomass, creating physical barriers that shield it from degradation. Catagenesis and metagenesis further ensure that even if some breakdown occurs, the resulting products are stable and remain sequestered within the deep-sea sediments. These geological phenomena contribute to the efficiency and permanence of carbon sequestration in this innovative approach.

The present embodiment offers significant advancements over previous innovation, particularly in addressing the issue of resuspension. In previous methods, the use of penetrators could lead to the resuspension of carbon in the water column, posing a risk of it returning to the surface and atmosphere. However, the current approach, with its emphasis on deep burial and encapsulation, minimizes this risk. By burying organic carbon approximately 10 cm below the seabed and isolating it from oxygen and benthic biota, this method ensures that the carbon remains locked away for potentially millions of years. The introduction of a circular multi-micro injection system, with simultaneous injections through multiple needles, further enhances the efficiency of the process. This embodiment represents a superior carbon dioxide removal technique that not only overcomes previous challenges but also sets new standards in prolonged sequestration and environmental sustainability.

The carbon sequestration technology developed under this invention operates by accelerating the natural storage of fixed carbon within marine sediments. Leveraging the ocean's biological pump, which effectively captures atmospheric CO₂ via phytoplankton, this technology enhances the process whereby a minor fraction (approximately 1%) of captured carbon reaches deep-sea sediments. Given that oceans cover 70% of the Earth's surface, this biological pump plays a pivotal role in global carbon storage.

The method involves sub-sediment storage of residual biomass within anoxic layers of marine sediments, ensuring carbon sequestration for millennia. During the initial stages of biomass degradation, oxygen serves as the primary electron acceptor, facilitating the oxidation of organic carbon due to its high energy yield (see Table 3 below). Once the available oxygen is depleted, the degradation process transitions through a series of less energetically favorable electron acceptors, as part of a redox cascade. Known to those skilled in the art, this cascade significantly slows down the degradation of organic matter, particularly for complex macromolecules such as lignocellulosic biomass.

Globally, it is estimated that between 150 to 200 billion tons of organic carbon are stored within the upper layers of marine sediments. Introducing biomass directly into later stages of the redox cascade—where oxygen is absent-ensures that carbon is preserved for extended time periods. This preservation mechanism is analogous to the phenomenon observed in ‘bog bodies’, where human remains have been naturally mummified in anoxic and nutrient-rich peat bogs over millennia.

This invention delineates a process that not only mimics but enhances these natural geological and biochemical pathways to sequester carbon efficiently and sustainably. By a disruption of the REDOX cascade, embedding biomass in environments where the conditions are unfavorable for rapid decomposition, the technology locks away carbon, thus contributing significantly to the mitigation of anthropogenic carbon emissions.

TABLE 3
Description of sediment electron acceptors, their typical reaction
rates and environmental concentrations in marine sediments.

| | | | Depth in | Energy | | | |
|---|---|---|---|---|---|---|---|
| | Depth in | | sediment at | required | Rate of | | |
| | sediment at | | 1000 m+ | (ΔG, | reaction | | |
| Electron | 200 m water | | water depth | kJ/mol | (mmol/m2 | | |
| acceptor | depth (cm) | | (m) | CH2O) | day) | Concentration | |
| Oxygen | 0-10 | | 0-1 | −479 | 1-10 | 200 | uM |
| Nitrate | 10-20 | | 1-2 | −453 | 0-1 | 50 | uM |
| Iron (III) oxides | 20-60 | | 2-3 | −114 | 1-5 | 100 | mmol/kg |
| Mn (III) oxides | 20-30 | | 3-4 | −349 | 0-1 | 100 | mmol/kg |
| Sulfate | 60-5 | m | 4-20 m | −77 | 1e−9-1e−6 | 28 | mM |
| CO2/Acetate | 5 | m+ | | −28 | 1e−9-1e−6 | 3-50 | mM |
| (Methanogenesis) | | | | | | | |

Sub-sediment carbon storage is targeted at water depths ranging from 5 to 1500 meters, with injection occurring at a target depth of approximatively 15 meters within the sediment to optimize environmental parameters influencing organic carbon degradation in marine sediments. Operations are conducted offshore in coastal areas to minimize transportation costs and emissions, with biomass sourced from adjacent coastal regions. This approach mimics environmental parameters conducive to the long-term storage of woody (ligno-cellulosic biomass) observed in the Bengal Fan, characterized by limited oxygen exposure time (OET) and sediment compaction.

Upon injection, the remnant biomass slurry becomes embedded within the sediment matrix, creating a denser area of biomass compared to the surrounding sediment. This process displaces slurry water into adjacent sediment areas, along with a small percentage of biomass, resulting in the formation of a dense corer of biomass surrounded by sediment, thereby minimizing solute transport within the biomass. This process emulates the compaction observed in the Bengal Fan due to high sedimentation rates, effectively preserving ligno-cellulosic biomass for extended periods.

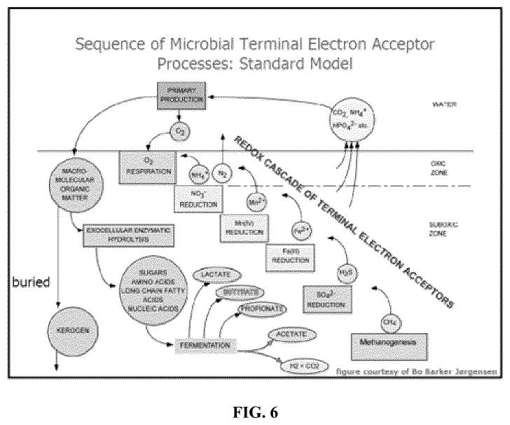

The initial stage of biomass degradation involves breaking down accessible and easily consumable biomass molecules through extracellular enzymatic hydrolysis, facilitated by enzymes produced by aerobic microorganisms to liberate small organic molecules (FIG. 6). Subsequently, anaerobic fermentation occurs to produce readily utilizable carbon substrates for the redox cascade (FIG. 6). Adequate OET is required to initiate enzymatic hydrolysis of biomass, with longer OET necessary for initiating degradation of ligno-cellulosic biomass compared to algal biomass. Insufficient OET impedes degradation initiation and subsequent progression through the redox cascade due to the heightened energetic requirements of anoxic degradation processes.

This invention′ biogeomimetic approach draws inspiration from the natural process of carbon storage in marine sediments, as demonstrated by 2019 research in the Bengal Fan highlighting the sustained burial and preservation of woody debris over millions of years. The Bengal Fan, influenced by high sedimentation rates and reduced oxygen exposure time, exhibits limited solute diffusion necessary for biomass degradation, resulting in a high degree of organic carbon preservation. This rapid deposition and burial effectively prevent organic carbon from undergoing degradation through the redox cascade due to limited microbial access-both physically and energetically. The invention present a safe, permanent, and scalable carbon removal solution by controlling diffusive transport and OET, facilitating permanent carbon removal across a spectrum of sediment geochemistries.

The worst-case scenario of this novel path of CDR establishes that 1% of injected carbon is released into the overlying water column, which is in line with or lower than current rates of dissolved inorganic carbon fluxes out of marine sediments.

Over the past 40 years, scientists have been studying the fate of organic carbon in the marine environment and the factors controlling its degradation and preservation. In anoxic sediments, organic carbon degradation proceeds at a slow rate due to multiple factors and environmental inhibitors such as low substrate availability, low temperature, competing microbial and abiotic reactions, mineral protection, and inaccessibility of molecularly complex organic carbon that has evaded toxic degradation.

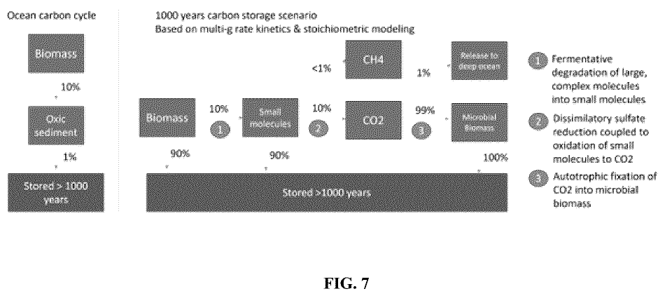

Using biogeochemical knowledge about marine sediments and the microbial and abiotic processes occurring therein, a degradation model was generated to evaluate the worst-case scenario for biomass degradation and release under environmental conditions in deep sediment (FIG. 6). It has been determined that, at maximum, 10% of the biomass could be remineralized over 1000 years, releasing CO₂ or CH4 depending on the water depth of deployment and biogeochemical zonation of relevant reactions (see FIG. 7). The exact fate of this 10% of remineralized gas is unknown, but based on well-established laboratory and in situ studies, the fate of released CO₂ and CH4 in operating locations can be predicted based on existing environmental data and models. Ultimately, only 1% of the released CO₂ or CH4 is expected to escape the marine sediments via diffusion, leading to stable long-term storage of carbon. For any carbon leakage to occur, it would need to diffuse up 15 m out of the sediment, a process which will take at minimum 225 years due to the nature of solute diffusion in sediments (0.06 m/yr diffusion maximum). Over 1000+ years, marine sediments at depths of 15 m+ become increasingly compacted, decreasing diffusion and transfer of solutes and substrates necessary for microbial biogeochemical transformation of biomass.

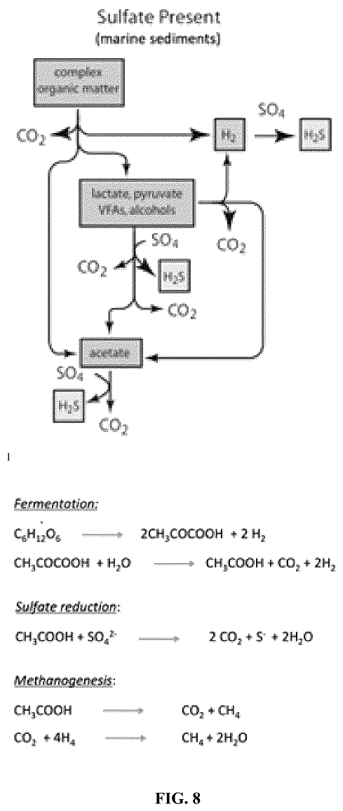

Based on existing studies of degradation of remnant agricultural biomass and plankton biomass, the rate of degradation slows exponentially over time and is up to 1000× slower in anoxic sediments compared to oxic sediments globally. After the liberation of small organic molecules, sulfate reduction is the dominant form of organic carbon degradation in the deep sediment, accounting for 50% of total biomass degradation in global sediments. Any released carbon would be subject to this dominant but slow and energetically unfavorable process, which generates CO₂ and H₂S. as previously described in paragraph [V1 167], CO₂ will form hydrates at 10+m deep in marine sediments at <10° C., and otherwise will become a substrate for chemoautotrophic microorganisms living in sediments. H₂S is toxic to marine organisms but is quickly abiotically oxidized to SO₄ in the presence of oxygen, preventing toxicity to marine organisms. 10-20% of H₂S generated will form pyrite (FeS) by reacting with Fe hydrates, further stabilizing the organic matter due to favorable organo-mineral reactions (see FIG. 8). Methanogenesis consumes 15% of the CO₂ produced by sulfate oxidation. Over time, porosity is decreased by compaction and sedimentation, slowing diffusion which allows for additional FeS formation and slows CO₂ and methane diffusion, allowing these substrates to be utilized by other sediment microorganisms or to form clathrates.

Following depletion of sulfate in the surrounding sediment and biomass pore space, methanogenesis will dominate. At depths of 20 to 200 m, sulfate will be rapidly repleted because slurry is being injected into the methanogenesis zone with no measurable sulfate present, and the biomass slurry brings with it minimal amounts of sulfate. At depths of 1000-1500 m, sulfate is not depleted and will be the dominant process until the substrate is depleted. Methanogenesis proceeds via hydrogenotrophic or acetoclastic mechanisms, and over an 18 m sediment corer, has been demonstrated to dramatically slow with increasing depth and decreasing temperature. Methane hydrates form at lower temperatures and higher pressures than CO₂ hydrates and are a globally important storage mechanism for carbon. Methane hydrates form in zones with sufficient rates of methanogenesis and sedimentation (30 cm kyr−1) and at diffusive interfaces, which are expected to dominate in injection settings.

Addition of biomass above natural levels perturbs the sediment system, but micro-injection technology ensures that new and already stored biomass will not be re-suspended into the toxic layer or disturb the seabed and benthic organisms. This ensures effective storage of biomass and prevents harmful impacts on benthic organisms. Biomass slurry is mixed together with mechanically-ground remnant biomass and marine water to achieve optimal physical parameters for microinjection. The low levels of oxygen in the biomass slurry will quickly be depleted due to toxic degradation of biomass, releasing less than 0.01%. Existing marine carbon dioxide removal technologies like seaweed sinking release 70% of the carbon captured by photosynthesis and perturb benthic ecosystems by introducing biomass in the oxygenated top of the sediment, de-oxygenating their habitat. Biomass injection is more energetically intensive than biomass sinking, however, it avoids ecosystem impacts (like deoxygenation) and limitations associated with sinking biomass in localized hypoxic zones, which generates methane in the water column.

In certain embodiment, the carbon sequestration potential of the herein described invention is further enhanced. This new embodiment involves the injection of the biomass slurry with specific bacteria designed to facilitate controlled chemical reactions during the degradation of the biomass with the aim to further limit any potential degradation of the biomass into CO₂, CH4 or unwanted gas form that could result in a leak back of carbon to the seabed. In this embodiment, bacteria (referred to as “Bacteria XYZ”) are introduced into the biomass slurry at predetermined concentrations ranging from 0.01% to 30% of the total slurry composition. These bacteria are further introduced at specific depth withing the sediment and or at specific time in the injection operation. The purpose of these bacteria is to enable and steer specific chemical reactions in a controlled manner, thereby optimizing the degradation process and enhancing the sequestration efficiency. Examples of such controlled reactions include:
1. Example One: Bacteria X promotes the anaerobic degradation of biomass, leading to the formation of stable organic compounds that are less prone to remineralization.
2. Example Two: Bacteria Y facilitates the rapid conversion of organic carbon into biochar, which is highly resistant to microbial degradation.
3. Example Three: Bacteria Z enhances the formation of carbonates by accelerating the reaction between organic carbon and available minerals in the sediment. The introduction of these bacteria ensures that the degradation pathway of the biomass is directed towards the formation of more stable carbon compounds, thereby increasing the longevity and stability of the sequestered carbon.

Another embodiment, as an iteration of the above-described system, focuses on slurry methanogenesis mitigation. This iteration is particularly relevant for slurry injection on land for type V wells or similar, as well as related applications but is also suitable for the herein system of microinjection. In this iteration, torrefied biomass is used partially or entirely instead of raw organic biomass as described in the detailed description of the herein invention. In this embodiment, the torrefied process is integrated directly on the injection vessel prior to injection or on land prior to its transportation via seaways.

Additionally, a clay mixture can be added to either raw or torrefied biomass for the injection process. The slurry composition can also consist solely of the clay mixture and biomass with the purpose of further mitigating bacterial action and degradation. The concentration of clay in the slurry ranges as follows: 1 to 10% in one iteration, 10% to 25% in another iteration, and 25% to 50% in the last iteration. The clay is obtained from: a) a nearby dredging operations and serves as recycle and repurposing material, or b) via an integrated system of dredging installed on the injection vessel, or c) via an integrated system of a suction pump directly installed on the injection sea vessel, or finally d) using the same injection mechanism described herein. The clay is then brought back to the mixer present on the injection vessel, where it is mixed with seawater and organic material to be further re-injected into the sediment. The purpose of this iteration is to limit any further degradation of the biomass by 1) limiting the mobility of microbes to degrade the biomass, and 2) limiting the diffusive transport of any CO₂ gas formed via degradation, overall increasing its conservation and storage of the organic carbon for millions of years, and further lowering the uncertainty of carbon resurfacing to the seabed, ocean, or atmosphere centuries later.

The method described herein ensures the long-term and safe sequestration of atmospheric carbon within marine sediments by leveraging a precisely controlled sequence of natural processes. This succession of processes, which can be viewed as a formula for carbon storage, is the cornerstone of the technology, guaranteeing that the injected carbon remains securely stored over geological timescales.
i. Deep Burial Initiation: The first step in this sequence involves the injection of a carefully formulated slurry composed of ligno-cellulosic biomass and seawater into deep-sea sediments. The slurry is injected at targeted depths, where it becomes encapsulated within the sediment matrix. This deep burial is essential, as it isolates the biomass from the surrounding environment, effectively initiating the carbon sequestration process.
ii. Anoxic Encapsulation: Following burial, the injected biomass is positioned within an anoxic layer of marine sediment. This strategic placement prevents aerobic degradation and initiates the natural preservation process. The anoxic conditions are critical, as they inhibit the breakdown of organic carbon, ensuring that the carbon remains stable and unreactive.
iii. Controlled Oxygen Exposure Time (OET): The degradation of organic carbon within the sediment is precisely managed through controlled exposure to oxygen. By carefully regulating the Oxygen Exposure Time (OET), the method ensures that the redox cascade progresses slowly. This controlled progression is crucial, as it favors the preservation of complex organic molecules, such as lignin, which are key to long-term carbon stability.
iv. Redox Cascade Progression: As oxygen is depleted within the sediment, the method ensures a transition through a series of less energetically favorable electron acceptors. These include nitrate, iron (III) oxides, manganese oxides, and sulfate, with the process ultimately leading to methanogenesis. This step is vital for stabilizing the carbon within the sediment, as it slows down the degradation process significantly, ensuring that the carbon remains securely stored.
v. Formation of Stable Carbon Compounds: Throughout the redox cascade, the method actively promotes the formation of stable carbon compounds. These include methane and CO2 hydrates, which are naturally trapped within the sediment. The formation of these compounds is essential, as it prevents the release of carbon into the water column or atmosphere, thereby ensuring its long-term sequestration.
vi. Sediment Compaction and Limited Diffusion: The method induces sediment compaction around the injected biomass. This compaction plays a pivotal role in limiting the diffusion of solutes and substrates, which further stabilizes the carbon. By reducing the mobility of these substances, the method ensures that the carbon remains locked within the sediment matrix, protected from potential degradation.
vii. Microbial Interactions and Controlled Reactions: To further enhance the stability of the sequestered carbon, an alternative approach involves specific microbial communities or chemical additives into the slurry. These elements facilitate controlled chemical reactions within the sediment, promoting the conversion of organic carbon into highly stable forms, such as biochar or carbonates. This step enhances the longevity of carbon storage, making it more resistant to degradation.
viii. Final Carbon Stabilization: The sequence culminates in the final stabilization of carbon through the formation of mineral-organic complexes and hydrates within the sediment. These stable compounds, formed under carefully controlled conditions, ensure that the carbon remains sequestered over geological timescales, providing a secure and permanent solution for atmospheric carbon storage.

The innovative technology and engineering design described in this invention play a critical role in enabling and enhancing the targeted natural processes that facilitate long-term and secure carbon sequestration within marine sediments. The success of this carbon dioxide removal method is achieved through the strategic integration of engineered systems with natural processes that govern carbon preservation in the deep-sea environment.

The core needle injection system is central to the technology, designed to precisely deliver the ligno-cellulosic biomass slurry deep into the seabed. This engineered approach ensures that the biomass is immediately encapsulated within the sediment matrix, isolating it from oxygen and benthic biota. This process directly induces the natural mechanism of deep burial and encapsulation, which is essential for preventing carbon resuspension and ensuring long-term sequestration. The effectiveness of this process is a direct result of the precise depth control and placement capabilities of the injection system.

The system enhances the natural biological pump—a key driver of carbon transport to deep-sea sediments—by carefully preparing and injecting a biomass slurry optimized for long-term storage. The controlled formation of this slurry, coupled with the use of a flexible pipe system, ensures that a higher fraction of atmospheric carbon captured by the biological pump is sequestered deep within marine sediments for long-term storage. This engineered process significantly amplifies the natural carbon storage capacity of the ocean.

The targeted injection of biomass at specific depths within anoxic sediment layers is facilitated by advanced depth control mechanisms. By placing the biomass within these layers, where oxygen is limited or absent, the system prevents aerobic degradation and initiates the natural preservation processes that lead to long-term carbon storage. The engineered system's ability to maintain controlled oxygen exposure time (OET) is essential for managing the redox cascade and preserving the carbon over millennia.

The system's ability to select optimal sediment depths and types, combined with the controlled compaction of the injected biomass, mimics and enhances natural preservation mechanisms observed in environments like the Bengal Fan. By embedding the biomass in these carefully chosen conditions, the system slows down degradation processes, ensuring the carbon remains stable and preserved for millions of years. The use of sediment compaction techniques further reduces solute transport, creating a stable environment that minimizes the risk of carbon release.

The engineered system is designed to optimize the environmental parameters that influence carbon degradation and preservation. The precise selection of injection depths, ranging from 5 to 1500 meters, and the adjustment of injection pressures and slurry composition are all tailored to reduce degradation rates and enhance the stability of stored carbon. This method ensures that the injected carbon is sequestered in conditions that naturally favor long-term storage.

The management of oxygen levels within the slurry is a key aspect of the engineered system. By controlling the initial oxygen exposure, the system ensures that the redox cascade progresses slowly, preserving complex organic molecules like lignin. This controlled degradation process, facilitated by the engineering design, is essential for achieving the long-term stability of the sequestered carbon.

The deep burial techniques employed by the system are crucial for maintaining low diffusion rates, which are necessary to keep the carbon sequestered within the sediment. The controlled injection pressure and depth ensure that the biomass remains buried in a stable environment, further reducing the risk of carbon release.

The system leverages natural microbial processes such as sulfate reduction and methanogenesis by carefully targeting specific sediment depths for injection. These processes, which proceed slowly in anoxic conditions, are enhanced by the engineered system, resulting in the stabilization of carbon within the sediment. The introduction of specific microbial communities or chemical additives into the slurry further controls these processes, promoting the formation of stable carbon compounds.

The culmination of these engineered processes is the final stabilization of carbon within the sediment. The system's ability to induce the formation of mineral-organic complexes and hydrates under controlled conditions ensures that the carbon is securely sequestered over geological timescales. This stable storage is the ultimate goal of the system, achieved through the precise interaction between engineered technologies and natural processes.

### Carbon Storage Capacity

Storage capacity depends on the pump the slurry characteristic the depth of injection the characteristics of the biomass the setup of the pumps and type of pumps.

In certain embodiment, a slurry volume=20 m³ and stores between 15 to 70 t CO₂ equivalent.

In certain embodiment, a slurry volume=40 m³ and stores between 20 to 150 t CO₂ equivalent.

The carbon storage capacity of a fracture hole is mostly defined by its volume

MRV (Monitoring, Reporting, and Value Verifications)

MRV, an abbreviation for Monitoring, Reporting, and Value Verifications, is an integral component of the Carbon Dioxide Removal (CDR) methods described in this invention. Probes for Monitoring: In certain embodiments, the MRV system-which entails measuring and tracking carbon, alongside environmental assessments of the technology—is executed using probes. These probes are strategically positioned: on top of, below, on the side, and even within each borehole slurry fracture. These probes serve as the eyes and ears of the system, constantly monitoring the conditions and ensuring that the carbon sequestration process is proceeding as planned.

Visual Clues and Deep-Sea Monitoring: The process may also rely on visual clues to validate that the injection is successful. This can be achieved by setting up video equipment and deep-sea torches to study the resuspension and any other pertinent visual indicators. By directly observing the injection site, operators can gain immediate insights into the process, allowing for real-time adjustments and verifications.

Laboratory Testing: For the technology detailed herein, MRV tests will be conducted in a pressure laboratory to emulate realistic oceanic conditions. This approach not only ensures that the technology's efficiency is assessed under conditions that mirror its intended operational environment but also guarantees its safety. The laboratory setting allows for a controlled environment where various parameters can be adjusted and studied in depth.

Standardized Tracking: The carbon tracking process adheres to globally recognized standards. This includes, but is not limited to, Covalent, Climate vault, Stripes, Puro, Vera Gold standard, and other notable standards. Adhering to these recognized standards ensures that the carbon tracking process is rigorous, accurate, and can be verified by third-party entities.

Ethical Standards: Transparency is paramount in this process. The operations abide by the code of ethics established by the AGU on mCDR research. This includes a “Killswitch mechanism,” ensuring that in the event of any unforeseen issues, the process can be halted immediately to prevent any potential harm.

In certain embodiments, the tracking of carbon loss during the seaway transportation process of the herein invention is further carried and established using a plurality of systems, which are not limited to the following. Gas Detection Systems monitor levels of various gases, including oxygen (O2), within the storage rooms, aiding in the detection of potential contamination or spoilage issues. Temperature Monitoring systems ensure that temperature levels within the cargo holds are maintained within specific ranges critical for preventing the growth of contaminants. Humidity Control systems are employed to regulate the humidity levels, further safeguarding the organic biomass during transit. Automated Sampling mechanisms are utilized to collect samples periodically from the storage compartments for detailed analysis, facilitating early identification of potential issues. CCTV Monitoring may be utilized indirectly to visually monitor the condition of the cargo, observing signs of spoilage or contamination. Integrated Management Systems compile data from various monitoring technologies to provide a comprehensive overview of cargo conditions, enabling proactive management and intervention. Remote Monitoring capabilities allow for real-time tracking of onboard conditions from a remote location, ensuring timely decision-making to mitigate any arising issues. Additionally, emerging technologies such as Bio-sensors may be explored for their potential to directly monitor the biomass for signs of contamination, although their implementation may still be in developmental stages. These systems collectively enhance the monitoring, verification, and reporting (MRV) capabilities of the transport process, ensuring the integrity of the carbon sequestration efforts as per the operational standards of the herein invention.

In certain embodiments of the MRV process of the herein invention, the determination of the depth of the anoxic zone is crucial for the efficient execution of carbon injection. This can be achieved through various methods to ensure precision and cost-effectiveness. In one embodiment, a preliminary environmental assessment is conducted prior to the injection operation, where sensors are deployed to measure the oxygen uptake at different depths to establish the anoxic zone's boundaries.

Alternatively, another embodiment involves the integration of sensors directly on the injection lance or needle described in the herein invention. This configuration allows for real-time data acquisition during the injection process, confirming the anoxic zone's depth as the operation progresses. The types of sensors used range from basic oxygen sensors to more advanced geophysical sensors that measure parameters such as sediment cohesive plasticity, shear stress, and compaction. These parameters include, but are not limited to, Young's modulus, Poisson's ratio, Biot's modulus, hydraulic conductivity, porosity, and pore fluid specific weight. The array of sensors employed provides a detailed understanding of the soil conditions, ensuring precise determination of the optimal injection depth and enhancing the effectiveness of the carbon storage.

Sensors utilized in this embodiment encompass a variety of instruments suitable to meet the specificities of the technological design used and injection area. Such instruments includes but does not limit to: oxygen sensors, which monitor oxygen levels to identify anoxic zones; pH meters that measure the soil's acidity or alkalinity; redox potential sensors for determining the oxidation-reduction potential; and conductivity meters to assess soil conductivity, which indicates mineral content and salinity. Additional sensors include soil moisture sensors, tensiometers, piezometers, nuclear density gauges, resistivity meters, seismographs, inclinometers, Time Domain Reflectometry (TDR) probes, capacitance probes, thermocouples, Resistance Temperature Detectors (RTDs), spectrometers, shear vane testers, and cone penetrometers.

This dynamic approach to establishing the anoxic zone via embedded sensor technology not only optimizes the injection process by ensuring a higher level of certainty in carbon storage and increasing revenue ($/tCO2 eq) of the herein design, but also enhances operational efficiency. It reduces the need for extensive preliminary evaluations and allows for real-time adjustments based on sensor readings. This method significantly reduces capital expenditures (CAPEX) and enhances economic viability.

In certain cases, the subsea metallic injection structure beneath the injection vessel, as previously described in the present invention, also integrates the sensors mentioned in the prior paragraph. In another embodiment, this metallic subsea injection structure is equipped with an echolocation scanner that enables scanning of the sediment layers at the site of injection before, during, or after the injection process. This capability allows for comprehensive verification of the sediment composition at the depth of injection and helps to identify any obstructions or debris that could impede the penetration of the injection lance into the seabed. Such obstacles could potentially damage the lance or result in an unsuccessful injection, thereby diminishing the effectiveness and efficiency of the herein invention.

Suitable seabed scanning technologies may include, but are not limited to, Acoustic Doppler Sonars, Multibeam Echo Sounders, Side-scan Sonars, Sub-bottom Profilers, Synthetic Aperture Sonars, Phase Measuring Bathymetric Sonars, Marine Seismic Reflection Systems, Seismic Refraction Equipment, Marine Magnetometers, Gradiometers, Conductivity Temperature and Depth Sensors, Acoustic Telemetry Systems, Laser Line Scanners, Photogrammetric Cameras, Lidar Systems, and Marine Gravimeters, as well as Acoustic Corers. These systems are adept at providing detailed images and profiles of the subsea environment, ensuring that the injection apparatus can operate at optimal locations and conditions.

Biomass degradation in the marine carbon dioxide removal (MCDR) system follows a first-order kinetic model, characterized by exponential decay. This implies that the highest rate of carbon degradation typically occurs during the initial phase post-injection. The monitoring, reporting, and verification (MRV) strategy implemented here hinges on the principle of functional stability, which asserts that the stability of the injected carbon can be accurately assessed after a continuous monitoring period of one year, in most cases, but up to 5 years and more in other cases. Pilot testing at the injection sites will involve intensive monitoring via advanced sensors specifically designed to detect any potential carbon leakage. These sensors help establish a threshold for functional stability, thereby providing a quantitative basis to evaluate the efficacy of the carbon sequestration process and ensure environmental compliance. This methodical approach to monitoring not only underscores the system's reliability but also enhances the predictability of long-term carbon storage outcomes.

In certain embodiments of this marine carbon dioxide removal (MCDR) and monitoring, reporting, and verification (MRV) system, advanced automation is intricately integrated into the injection process. This system incorporates numerous sensors directly installed on the corer injector needles, facilitating pre-penetration measurements within a critical time window before the needle enters the sediment layer. These sensors are tasked with scanning the seabed to evaluate the suitability of sediment depth for slurry injection, aiming to preemptively identify any anomalies such as unexpected geological formations or obstructions (whales or other) that could compromise the injection process. If a potentially problematic zone, such as a dense rock formation or an unusual sediment composition, is detected, the automation system can halt the operation, ensuring the safety and efficacy of the injection. This automated scanning process is swift, generally taking between 5 to 15 seconds, though it can extend up to five minutes for comprehensive analysis using technologies like radar, echolocation, and a suite of optical imaging methods. Methods include the deployment of various sensors that are not limited to the following: Radar sensors, Echolocation devices, Optical imaging cameras, Infrared cameras, UV light sensors, LIDAR (Light Detection and Ranging), Seismic sensors, and Acoustic Doppler current profilers (ADCP). These sensors are utilized to perform pre-penetration measurements.

Additionally, the integration of this automation extends into sensor capable of real-time monitoring prior injection phase and after the penetration of the injection lance within the sediment, with sensors installed on the injector lance. These sensors have the capability to provide the green light or stop the launch of the injection process. These high-speed sensors measure a wide array of environmental and chemical parameters including pH, dissolved inorganic carbon (DIC), dissolved organic carbon (DOC), temperature, redox potential, but does not limit to that specific list. These sensors would operate preferably within seconds to a few minutes. This rapid measurement capability is crucial for adapting the injection process dynamically, ensuring optimal conditions for effective carbon storage and minimal environmental disturbance. This automated sensor measurement process is swift, generally taking between 5 to 15 seconds, though it can extend up to five minutes for comprehensive analysis using technologies like radar, echolocation, and a suite of optical imaging methods.

In scenarios utilizing subsea metal structures for injection, as previously described, these sensors are mounted directly onto the structures themselves, enhancing the versatility and cost-effectiveness of the sensor systems. This strategic placement also facilitates broader measurement capabilities and easier integration with existing sensor technology markets.

The overarching goal of incorporating such advanced sensors and automation is to drastically reduce the labor involved in geotechnical monitoring, which currently accounts for a significant portion of operational staffing on the injection vessel described in the herein invention. By automating these processes, the labor costs and complexity of operations are significantly reduced, the overall time of injection operation is also reduced, enhancing the overall efficiency and reducing the cost of the carbon removal methodology.

In further embodiment, Automation is fully integrated into the current novel system of mCDR described here, encompassing every operational phase comprehensively.

This complete automation covers the deployment and management of injection needles, the dynamic positioning and navigational control of the injection platforms, and the deployment of sensors for monitoring throughout the pre-penetration, penetration, and post-penetration phases. Advanced geolocation systems equipped with GPS and gyroscopes enable the ships to execute precise injections autonomously, without the need for direct human oversight. Consequently, this system reduces the operational crew to a minimal monitoring team, significantly cutting down labor costs and minimizing human error. This embodiment ensures consistent and precise carbon sequestration across multiple sites, redefining efficiency and safety standards in marine environmental management.

The technology presented in this invention is a highly efficient system designed to capture and store atmospheric carbon for millions of years. It has the potential to make a significant impact on global carbon emissions and substantially reduce anthropogenic contributions. Depending on the scale of deployment, this technology could store between 0.5 GT and 3 GT of carbon annually, with the capacity to reach 3 to 10 GT in extreme cases and embodiment design. The primary objective of this system is to achieve a minimum of 0.5 GT of carbon storage per year-equivalent to a 1% reduction in global emissions—by 2050. To reach this target, the system is expected to scale up with a projected 10% annual growth rate starting from the year of the invention's publication.

To illustrate how this goal can be met, a standard embodiment of the system has been considered, along with a description of the minimum viable product (MVP) injector to help the reader understand the system's functionality and operation. The MVP is characterized by several key parameters essential to the system's efficiency, including the flow rate per injector, the total number of simultaneous clustered injections (SCI), and the slurry concentration. The inventors have defined the MVP with the following critical specifications: a flow rate of 400 L/min per injector, a slurry containment ratio of 1:50 (slurry to sediment space), the capacity to conduct 48 SCI at various depths, and a slurry concentration of 35% dry weight basis. These specifications represent the minimum thresholds required for the system to be viable on a large scale. It is important to note that as different standard embodiments described here can be considered, each key factor influencing the system's efficiency can adjust slightly.

### Exemplary Embodiments

Rail System for Continuous Injection: In a unique embodiment, the sea vessel could be equipped with a rail system running along the length of its hull. This system would allow the injection needle, winch, and other related components to be attached and move as the vessel travels. Instead of stopping and re-engaging at each injection site, the boat could maintain a constant speed ranging between 0.1-5 knots while the needles carry out their injections. The needle, sliding on the rail, would then be re-lifted and travel back to the front of the ship to reinitiate the injection operation, all while the ship remains in motion. This continuous movement reduces energy loss by eliminating the need to stop and restart the vessel, enhancing overall efficiency.

Onshore Slurry Preparation: In another embodiment, the slurry mixture might be prepared onshore, either at the port or directly at the biomass source. The slurry could be created using water or other compounds, such as oils, and then transported via adapted roadways as detailed in section 3.2, utilizing specialized transportation methods designed for slurry handling.

Onboard Biomass Generation: In further embodiments, the residual biomass could be directly generated on the ship while extracting compounds from the biomass via the green extraction processes described in Choquet et al, patent US 2023/0132519 A1. This direct generation would streamline the process, integrating the extraction and preparation of biomass.

Dry Biomass Injection Methods: In some embodiments, the injection of organic biomass might be performed without using liquid pumps. Instead, dry biomass could be deployed using mechanical systems such as a piston corer, leveraged by a weight leveler in the sea. Other methods might include systems similar to pylon installation in channels for aquaculture or signs, or utilizing hydraulic jacking systems commonly used in geotechnical applications to inject dry biomass into mud.

Submersible Mixer for Slurry Preparation: In other embodiments, the slurry might be mixed directly in the water column using a submersible mixer, ranging at depths of 1 to 4000 meters. This submersible mixer could be attached to the same corer needle winch or an additional winch at a distance of 2 to 30 meters alongside the needle's vertical setting in the water column.

Subsea pump Integration: In similar scenarios, subsea pumps could be used for the injection process, integrating them into the system in a manner that complements the other components.

Airtight Mixer Chamber for Gravity Injection: In a novel approach, the slurry might be allowed to fall through gravity down a large flexible pipe attached to an airtight mixer chamber. Equipped with valves, this chamber would control the water and slurry flow, utilizing pressures ranging from 50-500 bars. Gases like CO₂ could be injected to push the water out, enabling repeated injection operations.

Slow-Paced Injection with Rail System: In yet another embodiment, the ship might maintain a slow pace, ranging from 0.1 to 3 knots per hour, during injection. The needles would be attached to winches placed on a rail, allowing for precise, controlled injection while the vessel remains in motion.

Subsea Injection Pumps at Various Depths: In different embodiments, subsea injection pumps might be utilized at depths of 30-200 meters above the seabed, attached either to the corer needle cable or an additional vertical cable at a distance of 2-50 meters. This configuration offers flexibility in the injection process.

Multi-Pump System for Friction Reduction: Finally, in a comprehensive approach, a multi-pump system might be spread through the overall lines, including one on the boat, one in the subsea, and one at the needle for injection. This system allows for a reduction in friction and a more efficient operation, with the potential to integrate various other embodiments as well.

Adapted Weight Hammer Systems: As described in section 3.3, the adapted weight hammer systems, similar to sediment coring, are actioned at TO of the injection to counterbalance the popping-out effect of the needle due to the injection pressure in the borehole. This system could additionally reduce the need for the weight stabilizer of the needle and decrease the emissions linked to the lifting up of the corer needle by the corer needle winch, vertical line. This innovative approach enhances the stability of the needle during the critical injection phase, contributing to the precision and efficacy of the process.

Unfoldable Grip on the Needle: The needle could feature 2 to 4 unfoldable grips as large as the surface of the needle's circumference. These unfoldable grips would be deployed once the needle is buried in the sediment and would open facing the surface. They are designed to provide additional grip to the needle corer during injection, to avoid the effect of the needle popping out. These would have the ability to reduce the need for a large needle stabilizer weight and reduce the emission linked to the winch usage to lift this weight up. Once the injection is over, the corer needle hammer weight would be activated to further bury the needle a few meters, allowing the foldable grips to refold. These grips, being part of the needle's outer structure (metal), would not change its hydrodynamic shape.

Corer Needle Adaptation with Drill: In another embodiment, the corer needle is an adaptation of a drill needle. An electric motor or system is included directly on the needle structure, allowing the rotation at a high speed of the rotative screw head of the corer needle.

This facilitates the penetration of the corer needle to the appropriate depth, adding a layer of flexibility and adaptability to the injection process.

Needle with Outside Spacing for Liquid Flow: In another embodiment, the needle contains outside spacing that would allow liquid flow through an exit tube. This enables the exit of excess water injected in the slurry mixture, reducing the need for water diffusion through the borehole and through the needle to reduce pressure, thereby gaining efficiency. This precise control over the flow dynamics within the needle enhances the overall performance of the system.

Principal Embodiment of the Flexible Pipe System: In the principal embodiment, the flexible pipe system that allows the biomass to travel from the ship down to near the seafloor is carried through the use of a flexible tube that adapts to the hydrostatic pressure. This tube allows less energy to be needed by the displacement pump that is placed on the ship. In this embodiment, more water is used with the biomass to reduce the compressibility of the slurry and reduce the need for the pump to fight the hydrostatic pressure of the bottom water. The biomass can slowly compress during its descent. This tube starts with a bigger diameter and finishes with a smaller one, correlated with the current concentration of the biomass and its initial volume in the pipe. This primary embodiment for the flexible pipe allows a much lower pressure capacity pumping of the displacement pump and immense reduction of energy to provide a bottom subsea pump with a slurry already at local pressure. This significantly reduces the pressure need for the subsequent injection pump to fracture the sediment, offering a more energy-efficient approach to the entire process.

Bulk Biomass Transport via Conveyor Piping with Hydrostatic Pressure Adaptation: In another embodiment, the bulk biomass is transported from the surface water column to the bottom via the use of conveyor piping containing bowls and rods that force the biomass to descend. The pipe system in this configuration contains holes, allowing for the adaptation to hydrostatic pressure. This ensures that the pressure within the pipe system is balanced with the surrounding water pressure, reducing stress on the structure. A cable rotator system continuously transports biomass in a closed-loop system, further enhancing the efficiency of the transportation process. This unique design offers a combination of continuous flow and pressure adaptation, providing a robust solution for biomass transport.

Spiral Piping for Biomass Transportation: In another embodiment, the biomass is transported downward through the use of spiral piping, once again forcing the biomass downward. The pipe system in this configuration also includes openings or holes to allow for the adaptation to hydrostatic pressure. This ensures the internal pressure is balanced with the surrounding water pressure, minimizing the risk of rupture or deformation. The spiral design creates a controlled downward flow, propelling the biomass to the desired depth while utilizing the surrounding water pressure to maintain a stable flow rate. This combination of spiral movement and pressure adaptation forms an energy-efficient solution to transport biomass to the deep-sea floor.

Biomass Transport via Permeable Flexible Tubing: In another embodiment, the biomass is transported downward through the use of a permeable flexible tubing onto which several flow pumps are placed (every 50 to 100 meters) to create a downward flow current. This allows the transport of the biomass to the bottom of the water column, utilizing a combination of permeability and controlled pumping to maintain a precise flow.

Biomass Concentration Adjustment Chambers: In the three previous embodiments, the biomass reaching the bottom of the water column, already under pressure, would enter small chambers that readjust the biomass-to-liquid concentration to a range of 25% to 90%, with preferable range between 30% to 55%. This is then fed to subsea pumps to carry out the injection fracture of the slurry with low pressure displacement. This design greatly reduces the overall energy and emission requirements, enhancing the Emission to Removal ratio of the invention.

Multi-Line Needle Packages on Ships: In another embodiment, ships contain several lines and needle-line packages, allowing them to carry simultaneous injections to maximize both Opex and Capex efficiency. The distance between each needle package can vary from 30 to 100 meters and can be on each side of the boat, depending on the vessel's beam size. For example, a capsize (180,000 TEU size) bulk ship carrier with a 50-meter beam length and 200-meter hull length size will carry ten pumps and needle corer packages, each spaced 50 meters apart. This design is adaptable to various bulk ship sizes, offering scalability and adaptability.

Telescopic Reels and Rail Systems: In other embodiments, telescopic reels are installed on the ship, extending further away from the distance of its hull length, allowing more corer needle packages to operate simultaneously. Additional rails placed perpendicularly to the direction of the hull are mounted on the ship, further increasing its capacity for simultaneous injection. This configuration provides increased flexibility and the capacity to handle multiple injections at once.

Increased Simultaneous Injection Capacity: In the two last embodiments, the total amount of injections that can be carried simultaneously can vary up to 50, allowing much faster delivery time and operations at sea. This reduces the Opex of the crew, the injection distance required by the ship, and the Capex linked to the ship. More biomass can be efficiently stored and transported, maximizing the overall efficiency and sustainability of the process. These embodiments reflect a comprehensive approach to scaling and optimizing the system, ensuring its adaptability to different operational needs and environmental conditions.

Mobile Platform for Sea Operation: In another embodiment, the operations are not conducted by conventional ships but by a specialized platform optimized for sea operation. This platform is designed to move from one injection point to another and is constantly resupplied with the necessary fuel for energy power and biomass via conventional bulk carrier ships (commissioned or not). Such a platform could also benefit from telescopic rails that could accommodate several needle packages, allowing for increased flexibility and efficiency in the injection process. This design would enable the platform to adapt to varying operational requirements and environmental conditions, enhancing the overall effectiveness of the carbon sequestration method.

Integration with Algae Bloom Recovery: In another embodiment, for operations that are geographically near zones facing heavy algae blooms, the system integrates the support of additional specialized ships. These ships are tasked with collecting seaweed and algae from blooms, so this biomass can be transported back to the main vessel for direct injection. The support of specialized ships enables the direct recovery of seaweed and algae blooms, turning an environmental challenge into a valuable resource. By partnering with these specialized ships, the system efficiently transforms the recovered algae into biomass suitable for carbon sequestration, further contributing to the innovative approach's adaptability and environmental sustainability.

Near Shore Operation (5 to 1000 miles from the coast): In the case of nearshore operations, where the depths are less than 900 meters, this technology remains applicable and even offers certain advantages. The proximity to shore allows for easier access to more efficient energy sources, such as electricity, further enhancing the system's effectiveness and sustainability. This flexibility in operation depth and location demonstrates the invention's adaptability and potential to contribute to carbon sequestration in various marine environments. In this case it's possible that the risks of carbon leakage are increased yet well more scientific evidence will be required. It is hypothetical that the system would still be somehow viable due to the low amount of decomposition of the biomass via respiration of sediment microbes.

In another embodiment, additional integration of other Carbon Dioxide Removal (CDR) approaches can be incorporated to enhance the system's efficiency and reduce emissions further. For example, collaboration with technologies like those developed by SeaBounce, an English company, can be considered. SeaBounce manages to recover the CO₂ CO₂ emissions from the seavessel's exhaust system and convert the CO2CO₂ into liquid form, suitable for injection in land-based wells. Such a system would significantly diminish the emissions associated with the consumption of diesel fuel, a requirement for almost every aspect of this approach. While this integration may marginally increase the capital expenditure (CAPEX), it would notably decrease the emissions linked to the project. Despite the high efficiency of the project enabling it to reach a minimal cost per ton of CO2CO₂, the process can afford to invest more in the management of its fossil fuel emissions. By implementing this system, the Emission to Removal Ratio (ERR) would be greatly improved, for instance, from ERR=0.072 to values ERR>0.01. This embodiment not only emphasizes environmental responsibility but also ensures the project's alignment with sustainability goals and regulations.

In a distinct embodiment, the invention introduces a circular multi-micro injection system that represents an innovative approach to biomass injection. In this configuration, the head of the pump is designed as a disk containing several thin injection needles, ranging from 2 to 5 meters in length, and housing anywhere from 2 to 30 small injection needles. The system is engineered to inject biomass simultaneously through each of these smaller needles, optimizing the process for efficiency and uniformity. Injections can be carried out at depths ranging from 0.5 to 10 meters, with varying spacing between each needle, offering flexibility in adapting to different geological conditions and biomass characteristics. In other variations of this embodiment, the structure containing the needles may deviate from a disk to assume other geometrical forms, further enhancing the system's adaptability and effectiveness. This embodiment embodies the inventive spirit of the methodology, utilizing precision engineering to achieve precise, simultaneous injections, thereby maximizing the efficacy of the carbon sequestration process.

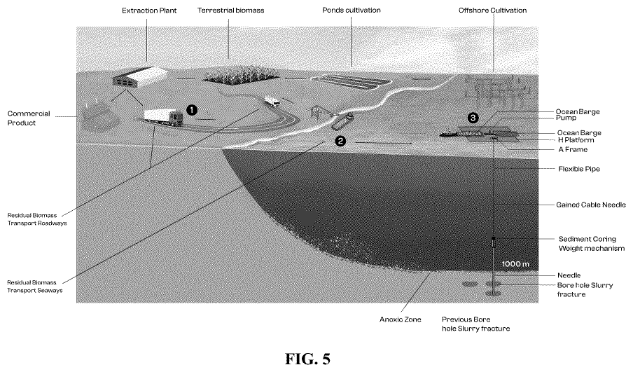

The mobile injection platform configuration as delineated in paragraph [V1 199] is further elucidated to encompass an ‘H’-shaped design, previously conceptualized for specific maritime applications or part of a novel injection platform architectural design (see FIG. 5). This configuration comprises injector needles strategically positioned on the lateral sides—the external sides of the ‘H’. Such an arrangement may include, but is not limited to, six injectors per side, culminating in a total of twelve injectors. The spacing between these injectors may range generally from 30 to 50 meters more or less, adhering to the parameters established in prior disclosures, and define the width of the here described design of injection platform.

The distinctive ‘H’ shape of the injection platform facilitates logistical operations by enabling ocean barges to dock within the central recess of the ‘H’. This design supports efficient delivery processes where a trawler vessel tows an ocean barge loaded with biomass to the platform, docks it, detaches from the ocean barge, and subsequently retrieves the emptied barge from the other docking point to return to port. The swap operation of the trawler vessel can span from as brief as five minutes to an hour, with an optimal timeframe averaging around 20 minutes.

In such embodiments, the dimensions of the ‘H’ platform are tailored to match the standard dimensions of ocean barges. The platform can extend from 170 to 200 meters or more in length, accommodating two large ocean barges with a capacity of 5000 DWT each (approximately 72 meters in length), aligning with industry standards for maritime operations. In alternative designs, the injection platform may present a slightly different configuration in terms of length and width and will accommodate ocean barges of varying capacities, ranging from 200 DWT to 20,000 DWT. In some designs, the platform is significantly larger and capable of accommodating entire transport vessel of various size (i.e. sea bulk carrier).

Depending on the injection capacity of the platform, two to three or more ocean barges are deployed to maintain a continuous supply, thus facilitating uninterrupted injection operations. The platform is equipped with dynamic positioning capabilities and other similar technologies, as previously described. Notably, the biomass stored on the ocean barges docked at the platform is directly funneled into the injection system, eliminating the need for afloat transfers. This biomass is efficiently supplied to mixers via mechanical converters, suction, or other standard methods currently used in the industry. These feed multiple pumps located on the platform, with each pump serving three to four injector needles, thereby supporting six to twelve or more injectors in total, consistent with the design specifications outlined earlier.

In this embodiment, the ability to dock ocean barges directly at the injection platform, coupled with the use of dynamic positioning, significantly enhances the platform's overall injection capacity by eliminating the time required to offload biomass from the barges prior to injection operation. The dynamic positioning system enables precise maneuvering of both the platform and any docked barges during injection operations. This integrated approach ensures efficient biomass handling and optimizes the annual injection operational capacity throughput of the platform.

While the ‘H’-shaped design of the injection platform is currently the preferred method to the large scale application of the herein technology, it is not the sole configuration compatible with this invention. Alternative platform geometries offer equal viability and can be customized to meet the specific operational requirements. In a refined variant of the ‘H’ design, enhancements include retractable mechanisms aimed at securing ocean barges firmly to the platform, thereby ensuring stability and safety during operations, particularly in challenging sea conditions. This feature is especially crucial in mitigating potential damage to barges equipped with more rudimentary securing methods, such as ropes. The current design significantly enhances operational efficiency by extending the platform's capability to operate in rough seas, consequently increasing the annual injection time per year.

In regard to the injector needle, other embodiment further includes needles with length, ranging from 5 to 25 meters or from 25 to 50 meters, 50 to 900 m in certain embodiment using more sophisticated method of coring previously described. These injection lances employ Vibro-Coring methods or other penetration techniques described in the present invention to traverse through the sediment. The total number of smaller injection needles can vary from 1 to 30 and 30 to 60 per injection vessel and more in certain design.

### Exemplary Subsea Submersible Slurry Pump Design

In certain embodiments alloys like Hastelloy C-276 or Inconel 625 for the pump's construction material is crucial. The alloys are specifically chosen for their high resistance to localized and stress corrosion, which are common forms of degradation in underwater and high-pressure environments. Ensuring that these materials undergo ASTM G48 testing provides additional assurance that they can withstand pitting and crevice corrosion, both of which can compromise the integrity of the pump.

The pump is designed to operate within a specific pressure range of about 70-bar to about 125 bar, preferably about 95 to about 105 bars. This range provides a safety margin above the typical 70-bar requirement for sediment fracturing in subsea operations. It allows for operational flexibility and minimizes the risk of pump failure due to unexpected pressure spikes, making the system more reliable for long-term use.

An alternative design uses Titanium Grade 5 for the pump's structural elements. This material is chosen for its strong tensile strength and exceptional corrosion resistance. Its properties make it ideal for deep-sea operations, extending the pump's durability and reliability. Additionally, the modular design of the pump allows for various attachments, making it adaptable for transporting different types of slurries and accommodating different particle sizes.

The electric motor is preferred to have a power rating of about 65+5 KW. This ensures that the pump has ample operational capabilities while optimizing energy consumption. The motor housing is designed to conform to an IP68 rating, which guarantees that it remains watertight. This is especially important for underwater applications where the integrity of electrical components is crucial. Moreover, the pump's ability to handle voltage fluctuations within +10% enhances its robustness and adaptability to different power supply conditions. In exemplary scenarios where electrical power is not feasible or desirable, a hydraulic motor can be employed as an alternative. This motor variant uses hydraulic fluid like ISO VG 46, channeled from a surface vessel via armor-reinforced hydraulic lines.

These lines are designed to withstand pressures up to 200 bar, making them resilient against potential pressure spikes. Additionally, the hydraulic motor includes a thermal overload sensor to monitor the hydraulic fluid's temperature, ensuring it stays within the optimal range for high performance. To ensure the seamless transmission of rotational force from the motor to the pump, a motor coupling made of marine-grade 316 stainless steel is employed. This coupling incorporates splines or keys that align perfectly with the pump shaft, ensuring efficient power transmission. It undergoes cyclic load testing to ascertain its ability to handle a specific range of rotational torque, significantly reducing the risk of mechanical failure during operation.

For applications requiring additional safety measures, an advanced magnetically coupled design is available. This design incorporates a built-in torque limiter that acts as a mechanical “fuse.” If the torque exceeds a certain limit, the limiter disengages, thereby protecting the system from potential overloads or jamming events. This feature adds an extra layer of security to the pump system, safeguarding both the equipment and the operation.

In an exemplary embodiment, the thrust bearing and shaft are made from a proprietary blend of Type 416 stainless steel and molybdenum. This specific material selection ensures a balance between high tensile strength and corrosion resistance, attributes that are essential for underwater operations. The components are subjected to stress tests to confirm their axial load capacity, which is specified to be up to 1000+50 N. Moreover, the shaft has a diameter of 3+0.1 inches to maintain optimal hydraulic efficiency, reducing the risk of performance loss over time. In an alternative design, ceramic-based bearings made of zirconium dioxide (ZrO2) can be used. These bearings offer superior load capacities-up to 1500+50 N—and have lower friction coefficients. This results in less wear and tear on the bearings, thereby extending the overall lifespan of the pump system. The impeller is designed from high-quality marine-grade bronze and is engineered to deliver specific flow rates ranging between 550 and 650 liters per minute. The blade design is hydrodynamically optimized to maximize fluid flow while minimizing energy consumption.

Computational Fluid Dynamics (CFD) tests validate the efficiency of the blade design. The clearances between the impeller and the casing are maintained at about 0.5+0.05 mm, which helps to minimize hydraulic losses that could otherwise reduce the pump's performance. An alternate embodiment for the impeller uses a composite material made from carbon fiber and epoxy resin. This alternative offers adjustable impeller blades that is preferably fine-tuned in real-time to optimize the pump's hydraulic characteristics. This adaptability allows for a wider range of flow rates, from 400 to 700 liters per minute, offering greater flexibility in various operational scenarios.

The pump casing is constructed from ductile cast iron, ensuring robustness and durability. It has a minimum wall thickness of 10 mm and is coated with epoxy for added protection against corrosion. The design incorporates a perforated inlet screen with 2-mm diameter holes, serving as a pre-filter to prevent large debris from entering and potentially damaging the pump system. For more demanding applications, an alternative double-walled casing made from duplex stainless steel, conforming to ASTM A890 Grade 4A, can be employed. This advanced design includes a 1-mm vacuum layer between the inner and outer walls, providing extra insulation and structural integrity. This additional layer enhances the casing's resistance to extreme pressures and temperatures, making it a robust option for more challenging environments. To ensure the seamless transmission of rotational force from the motor to the pump, a motor coupling is preferably made of marine-grade 316 stainless steel. This coupling incorporates splines or keys that enable perfect alignment with the pump shaft. To validate its robustness, the coupling undergoes cyclic load testing, ensuring it can tolerate rotational torques of up to 500+25 Nm.

An alternate design features an advanced magnetically coupled system with a built-in torque limiter. This safety feature automatically disengages at torques of 450+10 Nm, acting as a mechanical fuse to protect the system from overloads or jamming events. This adds an extra layer of safety and ensures the longevity of the pump system. The check valves are made from Monel, a nickel-copper alloy renowned for its superior corrosion resistance. These valves are spring-assisted, which ensures rapid closure and minimizes the risk of backflow. They are rated to operate within a pressure range of 65-75 bar and are strategically positioned at intervals of 5+0.1 meters along the drop pipe. As an advanced alternative, “Smart” check valves equipped with MEMS (Micro-Electro-Mechanical Systems) sensors are available. These smart valves not only control the flow but also measure critical operational parameters like pressure, temperature, and flow rates. This data is preferably relayed to the control system for real-time adjustments, enhancing the pump's overall performance and reliability.

The electrical cabling is preferably designed to meet the power demands of the system. It features a double-insulated jacket made from cross-linked polyethylene (XLPE), which is rated to handle voltages up to 1000 volts. To further enhance its durability, the cables are armored with galvanized steel wire braids, capable of withstanding tensile stresses up to 2000 N.

In an alternative design, fiber optic cables can be integrated into the electrical cabling. This allows for rapid data transmission between the pump and the control system, enabling real-time monitoring and adjustments. This high-speed data capability is particularly beneficial for optimizing performance and troubleshooting issues as they arise.

The control system of the pump is preferably anchored on an industrial-grade Programmable Logic Controller (PLC). To withstand the harsh environmental conditions, the PLC is housed in a pressure-resistant enclosure made from 6Al-4V titanium alloy. For added reliability, the system features redundant sensors that continuously monitor operational parameters such as pressure, temperature, and flow rates, thereby ensuring optimal performance.

An alternate embodiment of the control system offers cloud connectivity, allowing for remote monitoring and operational adjustments. This advanced version integrates machine learning algorithms, enabling the system to optimize its efficiency dynamically and predict maintenance needs or potential system failures. This represents a leap in the pump's adaptability and operational intelligence. The pump features a variable speed drive, allowing it to operate over a broad speed range between 1000 and 3000 RPM. This adaptability enables the pump to rapidly adjust its speed to maintain an optimal flow rate between 550 and 650 liters per minute, catering to varying operational needs.

In an alternative design, a dual-speed drive system can replace the variable speed drive. This system would offer a high-speed setting of 2800 RPM and a low-speed setting of 1500 RPM, providing more straightforward speed control options while still catering to different operational requirements. The pump is engineered to preferably handle batch operations, with the capacity to pump a total slurry volume of 20+0.2 cubic meters per batch. It also features a built-in buffer capacity of 2 cubic meters to accommodate any slurry expansion that may occur due to temperature or pressure fluctuations.

In alternative embodiments, the pump is preferably scaled up, including key components like the motor shaft and other power-related parts. With these modifications, the pump's slurry batch capacity can range from 40 to 500 cubic meters per batch. This allows for greater flexibility in handling larger volumes of slurry, thereby increasing the system's overall efficiency and throughput.

As an alternative, a dual-chamber system could be incorporated, allowing for continuous operation by filling one chamber while the other is in the pumping phase. This enhances the system's throughput and operational flexibility, making it adaptable to a range of industrial applications. The pump is preferably designed with a self-priming mechanism, which negates the need for any external priming systems. This attribute is particularly advantageous as it enables the pump to initiate operations promptly and efficiently upon start-up.

In an alternative design, the self-priming system could incorporate a built-in recirculation line. This feature would use a small fraction of the pumped slurry to prime the pump, further enhancing its reliability and operational readiness. Safety is a paramount consideration in the pump's design. The control system is imbued with several safety mechanisms, including overcurrent, overpressure, and thermal overload protection. These features are programmed to trigger emergency shutdown procedures, thereby minimizing equipment damage and averting potential safety hazards. Compliance with relevant ISO and ASTM safety and reliability standards is assured in the design.

The pump system is preferably engineered to facilitate easy deployment and retrieval. It features multiple attachment points compatible with standard ROV (Remotely Operated Vehicles) tooling. This design choice simplifies both the installation and recovery processes, making it user-friendly for operators in various marine environments. The pump system comes equipped with an onboard data logging system, boasting a storage capacity of 64 GB. This feature is invaluable for capturing real-time operational data, such as pressure, temperature, and flow rates. In an optional configuration, this logged data can be transmitted in real-time to a surface vessel or an offshore facility using a high-speed data link. This real-time data transmission allows for immediate analysis and decision-making, enhancing both the efficiency and safety of the pump's operation.

The simultaneous multiple injections mentioned in paragraphs [V1 171], [V1 196], [V1 197], and [V1 198] of this invention include: a) horizontal injection, facilitated through side-by-side placement of injector needles as previously described; b) vertical injection, achieved through stratified injection along the needle shaft; and c) a combination of both horizontal and vertical injections performed simultaneously. This configuration enhances the flexibility and effectiveness of the injection process by ensuring precise placement and distribution of the injected material. For all simultaneous injection methods—a), b), and c)—all system embodiments described in this invention are applicable and suitable. Additionally, within the context of this invention, the term “multiple injection” and any similar terms refer specifically to any of the embodiments a), b), and c) detailed in this paragraph.

For vertical simultaneous injection, the distance between each injection point typically ranges from 0.1 m to 3 m and 3 m to 20 m. However, in certain scenarios, these distances can extend further, ranging from 20 m to 50 m, and from 50 m to 100 m, up to 100 m to 900 m in extreme cases.

In the case of simultaneous horizontal and vertical injection, as previously established in paragraph [V1 198], the total number of simultaneous injections can range from 50 to 500. In certain embodiments, the total number of injections can vary from 1 to 100, with slurry quantities per injection point ranging from 100 to 200 m³, 200 to 5,000 m³, and 5,000 to 30,000 m³. For slurry quantities of up to 100 m³ per injection point, the range of simultaneous injections spans from 100 to 500.

In certain embodiments, the total quantity of multiple simultaneous injections ranges from 1 to 12, 12 to 72, 72 to 200, and 200 to 500, depending on the specific design.

In the context of the present invention, the operational methodology involving multiple injection needles operating in a coordinated manner-either vertically or horizontally aligned within the sediment—is central to enhancing the efficiency and precision of slurry distribution into the sediment. This methodology, where multiple needles are synchronized to inject simultaneously, is a core aspect of the performance of the system described in this invention.

This concept, fundamental to the herein described technology, may be referred to by several terms, reflecting its various aspects: Clustered Multi-Injection Synchronization (CMIS), Concurrent Clustered Injection (CCI), Clustered Multi-Injection (CMI), Simultaneous Clustered Injection (SCI), Synchronized Multi-Needle Injection (SMI). Additional terms that also encapsulate this concept include but are not restricted to: Coordinated Injection Array (CIA), Integrated Needle Synchronization (INS), Parallel Injection Synchronization (PIS), Unified Multi-Needle Deployment (UMD). These terms underline the strategic arrangement and operational synchronization of injection needles, critical for achieving the desired outcomes in sub-sediment carbon sequestration as detailed in previous sections of this document.

The term “injection points” refers to the specific locations where slurry is introduced into the sediment. These points may be aligned horizontally at a consistent depth within the sediment or arranged vertically with multiple points stacked one above another. More precisely, the term “injection point” can refer to either a set of apertures grouped at the same vertical depth on the needle shaft or to each individual fracture generated from each aperture within the vertical set on the needle.

As the invention continues to develop, a new embodiment has been conceptualized to enhance the stability and geo-precision of the injection operation. This new embodiment involves the use of a vertically mobile metallic platform, specifically designed to fit under the injection vessel and capable of traveling from the surface down to the near seabed. In this embodiment, the metallic platform is constructed in a long, squared or rectangular shape, tailored to the dimensions of the injection vessel, whether it be a ship or platform. The design allows this subsea metallic injector structure to be retracted under the vessel for long-distance travel (ie commute to other biomass hub, sheltering during storms). The platform is equipped with a vertical mobility system, which enables the subsea metallic injector structure to be lowered from the surface to the seabed. Mounted on this platform are multiple injector needles, which can operate using one or a combination of the previously described injection systems. These include:

1. Modified Gravity Corer Injector Needles: Similar to the previously described system, these injector needles can detach and drop from the metallic structure, utilizing gravity to penetrate the seabed.

2. Static Injector Needles: Static Injector Needles: In another embodiment, the injector needles are static, with lengths ranging from 1 to 15 meters, 15 to 30 meters, and 30 to 80 meters. The subsea metallic injector structure is designed to be gradually lowered onto the seabed. The substantial weight of this metal structure aids in the penetration of the injector needle into the soft seabed sediment. In certain designs, the static needle incorporates a vibrating tool to facilitate penetration into denser sediment. This new embodiment offers significant advantages: The subsea metallic injector structure ensures precise and stable seabed penetration, minimizes the risk of overlapping injection areas, reduces the time between each injection operation, and enhances sequestration efficiency. Furthermore, more accurate spacing between injections reduces the required seabed area for effective carbon storage.

An additional embodiment of the injection vessel is described in the following section. This injection vessel, referred to as the spud barge-modified injection platform, is designed for rapid deployment and retraction of injection mechanisms within shallow coastal environments, particularly on continental shelves with depths ranging from 5 to 100 meters. This platform incorporates several key components typically found in standard spud barges, which are modified to suit the unique requirements of the carbon sequestration process described herein. Notably, in this design, the spud legs themselves function as Sinkcore injectors, varying in number from 3 to 10, 10 to 50, 50 to 100, and up to 250 legs in extreme cases and specific design embodiments.

The spud legs are essential for the platform's stability and penetration into the seabed, functioning as Sinkcore injectors. These spud legs are configured in various forms, including fixed-length, telescopic, vibratory, or hydraulic designs, and also feature, in certain embodiments, spud cans, helical designs, lateral support struts, or impact hammers to enhance their functionality. Given their role as both stabilizers and injectors, these legs are modified to include additional features such as penetration sensors or reduced diameters to accommodate the increased number of legs required for precise and stable operations. The beam of the platform, or the barge itself, varies significantly depending on the specific embodiment. In certain designs, the barge length may range from 20 to 50 meters, 50 to 75 meters, 75 to 150 meters, and up to 150 to 300 meters, with beam sizes ranging from 10 to 30 meters, 30 to 50 meters, and in extreme cases, from 50 to 75 meters or even 100 meters. These dimensions ensure the platform provides the necessary support and stability for the injection operations while accommodating the extensive number of spud legs.

The jacking system is another critical component of the Sinkcore injector spud barge vessel, responsible for elevating and retracting the platform. This system includes hydraulic, electric, or rack-and-pinion mechanisms, with features such as high-speed, load-compensated, or auto-leveling configurations but is not restricted to them. This jacking system requires reinforcement or slight modifications to manage the additional spud legs, ensuring robust and efficient operation. The system's speed varies, with retraction and deployment times ranging from 1 to 5 minutes for rapid repositioning and extending to 10 to 15 minutes for specialized configurations.

The hull structure of the platform is constructed from steel, composite materials, or reinforced concrete, designed to support the platform's weight and ensure stability. The hull features corrosion-resistant coatings, double-bottom designs, or modular configurations. Given the operational demands of the injection process, these standard hull structures are adapted to provide additional rigidity and stability, ensuring the platform can safely bear the weight of the numerous spud legs and associated injection equipment. in alternative embodiment, the hull is designed using lightweight aluminum or ice-reinforced materials, depending on the environmental requirements.

The mobility system of the spud barge is a key consideration, enabling the platform to reposition itself between injection sites. This system includes hydraulic spud retraction, thruster-assisted propulsion, dynamic positioning systems, and GPS-guided movement but is not restricted to these configurations. For the modified platform, these systems require enhancements, such as increased spud deployment and retraction speed or improved precision in movement control, to ensure the platform can quickly and accurately reposition during operations. The movement range of the platform from a injection site to another is directly related to the configuration of the horizontal injection design, with the ability to make precise adjustments using thrusters or dynamic positioning systems.

These components, though similar to those found in traditional spud barges, are specifically modified to accommodate the unique demands of the carbon sequestration process, ensuring the platform operates effectively in various environmental conditions. It is important to note that these designs are not restricted to the specific configurations mentioned here, as further modifications may be necessary to optimize performance for different operational scenarios. The invention encompasses a broad range of design variations, ensuring adaptability and flexibility in the deployment of the spud barge-modified injection platform.

An additional embodiment of the Sinkcore spud barge injector vessel is a jack-up lift Sinkcore injector platform, which is more suited for farther offshore operations with greater water column depth. This platform is specifically designed to operate in deeper waters and withstand rougher seas.

The jack-up lift platform functions similarly to the spud barge but offers enhanced stability and operational capacity in more challenging marine environments. Equipped with larger and more robust spud legs, this platform can elevate itself several meters above the water level, providing a stable base for injection operations even in deeper waters. The platform is capable of handling larger quantities of biomass and conducting fewer simultaneous injections compared to the spud barge, but with the advantage of injecting larger volumes at each point.

### Examples

### Early Performance Analysis of the System:

Based on a preliminary LCA conducted in July and August 2023, here are the foundational assumptions for our analysis:

| Assumption | | | |
|---|---|---|---|
| Category | Details | Value | Unit |
| Scale | Technology Readiness | Commercial Scale TRL10 | — |
| Biomass | Type | Terrestrial crops residue | — |
| Density of Slurry | | 1,190 | kg/m³ |
| Moisture Content | Exclusion | Not accounted for | 10% |
| | | during transport | |
| Ship Capacity | | 15,000 | tones |
| Number of Trips | | 37 | — |
| per Year | | | |
| Emissions | Biomass Production | None (considering | — |
| | | wood waste) | |
| Emissions | Biomass Transport | included | — |
| Pump Power | | 1,087.5 | kWh |
| Pump Pressure | | 300 (Conservative) | Bar |
| Combustion | Diesel Engine | 40 (Realistic target) | % |
| Efficiency | | | |
| Transport Distance | Organic Biomass to Port | 300 | km |
| Transport Distance | Organic Biomass to injection | 243 | Nautical |
| Ship | location | | miles |
| Capex | Bulk Carrier Handysize | $33,264,000 | $ |
| | Needle Corer + Hydrostatic | $1,188,000 | $ |
| | Resistant Tube | | |
| | High-Pressure Hydraulic Pump | $297,000 | $ |
| Economic Project | | 30 | years |
| Life | | | |
| Annual Capex | | $1,203,506.21 | $/year |

### Results Analysis Example 1

| | | Total CO₂ | | |
|---|---|---|---|---|
| | | Emissions | | |
| | | (kg per | Percentage | Emission |
| | Total CO₂ | Gross | of Total | to |
| Production | Emission | ton of CO₂ | CO₂ | Removal |
| Steps | (Kg/year) | stored) | Emission | Ratio |
| Raw Material | 0 | 0 | 0% | 0.000 |
| (Capture) | | | | |
| OPEX Transport | 12,124,036 | 15 | 20% | 0.015 |
| OPEX Injection | 49,984,357 | 62 | 80% | 0.062 |
| Total CO₂ eq. | 62,108,393 | 77 | 100% | 0.077 |
| Emissions | | | | |
| Gross CO₂ Stored | −802,790,437 | | | |
| Net CO₂ Balance | −740,682,044 | | | |

| | Total Cost | Cost CDR | Percentage of |
|---|---|---|---|
| Production Steps | ($/year) | ($/ton of CO₂) | Total Cost |
| Raw Material (Capture) | 14,060,973 | 19 | 34% |
| OPEX Transport | 5,686,773 | 8 | 14% |
| OPEX Injection | 18,553,757 | 25 | 45% |
| Capex Injection | 1,203,506 | 2 | 3% |
| Salary Team | 1,672,749 | 2 | 4% |
| Total Cost | 41,177,759 | 56 | 100% |

Analysis: From the results, it's evident that the cost associated with this mCDR approach stands at $56 per tCO₂, presenting an emission to removal ratio (ERR) of 0.077. Delving deeper:

Emissions Analysis: The majority of emissions, 80%, come from the OPEX Injection phase, making it the most emission-intensive segment. In contrast, raw material capture contributes virtually zero emissions, showcasing the environmental friendliness of using waste materials.

Cost Analysis: The largest cost contributor is the OPEX Injection, accounting for 45% of the total. This is followed by raw material capture at 34%, suggesting that while the capture process is environmentally friendly, it remains a significant cost driver. Transport costs and Capex Injection are relatively lower contributors due to the short distance.

Potential for Optimization: Given the high emissions and costs associated with the OPEX Injection phase, focused efforts on optimizing this step could yield both environmental and financial benefits. Exploring more efficient injection techniques, better machinery, the use of sustainable fuels such as hydrogen, bio-methane, e-methane, bio-methanol, e-methanol, blue ammonia, e-ammonia, bio-oils, and e-diesel or even alternative methods could be beneficial. Moreover, given the significant cost associated with raw material capture despite its low environmental impact, there might be opportunities for cost savings in this segment. In summary, while the mCDR approach shows promise in terms of carbon sequestration, targeted optimizations, especially in the OPEX Injection and raw material capture phases, could enhance its overall efficacy and cost-effectiveness.

### Early Performance Analysis for an At-Sea Injection Platform

Based on a further preliminary LCA of the embodiment of the herein technology, establishing the use of an injection platform as described in paragraph [V1 199] instead of a modified sea bulk carried vessel as in the previous example conducted in January 2024, here are the foundational assumptions for our analysis. In this system embodiment, the following performance analysis relies on an at-sea injection platform using ocean barges for continuous slurry replenishment at depth of 200 m of water column, as opposed to the previously evaluated model involving port-to-injection site operations operating at depth of 1000 m. This method enhances operational efficiency by minimizing travel and downtime associated with slurry reloading. Here are the foundational assumptions based on a revised LCA conducted in the current year:

| Assumption | | | |
|---|---|---|---|
| Category | Details | Value | Unit |
| Scale | Technology Readiness | Commercial Scale TRL10 | — |
| Biomass | Type | Crops Residual Biomass | — |
| Grinding Step | — | Yes | — |
| Cost | Average cost of Crops | 31.3 | $/ton |
| | Residual Biomass | | |
| Transport | Average Total Distance | 400.5 | km |
| Distance | Biomass to Hub | | |
| Biomass Density | Density of Slurry | 955 | kg/m³ |
| Moisture Content | Included in Transport | 60% | — |
| | Calculations | | |
| Vessel | Type | Injection Platform | — |
| Vessel Capacity | Deadweight Scantling | 9,082 | tonnes |
| Distance | Biomass Transport from Hub to | 40 | km |
| | Injection Platform | | |
| Injection Units | Number per Platform | 12 | — |
| Flow Rate | Injection | 1.75 | m³/min |
| Pump | Power | 84.6 | kWh |
| Specifications | Pressure | 20 | Bar |
| Injection Volume | Per Cycle | 240 | m³ |
| Operation | Total Annual Barge Refills | 475 | Per Year |
| Crew | Number | 39 | Persons |
| | Total Hourly Rate | 761 | $/hr |

### Results Analysis Example 2

| | | Total CO₂ | | |
|---|---|---|---|---|
| | Total CO₂ | Emissions (kg | Percentage | Emission to |
| Production | Emission | per Gross ton of | of Total CO₂ | Removal |
| Steps | (Kg/year) | CO₂ stored) | Emission | Ratio |
| Raw material | 6,775,568 | 3 | 6% | 0.00 |
| (Capture) | | | | |
| OPEX transport | 93,564,239 | 42 | 81% | 0.04 |
| Opex Injection | 15,449,450 | 7 | 13% | 0.01 |
| Total CO2 eq. | 115,789,258 | 51 | 100% | 0.051 |
| Emissions | | | | |
| Gross CO2 | −2,250,790,278 | | | |
| Stored | | | | |
| Net CO2 Balance | −2,135,001,020 | | | |

| | Total Cost | Cost CDR | Percentage of |
|---|---|---|---|
| Production Steps | ($/year) | ($/ton of CO₂) | Total Cost |
| Raw material | 63,322,632 | 29.66 | 49% |
| (Capture) | | | |
| OPEX transport | 34,561,511 | 16.19 | 27% |
| Opex Injection | 21,331,948 | 9.99 | 17% |
| Capex Injection | 393,788 | 0.18 | 0% |
| MRV | 3,588,296 | 1.68 | 3% |
| Third | 1,196,099 | 0.56 | 1% |
| Salary team | 3,973,376 | 1.86 | 3% |
| Total Cost | 128,367,650 | 60 | 100% |

Analysis: From the updated results, it is evident that the cost associated with this modified continuous direct air capture (CDR) approach stands at $60 per tC02, presenting an emission to removal ratio (ERR) of 0.051. This reflects substantial improvements in operational efficiency and environmental impact compared to earlier methods.

Emissions Analysis: In this enhanced system, the bulk of emissions, 81%, now emanates from the OPEX transport phase. This phase surpasses the emissions previously noted in the OPEX Injection phase, which now accounts for only 13% of total emissions. Interestingly, raw material capture still contributes minimally to total emissions, approximately 6%, underscoring the sustained environmental benefits of utilizing residual biomass.

Cost Analysis: In terms of cost distribution, raw material capture has become the largest contributor, accounting for 49% of the total annual cost. This shift is attributed to the higher operational demands and logistics involved in preparing and transporting the biomass. The transport costs, associated with the logistics of barge operations, represent 27% of the costs, while OPEX Injection has reduced to 17% of the total expenditure, due to lower water column depth of injection operation. Additional expenses such as monitoring, reporting, and verification (MRV), third-party services, and personnel salaries collectively contribute 7% to the total cost. Potential for optimizations includes some similar cases as previously established in the previous example.

The foregoing detailed description and accompanying examples are merely illustrative and are not to be construed as limitations on the scope of the invention, which is to be determined solely by the appended claims and their equivalents.

Various changes and modifications to the disclosed embodiments will be apparent to those skilled in the art. Such alterations and modifications, including, but not limited to, chemical structures, substituents, derivatives, intermediates, syntheses, compositions, formulations, or methods of use of the invention, are intended to be within the spirit and scope of the invention.

For all patents, applications, or other reference cited herein, such as non-patent literature and reference sequence information, it should be understood that they are incorporated by reference in their entirety for all purposes as well as for the proposition that is recited. Where any conflict exists between a document incorporated by reference and the present application, this application will control

Headings used in this application are for convenience only and do not affect the interpretation of this application.

Preferred features of each of the aspects provided by the invention are applicable to all of the other aspects of the invention mutatis mutandis and, without limitation, are exemplified by the dependent claims and also encompass combinations and permutations of individual features (e.g., elements, including numerical ranges and exemplary embodiments) of particular embodiments and aspects of the invention, including the working examples. For example, particular experimental parameters exemplified in the working examples can be adapted for use in the claimed invention piecemeal without departing from the invention. For example, for materials that are disclosed, while specific reference of each of the various individual and collective combinations and permutations of these compounds may not be explicitly disclosed, each is specifically contemplated and described herein.

The forgoing aspects of the invention, as recognized by the person having ordinary skill in the art following the teachings of the specification, can be claimed in any combination or permutation to the extent that they are novel and non-obvious over the prior art-thus, to the extent an element is described in one or more references known to the person having ordinary skill in the art, they may be excluded from the claimed invention by, inter alia, a negative proviso or disclaimer of the feature or combination of features.

What is claimed is:

1. A system for the marine sequestration of carbon by injecting a slurry of residual biomass into deep-sea sediments, comprising of:
a. a bulk carrier vessel, equipped with residual biomass storage units, transport seaways, crane systems, injection needle, flexible pipe systems, winch systems, pumping systems, slurry mixer, and energy generation onboard;
b. a flexible pipe system, uniquely designed to transport the slurry from the storage units to the sea floor, adaptable to varying depths and conditions, reducing the energy of the overall injection process; and
c. an adapted ship, with specific modifications for biomass transport, including compartmentalized storage areas, onboard equipment aiding in biomass loading and unloading, and systems to maintain biomass quality during transit.

2. The system of claim 1, wherein has developed an efficient, safe, and minimally capital-intensive technology for marine carbon dioxide removal (mCDR), This technology consists of (1) the gathering of organic biomass, (2) its transportation to and from the port to the injection area, and (3) the formation of slurry and pump injection, With a very low capital expenditure and infrastructure requirement per ton of CO₂ equivalent injected, this system offers a competitive advantage over other efficient systems, such as DAC, Biochar or any industrial-based CDR systems, enabling rapid scalability and potential widespread adoption.

3. The system of claim 1, wherein the flexible pipe system is the principal embodiment of the innovation, including embodiments like bulk biomass transport via conveyor piping with hydrostatic pressure adaptation, and spiral piping for biomass transportation.

4. The system of claim 1, wherein a flexible pipe is utilized, allowing the system to bypass the need for a high pump pressure system that would otherwise have to inject 100 bars of pressure due to the hydrostatic differential at the inlet and outlets of the pumps, This feature is one of the key processes that make this innovation efficient.

5. The system of claim 1, wherein the adapted ship includes unique modifications such as automated systems for seamless loading and unloading of biomass onto ships, nearby storage facilities for short-term storage, and environmental considerations to prevent accidental biomass spillage.

6. The system of claim 1, wherein the energy considerations onboard the vessel are designed to be cost-efficient with an emphasis on renewable energy sources, utilizing state-of-the-art generators and alternative energy sources for near-shore operations.

7. The system of claim 1, wherein the injection operation utilizes a sophisticated sea vessel carbon storage delivery system, allowing the integration of flexible tubing and corer needle, deployment mechanisms, ship positioning, stabilization, and adjustable depth of needle deployment.

8. The system of claim 1, wherein the injection process includes detailed aspects such as flow rate of injection, duration, corer needle penetration and lifting time, all optimized for efficient operation and minimal environmental impact.

9. The system of claim 2, wherein the flexible pipe system includes unique embodiments for increased simultaneous injection capacity, telescopic reels and rail systems, integration with algae bloom recovery, and potential for nearshore operation.

10. The system of claim 3, wherein the adapted ship considers fuel for vessels that prioritize cleaner alternatives such as biofuels, liquid natural gas (LNG), and electric propulsion.

11. The system of claim 1, wherein the operational parameters of marine transportation rely on aspects like vessel speed, shipment frequency, optimized turnaround times at ports, and strict adherence to global maritime regulations.

12. The system of claim 1, wherein the scientific basis for carbon sequestration includes geological storage, deep-sea sediment sequestration, benefits of biomass encapsulation, carbon trapping mechanisms, and advantages over other carbon dioxide removal (CDR) methods.

13. The system of claim 1, wherein the zone of injection details suitable seabed sediments, sediment column volume, detailed sediment characteristics, grain size, and permeability, providing a flexible and comprehensive approach to ocean-based carbon storage.

14. The system of claim 1, wherein the injection location and equipment variation are adaptable to depths between 100-5000 meters and at least 6 km from the shore, with variable size, number, and composition as detailed in specific sections.

15. The system of claim 1, wherein the injection location and equipment variation are adaptable to depths between 5 to 100 meters of water column in the case of near shore injection, at least 6 kilometers from the shore, with variations in size, number, and composition as detailed in specific sections.

16. The system of claim 9, wherein simultaneous injections are carried out vertically or horizontally through the use of specialized injection needle designs and high-efficiency modified manifolds that enable injection pumps to manage multiple injections.

17. The system of claim 1, wherein variations in injection equipment include mobile injection platforms and other similar types of injection vessels as further described in the aforementioned invention.

18. The system of claim 1, step c; claims 5 and 17 can feature an H-shaped platform or other specialized designs for enhanced biomass delivery, which increase annual injection capacity by extending operation times without the need for the platform to return to port for refills, In another embodiment, this H-shaped platform allows for the direct docking of refilling vessels, eliminating ship-to-ship (STS) transfer operations of the biomass, thereby further optimizing carbon storage operation efficiency.

19. The system of claim 3, wherein configurations for shallower water injection do not require the use of conveyor piping with hydrostatic pressure adaptation or spiral piping for biomass transportation, both of which are embodiments designed to enhance injection at deeper water column depths of 200 meters or more.

20. The system of claim 1, wherein the plurality of steps involved in the process for this novel system of marine carbon dioxide removal (mCDR) generally comprises, but is not limited to, the following:
a) Transport of Biomass: The transportation of biomass or any carbon-rich material from its source, such as agricultural hubs or major biomass collection sites, to a designated port, This phase may optionally involve temporary storage, depending on logistical requirements,
b) Transfer to Injection Vessels: The transfer of the carbon-rich organic biomass from the port to the injection vessels, which may include, but is not limited to, the loading and unloading process into transit vessels or directly into the injection vessels, In certain embodiments, this step may be bypassed through the use of fluvial transport, allowing direct transfer from the hub to the injection platform (see detailed description for further embodiments),
c) Biomass Preparation: The preparation of the biomass, wherein it is mixed with seawater, typically onboard the ship, to create a slurry. In embodiments where the biomass is not already in a slurry phase, this process is adapted depending on the type of biomass and specific operational parameters (see detailed description for further elaboration).
d) Slurry Injection: The injection of the prepared slurry into seabed sediments using a variety of injection mechanisms, including, but not limited to, simultaneous vertical and horizontal injection via a sinkcore injector. This phase may incorporate different configurations, such as vibro core or modified gravity core mechanisms, tailored to the prevailing environmental conditions.
e) Permanent Carbon Sequestration: The permanent storage of the injected biomass within marine sediments, leveraging natural geochemical parameters to ensure safe and long-term sequestration of carbon. This step guarantees that the carbon remains securely sequestered, thereby preventing its re-emission into the atmosphere.

21. A method for carbon dioxide removal and long-term sequestration in marine sediments, characterized by a specific sequence of targeted natural processes, comprising:
a. Deep Burial and Initial Oxic Degradation: The method begins with the injection of a slurry composed of ligno-cellulosic biomass and seawater into deep-sea sediments, initiating the first stage of biomass degradation under oxic conditions. This step is critical for breaking down complex macromolecules, such as lignin, using oxygen as the primary electron acceptor.
b. Transition Through the Redox Cascade: As oxygen is depleted, the method ensures that degradation progresses through a sequence of less energetically favorable electron acceptors-nitrate, iron (III) oxides, manganese oxides, and sulfate-culminating in methanogenesis. This controlled progression slows down the degradation process, preserving the biomass for extended periods.
c. Encapsulation and Anoxic Preservation: The method encapsulates the biomass within an anoxic layer of sediment, where limited oxygen exposure time (OET) and sediment compaction mimic natural conditions, such as those observed in the Bengal Fan, to effectively prevent further degradation of the organic carbon.
d. Formation of Stable Carbon Compounds: During the redox cascade, the method promotes the formation of stable carbon compounds, including methane and CO₂ hydrates, which become trapped within the sediment matrix, ensuring their long-term sequestration.
e. Sediment Compaction and Limited Solute Diffusion: The method induces sediment compaction around the injected biomass, significantly limiting the diffusion of solutes and substrates, which further stabilizes the carbon and ensures its preservation for millions of years.
f. In a certain embodiment, the Microbial Interactions and Chemical Additives: The method may incorporate specific microbial communities or chemical additives designed to steer the biochemical reactions towards the formation of highly stable carbon forms, such as biochar or carbonates, thus enhancing the permanence of carbon sequestration.

22. The method of claim 21, wherein the sequence of natural processes is achieved and enhanced through the use of an engineered system, comprising:
a. Injection Vessel and Sinkcore Injector Needle: The system includes a specialized injection vessel equipped with a sinkcore injector needle designed to precisely deliver the biomass slurry into marine sediments at targeted depths, ensuring optimal interaction with the sediment environment.
b. Biomass Slurry Composition and Control: The system features a carefully formulated slurry of ligno-cellulosic biomass, optimized in particle size and chemical composition to induce the desired natural reactions within the sediment. The slurry composition is engineered to match the environmental conditions, promoting the transition through the redox cascade.
c. Controlled Injection Parameters: The system employs advanced controls over injection parameters, including flow rates, pressure, and timing, which are critical for ensuring that the injected biomass interacts with the sediment in a way that facilitates the sequence of natural processes described in claim 21.
d. Induced Sediment Compaction: The system is designed to induce sediment compaction around the injected biomass, using the pressure and injection techniques described, to further enhance carbon stability and limit solute diffusion, replicating the natural compaction observed in environments like the Bengal Fan.
e. In further embodiment, the Integration of Microbial and Chemical Additives: The system may integrate specific microbial communities or chemical additives into the slurry to direct the biochemical processes within the sediment, promoting the formation of stable carbon compounds and ensuring the long-term sequestration of carbon.

23. A system of claim 20, wherein a SinkCore Injection Needle: A SinkCore Injection Needle system designed for the marine sequestration of carbon, characterized by its ability to inject a slurry of biomass into deep-sea sediments, wherein the needle system employs a modified gravity core mechanism that allows for injection rather than coring, providing a novel method for carbon sequestration.

24. A system of claim 20, wherein a Vibro Needle Core system integrated into a marine carbon sequestration platform, characterized by its capability to use vibratory motions to penetrate deep-sea sediments and inject biomass slurry, wherein the design is optimized for minimal environmental disturbance and maximum injection efficiency.

25. A system of claim 20, wherein an injection platform for marine carbon sequestration, comprising an integrated system of biomass storage, mixing, and injection mechanisms, uniquely configured on a vessel with an A-frame deployment system and flexible piping for simultaneous multi-point injection, providing a novel method for large-scale biomass injection into deep-sea sediments.

26. A system of claim 20, wherein a system for forming a biomass slurry for marine carbon sequestration, characterized by the use of seawater as the primary medium for slurry formation, eliminating the need for additional freshwater resources
